(19) United States
(12) Patent Application Publication
(10) Pub. No.: US 2024/0299254 A1
(43) Pub. Date: Sep. 12, 2024

(54) AUTOMATED SYSTEMS AND METHODS FOR ASSEMBLING DOSES OF A MEDICATION

(71) Applicant: Opio Connect, Inc., Whitefish, MT (US)

(72) Inventors: Michael Pokorny, Whitefish, MT (US); Amber Norbeck, Whitefish, MT (US); Bradley Bauer, Whitefish, MT (US); Thomas Hoffmann, Whitefish, MT (US); Steve Schick, Whitefish, MT (US)

(21) Appl. No.: 18/668,044

(22) Filed: May 17, 2024

Related U.S. Application Data
(63) Continuation-in-part of application No. 18/613,580, filed on Mar. 22, 2024.
Application No. 18/613,580 is a continuation-in-part of application No. PCT/US22/76957, filed on Sep. 23, 2022.
(60) Provisional application No. 63/247,576, filed on Sep. 23, 2021.
(60) Provisional application No. 63/340,844, filed on May 11, 2022.
(60) Provisional application No. 63/353,389, filed on Jun. 17, 2022.

(51) Int. Cl. A61J 7/00; G16H 10/60; G16H 20/13; G16H 80/00

(52) U.S. Cl. CPC A61J 7/0076; G16H 10/60; G16H 20/13; G16H 80/00

(57) ABSTRACT

An automated system for preparing and assembling a collection of doses of a medication, such as liquid medication including but not limited to methadone and buprenorphine, for fast and accurate order fulfillment. The automated system and its components may be configured to occupy a small footprint for integration in a clinic. The automated system may feature a system for dispensing medication into medication bottles, capping and labeling the bottles, and organizing or collating a collection or group of medication doses into an array for fulfillment of the medication order.

### CROSS-REFERENCES TO RELATED APPLICATIONS

This application is a continuation-in-part and claims benefit of U.S. application Ser. No. 18/613,580 filed Mar. 22, 2024, which is a continuation-in-part and claims benefit of International Application No. PCT/US22/76957 filed Sep. 23, 2022, which claims benefit of U.S. Provisional Application No. 63/247,576 filed Sep. 23, 2021, U.S. Provisional Application No. 63/340,844 filed May 11, 2022, and U.S. Provisional Application No. 63/353,389 filed Jun. 17, 2022, the specifications of which are incorporated herein in their entirety by reference.

### FIELD OF THE INVENTION

The present invention relates to systems and methods for assembling doses of medication, more particularly to an automated system capable of preparing medication doses and collating a collection of doses. The medications include but are not limited to controlled substances such as methadone, buprenorphine/naloxone, and buprenorphine.

### BACKGROUND OF THE INVENTION

The use of opioids has affected the lives of millions of people. There are several existing medications and treatments recommended by the World Health Organization and other healthcare professionals, such as buprenorphine and methadone. Methadone can only be dispensed at a federally registered Opioid Treatment Program (OTP) whereas buprenorphine/naloxone and buprenorphine can be dispensed at a pharmacy or an OTP. The present invention features automated systems and methods for preparing and assembling a collection of doses of medication such as but not limited to doses of methadone and/or buprenorphine. The automated systems can receive orders locally or remotely. The automated system can be used independently in clinics, pharmacies, FQHCs, etc., to support dose assembly and receive local or remote orders.

The present invention also features compliant safe systems. Methadone is a Schedule II controlled substance that must be stored in a controlled substance safe per Title 21 CFR § 1301.72. Typically, OTPs use an Underwriters Laboratory (UL) 687 approved TL-15 or TL 30 safe to store their medication. This is universally accepted to satisfy Title 21 CFR § 1301.72 requirements; however, the Controlled Substances Act (CSA) Title 21 CFR § 1301.72 does not explicitly require UL 687 compliance. This allows for the development of a remote automated system with a compliant safe capable of storing methadone, buprenorphine, buprenorphine/naloxone, and other high-level controlled substances and dispensing said controlled substances without the need to open the safe. The present invention also features remote automated systems with a compliant safe capable of storing and dispensing (and assembling orders received locally or remotely) of medication, e.g., controlled substances such as methadone, buprenorphine, etc., without the need to open the safe.

The present invention also features remote treatment booths (booth systems) for providing a remote interface between healthcare professionals and patients, which may be used for securely delivering opioid treatment doses. In some instances, medications are combined with behavioral counseling in order to monitor the patient's progress, a treatment style that has been proven to decrease opioid use and opioid-related overdose deaths, as well as increase social functioning and retention in treatment. Treating opioid abuse in this way requires frequent communication between a healthcare professional and a patient as well as close monitoring of treatment dosage and delivery. However, it can be difficult for patients to travel to a medical center at the frequency required for effectively controlled and monitored treatment. The present invention describes a booth system for allowing opioid recovery patients to receive communication and treatment from a healthcare professional efficiently without needing to travel to the home OTP for every encounter. The booth system can receive orders locally (e.g., for in clinic) or remotely (e.g., when the booth system is remote). For example, the booth system can receive orders remotely from a healthcare provider/OTP (or other appropriate remote provider) when the booth system is remote.

The automated systems described herein can be used independently from the Booth system as a standalone system to support dose assembly in Opioid Treatment Programs, pharmacies, FQHCs, etc. and can receive orders locally or remotely.

### BRIEF SUMMARY OF THE INVENTION

The present invention features automated systems and methods for preparing and assembling a collection of doses of medication. The systems and methods herein feature a software system configured to work with electronic medical record systems. The systems and methods allow healthcare professionals to send a medication order from the electronic medical record for a collection of doses of a particular medication for a patient and have the doses prepared and assembled or collated quickly and accurately.

While the examples described herein feature liquid medication, the system is not limited to liquid medication and may be modified to dispense and process other types of medications such as solid or sublingual medications. Further, the present invention is not limited to controlled substances such as methadone and buprenorphine.

The automated system may be constructed with its components or a subset of its components organized inside a frame or housing. The automated system and its components may be designed to occupy a small footprint for integration in a clinic such as an OTP clinic or other healthcare setting.

The components of the automated system may include but are not limited to one or a combination of: a system for processing medication bottles (e.g., bottle feeding system), a system for processing caps (e.g., cap feeding system), a system for labeling the bottles (e.g., labeling system), a dial table for holding bottles during dispensation of medication and capping of the bottles, a control center (for operating and controlling one or a plurality of the system components), a dispensing system for aliquoting units of medication through a dispenser (optionally featuring a pump or a means of connecting a medication pump), a system for collating a plurality of doses of the medication (e.g., assembling the medication doses), a system for providing access to the assembled medication doses, robotics for moving caps and/or bottles throughout the system, etc.

The automated system can operatively connect to software systems for receiving orders such as but not limited to a cloud-based system or a local software system. For example, the automated system may be operatively connected to a main software system that receives order details from a healthcare professional's workstation, thereby integrating with electronic medical record systems accessed by the healthcare professionals. The main software system may be a cloud-based software system, however the present invention is not limited to a cloud-based main software system. For example, the main software system may be run on a local server. Receipt of the order details by the automated system from the main software system provides the instructions to the control center of the automated system to allow for preparation of the doses and assembly of the doses.

In certain embodiments, the automated system is constructed without a pump but is adapted to accept and integrate a pump.

As previously discussed, the automated systems and methods herein are for automated assembling or collating of a plurality of doses of a medication (for a medication order) into a group or array, thereby fulfilling a complex medication order. The systems described herein are configured to fulfill a queue of medication orders in series, however the present invention is not limited to this configuration. The system operates in real time, e.g., a healthcare professional orders the medication order and the system is activated to fulfill the order. The order becomes available to a healthcare professional upon completion. The methods and systems herein are different from organization systems such as Pyxis or access systems that allow access to medication at particular time intervals and prevent access between the allowable time intervals. Pyxis does not perform dose assembly, but rather houses a large array of medications that healthcare professionals can access at determined intervals based on medication order.

As used herein, the term “collating” may refer to the assembly or organization of a plurality of bottles into a particular group (array), the group (array) being the appropriate number of doses needed to complete a medication order for a patient.

In certain embodiments, the medication featured in the automated system is a liquid medication. In certain embodiments, the medication featured in the automated system is a solid medication. In certain embodiments, the medication featured in the automated system is a controlled substance.

The present invention features an automated system for assembling an array of doses of a medication, wherein the array of doses comprises one or a plurality of bottles each filled with a predetermined dose of the medication. In some embodiments, the system comprises a control center for receiving an order for an array of doses of the medication from an electronic medical record (EMR) system; a preparation system for filling the one or the plurality of bottles with the predetermined amount of medication and capping and labeling said one or plurality of bottles; and an assembly system for collating the one or the plurality of bottles with the predetermined amount of medication into the array. The system may further comprise the main software system operatively connected to the control center, the main software system is configured to receive the order for the array of doses of the medication from the EMR system and transmit a signal to the control center to provide instructions for preparing and assembling the array of doses of the medication. The main software system is configured to queue a plurality of orders for arrays of doses of medication and organize instructions to the control center for preparation and assembly of said plurality of orders of arrays of doses of medication. In some embodiments, the main software system removes patient personal information from the order sent by the EMR system and assigns a unique ID to each order prior to transmitting the signal to the control center of the automated system. In some embodiments, the preparation system and assembly system are operatively connected to the control center so as to receive instructions therefrom. In some embodiments, the preparation system comprises a dispensing system configured to dispense a predetermined dose of medication into bottles, a capping system configured to apply caps to bottles, and a labeling system configured to apply a label to each bottle. In some embodiments, the dispensing system, capping system, and labeling system are operatively connected to the control center so as to receive instructions therefrom. In some embodiments, the control center is operatively connected to the EMR system via a main software system configured to accept an order from the EMR system and provide instructions to the control center of the automated system to assemble the order. In some embodiments, the preparation system and assembly system are housed in a frame enclosure. In some embodiments, the system is configured to allow a medication pump to be integrated.

The present invention also features an automated system for assembling an array of doses of a medication (the array of doses comprising one or a plurality of bottles each filled with a predetermined dose of the medication). In some embodiments, the system comprises a means for receiving an order for an array of doses of the medication; a means of filling one or a plurality of bottles with the predetermined amount of medication; and a means for collating the one or the plurality of bottles with the predetermined amount of medication into an array. In some embodiments, the means for receiving an order for an array of doses of the medication, the means of filling one or a plurality of bottles with the predetermined amount of medication, and the means for collating the one or the plurality of bottles with the predetermined amount of medication into an array are housed in a frame enclosure. In some embodiments, the system is configured to allow a medication pump to be integrated.

The present invention also features an automated system for assembling a collection of doses of a medication wherein the system comprises a control center for receiving an order for an array of doses of the medication from an electronic medical record (EMR) system; a dispensing system configured to dispense a predetermined dose of medication into bottles; a capping system configured to apply caps to bottles; a labeling system configured to apply a label to each bottle; and an order buffer system for collating the one or the plurality of bottles with the predetermined amount of medication into the array. In some embodiments, the dispensing system, capping system, labeling system, and order buffer system are operatively connected to the control center so as to receive instructions therefrom to assemble the collection of doses of the medication. In some embodiments, the control center is operatively connected to the EMR system via a main software system configured to accept an order from the EMR system and provide instructions to the control center of the automated system to assemble the order. In some embodiments, the dispensing system, capping system, labeling system, and order buffer system are housed in a frame enclosure. In some embodiments, the system assembles the collection of doses of the medication in real time. In some embodiments, the system is a medication pump accessory system. In some embodiments, the system is adapted to integrate a medication pump. In some embodiments, the system comprises a dial table system comprising at least four slots therein for temporarily holding a medication bottle, wherein each of the at least four slots occupies either a loading position, a dispensing position, a capping position, or an unloading position, the dial table system rotates to move slots from one position to another. In some embodiments, the labeling system is configured to obtain a bottle in the unloading position of the dial table. In some embodiments, the capping system is configured to obtain a cap from a cap pickup location and securely attach the cap to a medication bottle in the capping position of the dial table system. In some embodiments, the dispensing system is configured to aliquot a unit of medication from a reservoir through a dispenser into a medication bottle in the slot occupying the dispensing position of the dial table system. In some embodiments, the system comprises a tray drawer system for accepting an array. In some embodiments, the tray drawer provides a user access to the order array. In some embodiments, the system comprises a bottle feeding system for obtaining a medication bottle from a bottle storage hopper. In some embodiments, the system comprises a bottle feeding system for obtaining a medication bottle from a bottle storage hopper and directing the medication bottle to a slot occupying the loading position of the dial table system. In some embodiments, the system comprises a cap feeding system for obtaining a cap from a cap storage hopper. In some embodiments, the system comprises a cap feeding system for obtaining a cap from a cap storage hopper and directing the cap to a cap pickup location.

The present invention also features an automated system for assembling a collection of doses of a medication wherein the system comprises a control center for receiving an order for an array of doses of the medication from an electronic medical record (EMR) system; a dispensing system configured to dispense a predetermined dose of medication into bottles; a capping system configured to apply caps to bottles; a labeling system configured to apply a label to each bottle; and an order buffer system for collating the one or the plurality of bottles with the predetermined amount of medication into the array. The system may further comprising one or a combination of: (i) a dial table system comprising at least four slots therein for temporarily holding a medication bottle, wherein each of the at least four slots occupies either a loading position, a dispensing position, a capping position, or an unloading position, the dial table system rotates to move slots from one position to another; (ii) a bottle feeding system for obtaining a medication bottle from a bottle storage hopper and directing the medication bottle to the slot occupying the loading position of the dial table system; (iii) a dispensing system for aliquoting a unit of medication from a reservoir through a dispenser into a medication bottle in the slot occupying the dispensing position of the dial table system, the dispensing system comprises a means of connecting a medication pump; (iv) a cap feeding system for obtaining a cap from a cap storage hopper and directing the cap to a cap pickup location; (v) a capping system for obtaining a cap from the cap pickup location of the cap feeding system and securely attaching the cap to a medication bottle in the capping position of the dial table system; (vi) a labeling system for obtaining a bottle from the unloading position of the dial table system and labeling said bottle; (vii) an order buffer system for accepting bottles from the labeling system and organizing one or a plurality of bottles as an order array; and/or (viii) a tray drawer system for accepting an order array from the order buffer system and providing a user access to the order array. In some embodiments, the automated system is operatively connected to a main software system, which is operatively connected to an electronic medical record (EMR) system. In some embodiments, the main software system is a cloud based software system. In some embodiments, the main software system is operatively connected to the EMR system via a clinic cloud software system. In some embodiments, the main software system is configured to receive a medication order from the EMR system and provide instructions to the automated system for filling the medication order. In some embodiments, the EMR system is accessible via a user interface on a healthcare professional's workstation. In some embodiments, the user interface is operatively connected to a clinic's cloud based software. In some embodiments, the main software system is operatively connected to an application for a healthcare professional's workstation.

The present invention also features a medication pump accessory system for automated assembling of an array of doses of a medication. In some embodiments, the system comprises a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; and a capping system for securely attaching a cap to a medication bottle in the capping position of the dial table system. In some embodiments, the control center is operatively connected to the EMR system via a main software system configured to accept an order from the EMR system and provide instructions to the control center of the automated system to assemble the order.

The present invention also features a medication pump accessory system for automated assembling of a medication order wherein the system comprises a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; a capping system for securely attaching the cap to a medication bottle in the capping position of the dial table system; and a labeling system for obtaining a bottle from the unloading position of the dial table system and labeling said bottle. In some embodiments, the control center is operatively connected to the EMR system via a main software system configured to accept an order from the EMR system and provide instructions to the control center of the automated system to assemble the order.

The present invention also features a medication pump accessory system for automated assembling of a medication order wherein the system comprises a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; a capping system for obtaining a cap from the cap pickup location of the cap feeding system and securely attaching the cap to a medication bottle in the capping position of the dial table system; a labeling system for obtaining a bottle from the unloading position of the dial table system and labeling said bottle; an order buffer system for accepting bottles from the labeling system and organizing one or a plurality of bottles as an order array; and a tray drawer system for accepting an order array from the order buffer system and providing a user access to the order array. In some embodiments, the control center is operatively connected to the EMR system via a main software system configured to accept an order from the EMR system and provide instructions to the control center of the automated system to assemble the order.

The present invention also features a method of assembling an array of doses of a medication. In some embodiments, the method comprises providing an order for an array of doses of a medication from an electronic medical record (EMR) system to a main software system operatively connected to a control center of an automated system for assembling arrays of doses of medication as described herein. As an example, in some embodiments, the automated system comprises a control center for receiving an order for an array of doses of the medication from the electronic medical record (EMR) system via the main software system; a preparation system for filling the one or the plurality of bottles with the predetermined amount of medication and capping and labeling said one or plurality of bottles; and an assembly system for collating the one or the plurality of bottles with the predetermined amount of medication into the array. With respect to the method, providing the order from the EMR system triggers the main software system to send instructions to the control center of the automated system that allow for the automated system to prepare and assemble the array of doses of the medication. In some embodiments, the main software system is a cloud based software system. In some embodiments, the main software system is run on a local server. In some embodiments, the main software system is operatively connected to the EMR system via a clinic cloud software system. In some embodiments, the EMR system is accessible via a user interface on a healthcare professional's workstation. In some embodiments, the user interface is operatively connected to a clinic's cloud based software. In some embodiments, the main software system is operatively connected to an application on a healthcare professional's workstation.

The present invention also features a cap feeding system. In some embodiments, the system comprises a belt feeder comprising a vertically angled motor-driven belt with cleats spaced a distance apart, the cleats are adapted to support at least one cap; wherein a lower end of the belt feeder engages caps in a cap storage hopper such that as the belt rotates the cleats rise through caps in contact in the storage hopper to allow one or more caps to rest on the cleats as they rise, and caps move toward a top end of the belt feeder. The system may further include the cap storage hopper. In some embodiments, the system further comprises a delivery guide for directing caps at the top end of the belt feeder to a cap pickup location.

The present invention also features a cap feeding system comprising a belt feeder comprising a belt that rotates around an elongated plate or elongated axis formed by a drive gear operatively connected to a drive motor and a belt wheel; and a plurality of cleats disposed on the belt spaced a distance d1 apart; wherein a lower end of the belt feeder is configured to engage caps housed in a cap storage hopper such that as the belt rotates the cleats rise and capture one or more caps thereon as the cleats rise towards a top end of the belt feeder, wherein the belt feeder is oriented at an angle a1 with respect to horizontal; and a delivery guide that delivers caps from at or near the top end of the belt feeder to a cap pickup location. In some embodiments, the system further comprises a guide plate disposed at or near the top end of the belt feeder, the guide plate moves or causes movement of the caps off of the belt feeder. In some embodiments, the speed of the drive motor can be adjusted to accommodate a particular rate of cap delivery to the delivery guide. In some embodiments, angle a1 is such that only caps in a correct orientation rise from the lower end of the belt feeder to the top end of the belt feeder and caps in an incorrect orientation fall off of the belt as the cleats rise. In some embodiments, angle a1 is 80 degrees. In some embodiments, angle a1 is from 50 to 85 degrees. In some embodiments, distance d1 allows caps to be loaded only along the cleats. In some embodiments, distance d1 is 50 mm. In some embodiments, distance d1 is from 40 to 60 mm. In some embodiments, the guide plate comprises a surface having an angle a2. In some embodiments, angle a2 is 45 degrees. In some embodiments, angle a2 is from 40 to 50 degrees. In some embodiments, the delivery guide comprises a chute through which caps travel from the belt feeder to the cap pickup location, the chute is configured to allow gravity to cause the cap to rotate upon exiting the belt feeder and become oriented with its open end facing downward. In some embodiments, the system further comprises one or more sensors for detecting one or a combination of events: presence or absence of a cap at the cap pickup location; proper or improper orientation of a cap in the delivery guide or at the cap pickup location; a backup of caps in the delivery guide. The system may include the cap storage hopper. In some embodiments, the cap storage hopper comprises a fill opening for providing access to its inner cavity to allow refilling of caps. In some embodiments, the system further comprises one or more sensors for detecting if the number of caps in the cap storage hopper is below a predetermined threshold. In some embodiments, the system further comprises a cap reject return component for capturing caps that fall off of the belt feeder to allow said caps to return to the cap storage hopper.

The present invention also features a bottle feeding system. In some embodiments, the system comprises a belt feeder comprising a vertically angled motor-driven belt with cleats spaced a distance apart, the cleats are adapted to support at least one bottle; wherein a lower end of the belt feeder engages bottles in a bottle storage hopper such that as the belt rotates the cleats rise through bottles in contact in the bottle storage hopper to allow one or more bottles to rest on the cleats as they rise, and bottles move toward a top end of the belt feeder.

The present invention also features bottle feeding system comprising a belt feeder comprising a belt that rotates around an elongated plate or elongated axis formed by a drive gear operatively connected to a drive motor and a belt wheel; and a plurality of cleats disposed on the belt spaced a distance d2 apart; wherein a lower end of the belt feeder is configured to engage bottles housed in a bottle storage hopper such that as the belt rotates the cleats rise and capture one or more bottles thereon as the cleats rise towards a top end of the belt feeder, wherein the belt feeder is oriented at an angle a1 with respect to horizontal; and a bottle delivery guide that delivers bottles from the belt feeder to a bottle pickup location. In some embodiments, the system is configured to deliver one bottle per cleat to the bottle pickup location. The system may further comprise a guide plate disposed at or near the top end of the belt feeder, the guide plate moves or causes movement of the bottles to a side edge of the belt feeder. In some embodiments, the bottles exit the belt feeder via an exit point. In some embodiments, the bottles falling off the belt feeder return to a bottle storage hopper via a return chute. In some embodiments, one bottle per cleat can pass the guide plate and reach the exit point. In some embodiments, the system further comprises a bottle delivery guide through which bottles leaving the belt feeder travel to a bottle pickup location. In some embodiments, the system further comprises a pusher wheel system for pushing bottles off of the belt feeder at the exit point, the bottles travel through a bottle delivery guide to a bottle pickup location. In some embodiments, the pusher wheel system comprises a rotating pusher wheel operatively connected to a pusher wheel motor. In some embodiments, the pusher wheel system comprises a flexible drive spring which helps move the bottle into the delivery guide as the bottle passes the rotating pusher wheel. In some embodiments, the speed of the drive motor and pusher wheel system can be adjusted to accommodate a particular rate of bottle delivery. In some embodiments, the belt is oriented at an angle b1 with respect to horizontal. In some embodiments, angle b1 is 80 degrees. In some embodiments, angle b1 is from 50 to 85 degrees. In some embodiments, distance d2 is 1.875 inches. In some embodiments, distance d2 is from 1 to 3 inches. In some embodiments, the guide plate comprises a surface having an angle b2. In some embodiments, angle a2 is 30 degrees. In some embodiments, angle a2 is from 10 to 60 degrees. In some embodiments, the bottle delivery guide comprises a gravity chute and a selector disposed at an end of the gravity chute, wherein gravity causes the bottles to travel through the gravity chute to the selector. In some embodiments, the chute can hold a certain number of bottles (e.g., 5, 6, 7, 8-10, 6-12, etc.) as a buffer. In some embodiments, the delivery guide comprises a first bottle sensor in the chute for detecting a backup of bottles. In some embodiments, the selector comprises a rotator wheel operatively connected to a rotator wheel drive motor, the rotator wheel rotates bottles clockwise or counterclockwise to orient a bottle in a correct orientation and subsequently delivers bottles through a second chute. In some embodiments, the correct orientation is when the bottle has its opening facing upwardly. In some embodiments, the selector further comprises a first selector sensor for detecting if the bottle is in the correct position in the rotator wheel and a second selector sensor for detecting orientation of the bottle. In some embodiments, the system further comprises one or more sensors for detecting one or a combination of events: presence or absence of a bottle at the bottle pickup location; proper or improper orientation of a bottle in the delivery guide or at the bottle pickup location; a backup of bottles in the delivery guide. In some embodiments, the system includes the bottle storage hopper. In some embodiments, the bottle storage hopper comprises a fill opening for providing access to its inner cavity to allow refilling of bottles. In some embodiments, the system further comprises one or more sensors for detecting if the number of bottles in the bottle storage hopper is below a predetermined threshold.

The present invention also features a dial table system comprising a rotating dial having a plurality of slots therein for accepting bottles, the plurality of slots comprises a loading position, a dispensing position, a capping position, and an unloading position, the dial table is operatively connected to a motor and is rotatable in at least a first direction around an axis to allow movement of bottes from one position to a next position. In some embodiments, the rotating dial comprises a first slot, a second slot, a third slot, and a fourth slot. In some embodiments, the slots can occupy one of: a first position for allowing a bottle to be loaded; a second position for allowing medication to be dispensed into the bottle; a third position for allowing the bottle to be capped; and a fourth position for allowing the bottle to be unloaded. In some embodiments, the rotating dial can rotate in at least a first direction such that (i) a first slot occupies the first position, the second slot occupies the second position, the third slot occupies the third position, and the fourth slot occupies the fourth position; or (ii) a first slot occupies the second position, the second slot occupies the third position, the third slot occupies the fourth position, and the fourth slot occupies the first position; or (iii) a first slot occupies the third position, the second slot occupies the fourth position, the third slot occupies the first position, and the fourth slot occupies the second position; or (iv) a first slot occupies the fourth position, the second slot occupies the first position, the third slot occupies the second position, and the fourth slot occupies the third position. In some embodiments, the dial table is rotatably attached to a support platform. In some embodiments, the dial table is configured to allow a bottle to be inserted into the first position with its opening facing up. In some embodiments, the dial table is configured to allow medication to be dispensed from a dispenser into a bottle in the second position. In some embodiments, the dial table is configured to allow a bottle in the third position to be capped. In some embodiments, the dial table is configured to allow a bottle in the fourth position to be retrieved. In some embodiments, the system further comprises one or more sensors for detecting one or a combination of events: presence or absence of a bottle in a slot in the first position; proper or improper positioning of a bottle in a slot in the first position; presence or absence of a bottle in a slot in the second position; presence or absence of a bottle in a slot in the third position; presence or absence of a bottle in a slot in the fourth position.

The present invention also features a dispensing system for dispensing a liquid medication into a medication bottle. In some embodiments, the system comprises a reservoir for holding liquid medication; a dispenser arm with an exit port through which medication is aliquoted, the dispenser arm is fluidly connected to the reservoir; a first tube having a first end fluidly connected to the reservoir and a second end adapted to operatively connect to a medication pump such that medication can be pumped from the reservoir; and a second tube having a first end fluidly connected to the dispenser arm and a second end adapted to operatively connect to a medication pump such that medication can be dispensed via the dispenser arm. In some embodiments, the system includes the medication pump integrated therein. In some embodiments, the pump is a standard of care medication pump, e.g., Methaspense, Digispense, CSDS, LabTec, etc. In some embodiments, the system is configured to dispense medication into bottles disposed in a dial table system. In some embodiments, the dispenser arm is operatively connected to a motor and can occupy and move between two or more positions. In some embodiments, the dispenser arm can occupy a first position wherein the dispenser arm is positioned to dispense medication into a bottle held in a dial table system; and a second position wherein the dispenser arm is positioned to dispense a fluid into a calibration flask. In some embodiments, the dispenser arm can occupy a third position wherein the dispenser arm is positioned to dispense a fluid into a waste container or waste funnel. In some embodiments, the system is operatively connected to a control center. In some embodiments, the system comprises a mechanism for operatively connecting a medication pump to the control center, the control center is configured to send instructions to the medication pump for pumping an appropriate volume of medication from the reservoir to and through the dispenser arm. In some embodiments, the system further comprises one or more sensors for detecting proper or improper position of the dispenser arm.

The present invention also features a capping system for receiving a cap, positioning said cap so as to allow attachment to a bottle, and attaching said cap to a bottle. In some embodiments, the capping system obtains the cap from a cap feeding system. In some embodiments, the bottle is immobilized in a dial table when the capping system attaches the cap to the bottle. In some embodiments, the capping system comprises a pick and place system for obtaining a cap and positioning the cap above the bottle and attaching the cap to the bottle; and a bottle clamp system for immobilizing the bottle while the cap is being attached to the bottle by the pick and place system. In some embodiments, the bottle clamp system comprises a bottle clamp cylinder and a bottle clamp motor operatively connected to the bottle clamp cylinder, the bottle clamp motor drives motion of the bottle clamp cylinder between an engaged position and a disengaged position, wherein in the engaged position, the bottle clamp cylinder clamps and immobilizes the bottle, and when the bottle clamp cylinder is in the disengaged position the bottle is not immobilized. In some embodiments, the bottle clamp cylinder comprises a gripping component for providing a grip on the bottle for immobilization. In some embodiments, the pick and place system comprises a rotary gripper for retrieving a cap from a cap pickup location and threading the cap on the bottle, the rotary gripper is operatively connected to at least one motor. In some embodiments, the rotary gripper rotary gripper comprises a torque feedback system for controlled threaded engagement between the cap and bottle, wherein the rotary gripper is configured such that when activated the rotary gripper rotates and lowers the cap at a predetermined rate to allow threads of the cap to engage threads of the bottle until the rotary gripper achieves a threshold torque value, a threshold distance, or both a threshold torque value and a threshold distance. In some embodiments, the rotary gripper is configured to disengage from the cap when the rotary gripper achieves the threshold torque value, the threshold distance, or both the threshold torque value and the threshold distance. In some embodiments, the system further comprises one or more sensors for detecting one or a combination of events: proper or improper positioning of the bottle; proper or improper cap attachment; proper or improper positioning of the clamp cylinder. In some embodiments, the system is configured to divert poorly capped bottles to a quarantine location

The present invention also features a labeling system for labeling filled medication bottles. In some embodiments, the labeling system comprises a robotic arm system for obtaining a bottle in a bottle pickup position and moving the bottle between one or more positions; and a printing system for printing and applying a label on the bottle and monitoring the label thereon. In some embodiments, the robotic arm system is configured to obtain a bottle from the bottle pickup position in a dial table system and move the bottle to a printing position for labeling. In some embodiments, the robotic arm system comprises a gripper that can move between at least an engaged configuration and a disengaged configuration, wherein in the engaged configuration the gripper grips a bottle and in the disengaged configuration the gripper releases the bottle. In some embodiments, the robotic arm system is configured to move the gripper between at least the bottle pickup position and the printing position. In some embodiments, the robotic arm system is configured to move the gripper to the bottle pickup position and the engaged configuration to grip a bottle, then move the gripper to the printing position and the disengaged configuration to release the bottle. In some embodiments, the robotic arm system is configured to maintain the gripper at the printing position and move the gripper to the engaged configuration to grip the bottle, then move the gripper to an order buffer position and the disengaged configuration to release the bottle. In some embodiments, the robotic arm system is configured to maintain the gripper at the printing position and move the gripper to the engaged configuration to grip the bottle, then move the gripper to a quarantine position and the disengaged configuration to release the bottle. In some embodiments, the robotic arm system is configured to move the gripper from the order buffer position to the bottle pickup position. In some embodiments, the robotic arm system is configured to move the gripper from the quarantine position to the bottle pickup position. In some embodiments, the printing system comprises a printer and a label application machine, the printer prints a label and the label application attaches the label to a medication bottle. In some embodiments, the printing system comprises a sensor for detecting presence or absence of a bottle in the printing position. In some embodiments, the label comprises a barcode. In some embodiments, the printing system comprises a scanner for scanning barcodes on labels. In some embodiments, the label features a tab extending from an edge of the label but removable via perforations or other destructive mechanisms.

The present invention also features an order buffer system for assembling a medication order from one or a plurality of filled medication bottles. In some embodiments, the order buffer system is configured to store a medication order until it is retrieved from the system. In some embodiments, the order buffer system is operatively connected to a labeling system which labels medication bottles and a tray drawer system which provides access to a medication order. In some embodiments, a medication order comprises an array of medication bottles. In some embodiments, the order buffer system comprises an array platform for supporting one or a plurality of filled medication bottles placed thereon. In some embodiments, one or more guide rails surround the array platform to prevent bottles from falling off of the array platform. In some embodiments, the system further comprises a mechanism for moving the array to a tray of a tray drawer system. In some embodiments, the mechanism for moving the array comprises a pusher arm system, comprising an elongated arm operatively connected to a motor which drives movement of the elongated arm between at least a first position wherein the elongated arm is retracted behind the array and a second position wherein the elongated arm has moved toward the tray of the tray drawer system sufficiently to move the array completely onto the tray. In some embodiments, the pusher arm system further comprises a drive belt operatively connected to the motor and rotatable around an axis, wherein the motor drives rotation of the drive belt about the axis in a first direction and a second direction opposite the first direction. In some embodiments, the pusher arm system further comprises a support carriage operatively connected to the drive belt and operatively connected to the elongated arm such that rotation of the drive belt via the motor causes the support carriage and elongated arm to move from the first position to the second position and vice versa. In some embodiments, the support carriage rides on carriage guide rails that stabilize the elongated arm as it progresses between at least the first position and the second position. In some embodiments, the system further comprises one or more sensors for detecting proper or improper positioning of the elongated arm. In some embodiments, the order buffer system comprises one or more sensors for providing status of the array. In some embodiments, the order buffer system comprises one or more sensors for detecting a position of the elongated arm. In some embodiments, the order buffer system has the capacity to hold at least 28 medication bottles.

The present invention also features a tray drawer system for accepting a completed medication order in the form of an array of medication bottles. In some embodiments, the tray drawer system is operatively connected to an order buffer system. In some embodiments, the tray drawer system comprises a tray drawer with an inner cavity adapted to accept an order array; a removable tray for supporting an array; and a door disposed in the tray drawer that can move between at least an open position which provides access to the inner cavity of the tray drawer and a closed position which prevents access to the inner cavity of the tray drawer. In some embodiments, the tray drawer comprises a transparent area for providing visual access to the tray drawer. In some embodiments, the tray drawer extends from the frame enclosure. In some embodiments, the door can be temporarily secured in the closed position via a closure component. In some embodiments, the tray drawer system comprises a tray door sensor for determining whether or not the drawer is in the open position or closed position. In some embodiments, the tray drawer system further comprises a door lock or temporarily preventing the door from moving from the closed position to the open position. In some embodiments, the tray drawer system comprises a clearance sensor which scans the inner cavity of the tray drawer to determine if the tray drawer is clear of bottles. In some embodiments, the tray drawer system comprises tray sensors in the tray drawer for determining if the tray is in a proper position or determining if more than one tray is present in the tray drawer.

The present invention also features a control system operatively connected to at least a dispensing system for dispensing a volume or dose of medication into a bottle and operatively connected to a main software system, the main software system is configured to send instructions to the control system for activating the dispensing system to fill a medication order. In some embodiments, the main software system is operatively connected to an electronic medical record (EMR). In some embodiments, the main software system is directly operatively connected to an electronic medical record (EMR). In some embodiments, the main software system is operatively connected to the EMR via an EMR cloud-based software system (application programming interface (API)). In some embodiments, the EMR is operable via a healthcare professional's workstation. In some embodiments, the main software system is operatively connected to a system application operable via a healthcare professional's workstation. In some embodiments, the system application is configured to provide one or a combination of: a patient-associated ID number, an order number, dispensing status, and a dose count. In some embodiments, an order is created with the EMR, the order is communicated through the EMR cloud-based software system and further to the main software system. In some embodiments, the control system is operatively connected to one or a combination of a cap feeding system, a bottle feeding system, a dial table system, a dispensing system, a capping system, a labeling system, an order buffer system, and a tray drawer system. In some embodiments, the control system is operatively connected to a medication pump. In some embodiments, the control system is operatively connected to a label printer. In some embodiments, the control system is operatively connected to a labeling system. In some embodiments, the control system is operatively connected to a local database. In some embodiments, the EMR cloud-based software system is configured to receive an order from the EMR and replace patient information with a unique patient ID. In some embodiments, the EMR is configured to replace patient information with a unique patient ID prior to sending the order to the main software system. In some embodiments, the main software system is configured to receive an order associated with a unique patient ID. In some embodiments, the main software system processes the order and sends instructions to the control system to fill the order. In some embodiments, the control system sends status information to the main software system, whereupon the status information is transmitted to the system application on the workstation. In some embodiments, the control system is configured to send completed status information to the main software system upon order completion. In some embodiments, the main software system is configured to send completed status information to the clinic software system, the clinic software system is configured to send completed status information to the EMR.

The present invention also features a safe system for remote automated dispensation of doses of a medication. In some embodiments, the system comprises a safe housing; a dispense port disposed in the safe housing; one or more dosage containers containing a medication; and one or more dispense mechanisms operatively coupled to the one or more dosage containers and the dispense port. In some embodiments, the one or more dispense mechanisms are operatively connected to the one or more dosage containers via a pump. In some embodiments, the one or more dispense mechanisms are configured to integrate a pump for operative connection to the one or more dosage containers. In some embodiments, the one or more dispense mechanisms are powered and controlled by a plurality of wires directed through the plurality of power and control lead ports. In some embodiments, the system further comprises one or more pump mechanisms operatively coupled to the dosage container or containers and the dispense port. In some embodiments, the system further comprises a plurality of power and control lead ports disposed through the safe housing. In some embodiments, the one or more pump mechanisms are powered and controlled by a plurality of wires directed through the plurality of power and control lead ports. In some embodiments, the pump mechanism is operatively connected to the dosage container by a suction connection. In some embodiments, the suction connection comprises a switch to control suction, wherein the switch is actuated by the computer actuation component. In some embodiments, the pump mechanism is operatively connected to the dosage container by a suction connection. In some embodiments, the suction connection comprises a switch to control suction, wherein the switch is actuated by the computer actuation component. In some embodiments, the system further comprises a computer actuation component operatively coupled to the one or more pump mechanisms, wherein actuating the computer actuation component causes the one or more pump mechanisms to draw a controlled substance dose from the dosage container or containers, and direct the controlled substance dose to the packaging mechanism, then to the dispense port. In some embodiments, the system further comprises a computer actuation component operatively coupled to the one or more dispense mechanisms, wherein actuating the computer actuation component causes the one or more dispense mechanisms to collect a controlled substance dose from the dosage container or containers, and direct the controlled substance dose to the packaging mechanism, then to the dispense port.

The present invention also features a method for automated dispensation of doses of a medication. In some embodiments, the method comprises providing instructions to a safe system as described herein, wherein the safe system is configured to prepare one or more doses of the medication. In some embodiments, the medication is a controlled substance. In some embodiments, the controlled substance comprises methadone, buprenorphine, extended-release naltrexone, or a combination thereof in liquid, tablet, or sublingual form.

The present invention also features an opioid treatment booth system for providing an interface between a user and a healthcare professional. In some embodiments, the booth system comprises a user interface chamber with an interior space configured for a user's presence therein; an interface system; a treatment delivery component configured to obtain and deliver one or more treatment doses from a storage container; and a biometric input processing component capable of accepting a biometric input from the user as a means of authorization, the biometric input processing component is operatively connected to the interface system, wherein when the biometric input processing component authorizes the biometric input of the user, a signal is sent to activate the treatment delivery component to deliver the one or more treatment doses. In some embodiments, the booth further comprises an input component capable of allowing the user to communicate with the healthcare professional and a display component capable of allowing the healthcare professional to communicate with the user. In some embodiments, the booth further comprises a treatment exchange component. In some embodiments, the treatment exchange component is capable of accepting a treatment dose from a treatment containment chamber. In some embodiments, the biometric input of the user is registered to the opioid treatment booth. In some embodiments, the user interface chamber further comprises a disposal component for the user to dispose of a portion of the treatment dose after taking it while in communication with the healthcare professional. In some embodiments, the biometric input processing component further accepts a user ID number in addition to the biometric input as the means of authorization. In some embodiments, the biometric input is a fingerprint, a facial scan, a retinal scan, a palm-vein scan, or a combination thereof. In some embodiments, the input component comprises a touch screen, a keyboard, a microphone, a standard camera, a thermal image, or a combination thereof. In some embodiments, the display component comprises a screen, a speaker, or a combination thereof. In some embodiments, the treatment dose comprises methadone, buprenorphine, buprenorphine/naloxone, or a combination thereof. In some embodiments, the healthcare professional, while connecting to the opioid treatment booth, is able to view the user's ID number, location, wait time, and notifications. In some embodiments, the healthcare professional is able to register a flag to the user if the user attempts to divert treatment. In some embodiments, the healthcare professional is capable of adjusting the user's treatment dose as necessary. In some embodiments, the treatment delivery component delivers the treatment dose by dispensing, coupled to a pump, the treatment dose into a container in a fluid path to be transferred to the user through the treatment exchange component. In some embodiments, the container comprises a bottle, and wherein the treatment delivery component comprises a bottle feeder and a cap feeder to allow the treatment dose to be dispensed into the bottle and for the bottle to be sealed with a cap. In some embodiments, the healthcare professional is able to view a total number of treatment doses contained in the treatment storage container. In some embodiments, the booth further comprises a robotic arm disposed in the treatment containment chamber connected to a computing component comprising a processor capable of executing computer-readable instructions and a memory component comprising computer-readable instructions for: moving the treatment dose from the treatment delivery component to the treatment exchange component; removing an unused treatment dose from the treatment delivery component placing the unused treatment dose into a safe or quarantine zone in the treatment containment chamber; and pouring the unused treatment dose into the disposal component and adjusting the total number of treatment doses contained in the treatment storage container accordingly. In some embodiments, the healthcare professional is able to access the treatment containment chamber for restocking medication. In some embodiments, the booth further comprises a printer, for labeling the container with patient specific information. In some embodiments, the treatment storage container comprises a plurality of individual treatment dose containers, wherein actuating the treatment delivery component causes the pump to extract the treatment dose from an individual treatment dose container, wherein subsequent actuations of the treatment delivery component cause the pump to extract from a different individual treatment dose container. In some embodiments, the treatment storage container comprises a single vat containing the plurality of treatment doses, wherein actuation of the treatment delivery component causes the pump to extract the treatment dose from the single vat.

As used herein, the term “operatively connected” may include any appropriate type of connection including but not limited to a mechanical connection, electrical connection, and/or a wireless connection.

In some embodiments, the system draws less than 20 amps. In some embodiments, the system can be plugged into a standard wall outlet. In some embodiments, the system creates less than 50 dB of noise. In some embodiments, the system has a width that is 32 inches or less. In some embodiments, the system has a height that is 79 inches or less. In some embodiments, the system has a depth that is 43 inches or less. As discussed herein, the present invention is not limited to aforementioned examples of dimensions.

In some embodiments, the bottle storage hopper comprises a door for providing access to its inner cavity. In some embodiments, the frame enclosure comprises a door for accessing the door of the bottle storage hopper. In some embodiments, the cap storage hopper comprises a door for providing access to its inner cavity. In some embodiments, the frame enclosure comprises a door for accessing the door of the cap storage hopper.

The present invention also features a CSA compliant safe, which may be featured as a part of a system for remote automated dose assembly of doses of a medication. The safe may comprise a safe housing with a dispense port and a plurality of power and control lead ports disposed through it. The system within the safe may further comprise a dosage container containing a plurality of controlled substance dosages, a dispensing mechanism, and/or a pump mechanism operatively coupled to the dosage container and the dispense port. The dispensing mechanism and/or pump mechanism may be powered and controlled by a plurality of wires directed through the plurality of power and control ports of the safe.

The system may further comprise a cooling and heating mechanism to maintain a specific ambient temperature within the safe or a specific temperature of the medication stored within the safe. The controlled substance dosages may comprise but are not limited to methadone, buprenorphine, buprenorphine/naloxone or a combination thereof. The dosage forms may comprise but are not limited to liquid, tablet, and sublingual strip form.

The present invention also features a system that allows healthcare professionals to remotely interface with patients, e.g., for securely delivering opioid treatment doses. For example, the present invention features an opioid treatment booth system. In some embodiments, the opioid treatment booth may comprise a user interface chamber for housing a user and allowing the user to interface with a healthcare professional. The user interface chamber may comprise a biometric input component capable of accepting a biometric input from the user, e.g., patient. The user may be registered to a specific opioid treatment booth or authorized to use any booth within a network by their biometric input. Authorizing the biometric input component may actuate an interface system and a treatment delivery component. In some embodiments, the biometric input component may further accept a user ID number in addition to the biometric input as the means of authorization. The user interface chamber may further comprise an input component capable of allowing the user to communicate with the healthcare professional, and a display component capable of allowing the healthcare professional to communicate with the user. The interface chamber may have a series of cameras to allow the healthcare professional to visually observe the patient. The interface system may require the user to answer questions displayed through the display component through the input component. The user interface chamber may further comprise the treatment exchange component capable of accepting a treatment dose from a treatment containment chamber. In some embodiments, the treatment exchange component is locked into a wall of the user interface chamber until authorization has been received from the biometric input component. The opioid treatment booth may further comprise the treatment containment chamber. The treatment containment chamber may comprise one or more treatment storage containers which may comprise a plurality of treatment doses in the oral liquid or oral solid dosage form. The treatment containment chamber may further comprise a treatment delivery component operatively coupled to the treatment exchange component capable of delivering one or more treatment doses to the treatment exchange component. The one or more treatment doses may be labeled as either an observed dose to be taken by the user in the user interface chamber or a take-home dose that does not need to be observed as it is taken.

One of the unique and inventive technical features of the present invention is the dispensation of a controlled substance and incorporating a safe that meets the specifications in the Controlled Substance Act (CSA). Without wishing to limit the present invention to any theory or mechanism, it is believed that the present invention advantageously provides for remote delivery of a controlled substance, in real time through a remote video conference session.

Embodiments of the present invention can be freely combined with each other if they are not mutually exclusive.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A-1B show perspective views of the automated system of the present invention.

FIGS. 2A-2H show the cap feeding system.

FIGS. 3A-3L show the bottle feeding system.

FIGS. 4A-4E show the dial table system.

FIGS. 5A-5D show the dispensing system.

FIGS. 6A-6C show the capping system.

FIGS. 7A-7B show the labeling system.

FIGS. 8A-8E show the order buffer system.

FIGS. 9A-9C show the tray drawer system.

FIGS. 10A-10C show the control system.

FIG. 11A shows an isometric view of a remote automated controlled substance dispensing safe of the present invention.

FIG. 11B shows a view of the safe of the present invention showing the dispense port and the power and control ports.

FIG. 11C shows an interior view of the safe of the present invention showing the dispense port and the power and control ports from the inside.

FIG. 11D shows the interior view of the safe of the present invention showing interior components.

FIG. 12 shows an embodiment of a cable entry assembly that may be used in the ports of the safe of the present invention.

FIG. 13 shows the opioid treatment booth of the present invention.

FIG. 14A shows an external view of the user interface chamber of the opioid treatment booth of the present invention.

FIG. 14B shows an interior view of the user interface chamber of the opioid treatment booth of the present invention.

FIG. 15 shows a view of a disposal component of the opioid treatment booth of the present invention.

FIG. 16 shows a view of a treatment exchange component of the user interface chamber in use.

FIG. 17A shows a view of the opioid treatment booth of the present invention with a panel open to access a treatment containment chamber of the present invention.

FIG. 17B shows an interior view of the treatment containment chamber of the present invention.

FIG. 17C shows an interior view of a treatment storage container of the treatment containment chamber of the present invention.

FIG. 17D shows a pump and cooling component of the treatment delivery component in use.

FIG. 17E shows an alternate view of the pump and cooling component of the treatment delivery component in use.

FIG. 18A shows the treatment delivery component of the present invention bottling and capping a treatment dose.

FIG. 18B shows a robotic arm of the present invention transferring the treatment dose from the treatment delivery component to the treatment exchange component.

FIG. 19A shows a view of an alternate embodiment of the opioid treatment booth of the present invention with a panel open to access a treatment containment chamber of the present invention, wherein the treatment storage container comprises a single vat containing all treatment doses.

FIG. 19B shows a view of the alternate embodiment of the opioid treatment booth of the present invention with the treatment delivery component actuated to pump a treatment dose from the single vat of all treatment doses in the liquid form.

FIG. 19C shows a view of the alternate embodiment of the opioid treatment booth of the present invention with the safe door closed.

FIG. 20 shows a view of the double-step drive plate design of the bottle feeder system of the present invention.

FIG. 21A shows a view of the bottle feeder system of the present invention comprising suitable and unsuitable cleats.

FIG. 21B shows a side view of the cleats of the bottle feeder system of the present invention comprising suitable and unsuitable cleats.

FIG. 22A shows a first view of the clamping system of the present invention.

FIG. 22B shows a second view of the clamping system of the present invention.

FIG. 22C shows a view of the clamping system in line with the dial table system.

FIG. 23A shows a view of the heat sealing system of the present invention.

FIG. 23B shows an alternate view of the heat sealing system of the present invention.

FIG. 23C shows another alternate view of the heat sealing system of the present invention.

FIG. 23D shows a schematic diagram of the heat sealing system of the present invention.

FIG. 24A shows an external view of the heat sealing system control enclosure of the present invention.

FIG. 24B shows an internal view of the heat sealing system control enclosure of the present invention.

FIG. 24C shows an alternate internal view of the heat sealing system control enclosure of the present invention.

FIG. 24D shows another alternate internal view of the heat sealing system control enclosure of the present invention.

### DETAILED DESCRIPTION OF THE INVENTION

The present invention features automated systems (100) and methods for assembling a collection or array of doses of a medication, e.g., a system for receiving an order for a medication wherein the order comprises a plurality of doses of the medication in separate bottles, preparing the collection or array of the doses of medication, and collating/organizing/assembling the collection or array of the doses of medication.

Without wishing to limit the present invention to any theory or mechanism, it is believed that the automated system (100) of the present invention is advantageous because it is adapted for use in clinics, e.g., the automated system (100) is compact enough, quiet enough, and energy efficient enough to be used in clinics. For example, in some embodiments, the automated system draws less than 20 amps so it can be used in a standard 20 amp wall outlet power source. The automated system may be provided with an adequate power cord for easy access to a wall outlet. The automated system may be connectable to software systems wirelessly and/or via connections such as ethernet for local servers or other computer systems. The automated system may be designed to be quiet, e.g., in some embodiments, the automated system creates less than 50 dB of noise. In some embodiments, the automated system is less than 32 inches wide, which allows for movement through a standard door frame. In some embodiments, the automated system is less than 78-79 inches in height. In some embodiments, the automated system is less than 42-43 inches in depth (excluding the tray drawer or other small accessories such as door handles that may affect the actual depth). The automated system may be constructed in a compact manner such that it can be positioned close to the dosing window, e.g, within arms reach or just a short distance for a healthcare professional to access. The configuration of the internal components can allow for the back (e.g, the side opposite the system interface/display) of the frame to be placed against a wall (if necessary) without impeding access for maintenance, etc. The automated system may also feature security measures for limiting or preventing access to the stored medication (e.g., controlled substances including but not limited to methadone, buprenorphine, etc.) while allowing for the dispensing of said medication. The system is easy and intuitive to use, fast, reliable, can be reloaded or refilled easily, and can be implemented with existing electronic medical record (EMR) systems to help manage inventory and send patient dose orders. The system allows for maintaining the same clinical practices with respect to assigning and securing the medication and calibrating and maintaining the medication pump. Note that the automated system is not limited to the aforementioned dimensions or parameters or configurations.

As will be further described herein, the components of the automated system (100) may include but are not limited to one or a combination of: a system for processing medication bottles (e.g., bottle feeding system (300)); a system for processing caps (e.g., cap feeding system (200)); a system for labeling the bottles (e.g., labeling system), e.g., wherein a printer prints labels and the labels are applied to the bottles (the labeling system may be integrated on a pullout drawer for easy access for maintenance and refilling); a dial table system for holding bottles during dispensation of medication and capping of the bottles; a control center (with software, for operating and controlling one or a plurality of the system components and operatively connecting to software that provides orders); a dispensing system for aliquoting units of medication through a dispenser (optionally featuring a pump or a means of connecting a medication pump); robotics for moving caps and/or bottles throughout the system; an interface, e.g., a touchscreen, where users can access information about orders and/or perform calibration and/or maintenance procedures; drawers (e.g., for supporting fluid containers, a printer, a tray, etc.); a tray drawer for presenting orders to the healthcare professional for retrieval; etc.

In certain embodiments, the automated system (100) is constructed without a pump but is adapted to accept and integrate a pump. The ability to integrate a pump into the system allows a clinic to use its current standard of care pumps. The system allows accurate dispensation of the same wide ranges of doses that are currently dispensed in the clinics and allows for the use of the same maintenance and calibration procedures used for the current pumps.

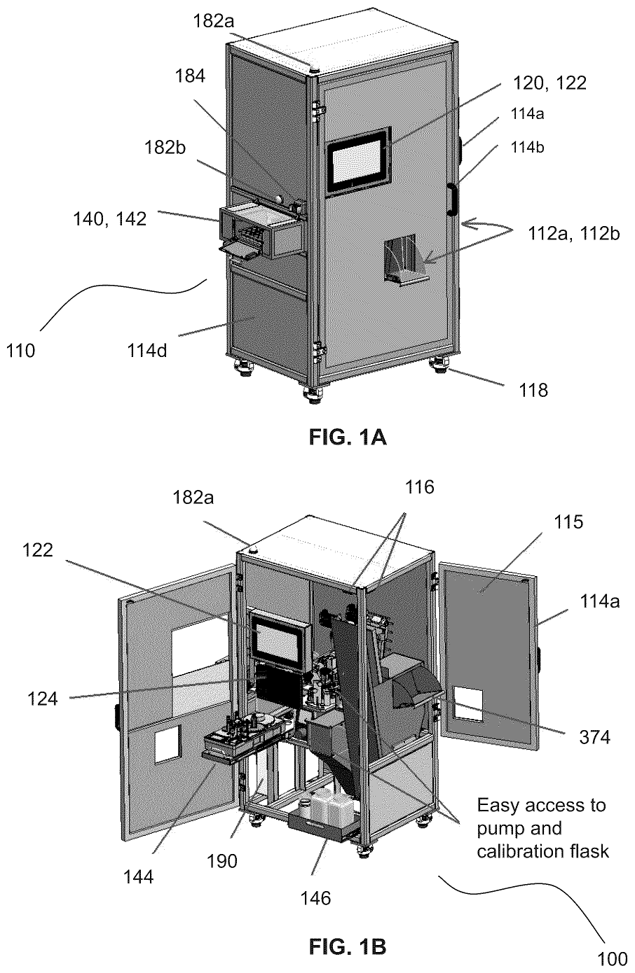

Referring to FIG. 1A and FIG. 1B, as an example, the automated system (100) comprises a frame (110) or housing sized and adapted to be used in a clinical setting. The frame (110) may feature one or more access doors or panels such as a feeder access door (114a) for accessing the cap feeder system and/or the bottle feeder system, a main access door (114a) for accessing one or a plurality of components in the frame (110), a side access door (114c) for accessing one or a plurality of components in the frame (110), and/or access panels (114d) for accessing the control center (190), fluid drawer (146) (e.g., for supporting medication reservoirs (530), other reservoirs (147) for waste, cleaning solution, storage solution, etc.), printer drawer (144), keyboard/mouse tray (124), etc.

In some embodiments, one or more windows or openings are disposed in the frame (110). For example, in the embodiments shown herein, the frame (110) comprises an interface window (120) for providing visual access to a display or interface screen (122) (which may be operatively connected to a keyboard/mouse, e.g., via a hinge), a tray drawer window (140) for providing access to the tray drawer (142). In some embodiments, the frame (110) comprises a first refill door (112a) for providing access to the cap feeder system and/or a second refill door (112b) for providing access to the bottle feeder system. In some embodiments, the frame is equipped with sound-dampening material (115) to reduce sound production by the inner components. In some embodiments, the frame is mounted on wheels (118), e.g., lockable caster wheels. In some embodiments, the frame is equipped with one or more locks (116) for the doors and/or panels.

The interface screen (122) may function to provide information about and/or allow control of calibration and cleaning, information about issues or problems within the system, etc. The interface screen may be used to view video of the internal automation.

In some embodiments, the frame (110) is equipped with one or more status lights, e.g., a machine status light (182a) for relaying information about the status of the automated system, or an order status light (182b) relaying information about an order. In some embodiments, the frame (110) is equipped with an emergency stop button (184) for shutting off the system (100) if needed. The present invention is not limited to the configurations or components shown and described herein.

In some embodiments, the automated system (100) further comprises a cup dispensing system, e.g., for dispensing medication into a cup (in lieu of a capped bottle), which is intended for immediate consumption. In some embodiments, the automated system (100) further comprises a water dispensing system for providing a metered amount of water into a cup dose intended for immediate consumption, or into bottles containing methadone doses as a means of diversion prevention. In some embodiments, the automated system (100) further comprises a dose measuring scale for weighing doses during appropriate times. In some embodiments, the automated system (100) further comprises a control cabinet and power distribution panels.

As will be described herein, the automated system (100) of the present invention is adapted to function with specific software, e.g., a main software system (1050) that connects to the clinic's EMR system and receives order details from the EMR. The main software system (1050) may be a cloud-based system; however, the present invention is not limited to cloud-based software and includes software run on a local server. As an example, orders can be sent from local or remote workstations of a healthcare provider to the main software system (1050), wherein the main software system (1050) buffers orders in a queuing system. When the next order in the queue is ready to be filled, the main software system (1050) sends instructions to the automated system (100) for preparing and assembling the doses. The queuing and other order information may be presented to the healthcare professionals in the form of an application on their workstation. For example, the application may allow the healthcare professional to view orders in the queue, the current order that is being processed, and the complete orders. The application may also function to present warnings or alerts about the system, such as low levels of medication or bottles or caps, etc. The automated system (100) may also be configured to send information back to the main software system (1050) such as when the assembly of an order is completed, and such information may be updated in the applications on the workstations. In some embodiments, there is a local database within the workstation, wherein when the main software system (1050) receives the order completion information from the automated system and sends the required information back to the clinic's EMR.

The present invention also includes systems constructed on a smaller scale, for example systems that can be used on a benchtop or tabletop for small clinics or other healthcare locations that do not have the space capacity or the need for a larger scale system. For example, in some embodiments, the system may have a smaller bottle capacity or cap capacity (e.g., 25 bottles, 50 bottles, 75 bottles, etc.). Creating an automated system for a small scale setting may involve modifications to the system which are within the scope of the invention. As an example, in some embodiments, the cap feeding system and/or bottle feeding system is housed external to the frame. In some embodiments, caps can be manually fed into the system in lieu of an automated cap feeding system. In some embodiments, bottles can be manually fed into the system in lieu of an automated bottle feeding system. In some embodiments, the pump is housed external to the frame. In such an embodiment, the medication may be pumped into the inner compartment of the frame via tubing for dispensation into medication bottles. In some embodiments, the medication reservoir is housed external to the frame. In some embodiments, the pump is housed in an appropriate safe for preventing unwanted access. In some embodiments, the medication reservoir is housed in an appropriate safe for preventing unwanted access.

As previously discussed, the automated system may feature a particular combination of components described herein. For example, in some embodiments, the system comprises (inside the frame) a dispensing system, a capping system, a dial table system or a variation thereof, and a labeling system. In some embodiments, the system may further comprise (inside the frame) an order buffer system. Optionally, other components of the system described herein may be housed external to the frame or modified as needed to maintain the function of the system described herein but occupy a different configuration or scale. Such modifications are within the scope of the invention.

### Cap Feeding System

The cap feeding system is configured to store caps said and deliver caps to the location where the bottles are capped. The caps are delivered in the appropriate orientation and at the appropriate frequency.

To aid in efficiency and ease of use, the cap feeding system may be designed with the following considerations. The cap feeding system may be capable of processing caps when the automated system is running, and the cap storage hopper may be refillable without interfering with dispensation or processing of order. The cap feeding system may have a large capacity to help reduce the frequency it needs to be reloaded. For example, the cap feeding system may be capable of storing up to 1,000 or more caps. In certain embodiments, the cap feeding system features a sensor for detecting when the cap storage hopper is low, and the sensor may trigger an alarm or other alert device to indicate to a user that the cap storage hopper should be refilled.

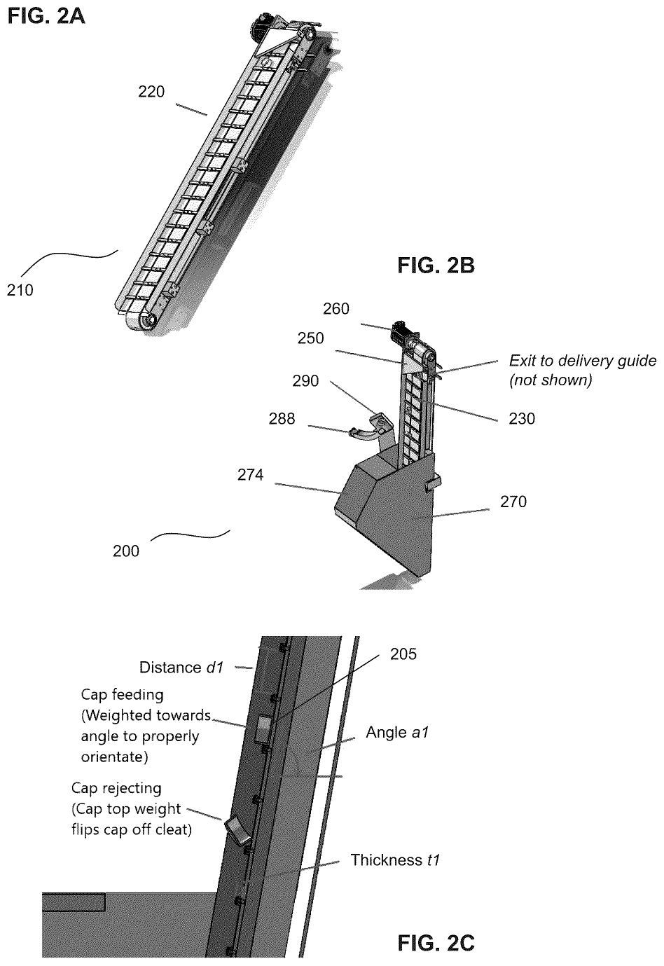

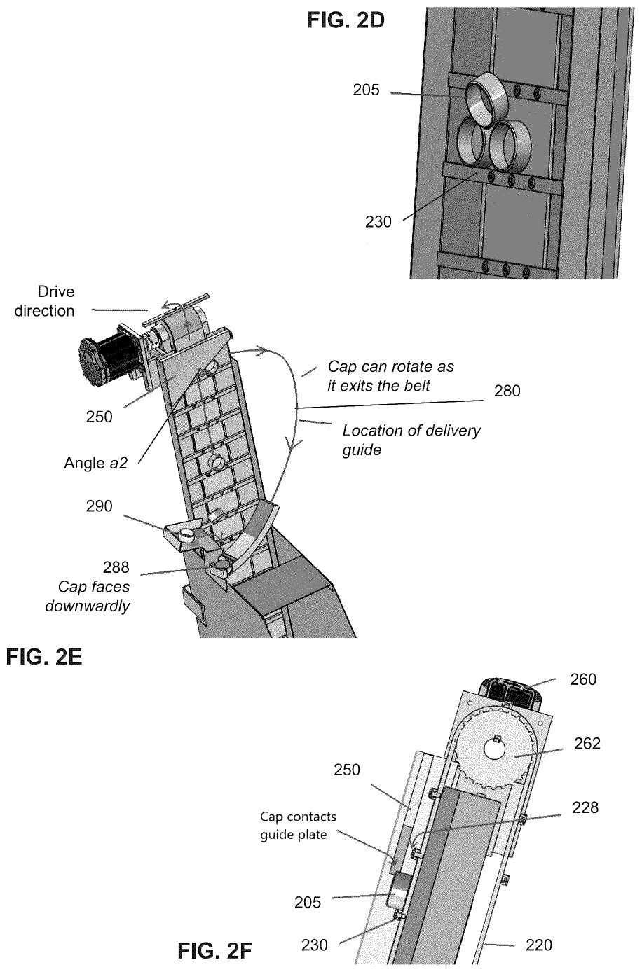

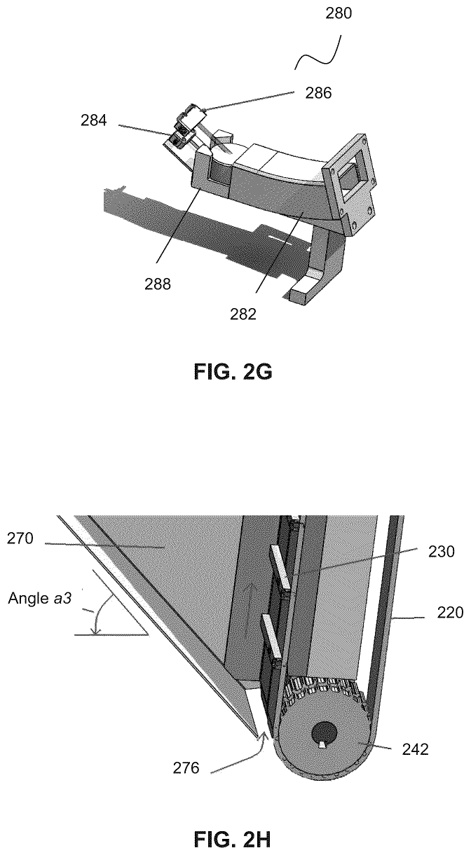

The present invention describes a particular cap feeding system (200) shown in FIG. 2A, FIG. 2B, FIG. 2C, FIG. 2D, FIG. 2E, FIG. 2F, FIG. 2G, and FIG. 2H. The cap feeding system (200) described herein was designed to fit in a compact space within the frame of the automated system (100). It is understood that one of ordinary skill in the art may make modifications to the cap feeding system depending on the cap size, weight distribution, delivery frequency, angle of the belt feeder, etc., and said modifications are within the scope of the present invention.

The cap feeding system (200) in FIG. 2A, FIG. 2B, FIG. 2C, FIG. 2D, FIG. 2E, FIG. 2F, FIG. 2G, and FIG. 2H comprises a cap storage hopper (270) for housing and storing caps, a belt feeder (210) for capturing caps in the cap storage hopper (270), and a delivery guide (280) for delivering caps from the belt feeder (210) to a cap pickup location (288).

The belt feeder (210) comprises a belt (220) that rotates around an elongated plate or elongated axis formed by a drive gear (262) (operatively connected to a drive motor (260)) and a belt wheel (242). A plurality of cleats (230) is disposed on the belt (220) and spaced a distance apart. A guide plate (250) is disposed at or near the top end of the belt feeder (210). The guide plate (250) is a mechanism for moving the caps off of the belt feeder (210) or causing the caps to move off of the belt feeder (210). For example, as the caps progress up the belt (220) on a cleat (230), they contact the guide plate (250), which causes the caps on the cleat to move to the side edge of the belt whereupon the caps exit the belt feeder (220) to a delivery guide (280), which delivers the caps (205) to the cap pickup location (288). In some embodiments, the speed of the drive motor (260) can be adjusted to accommodate a particular rate of cap delivery.

The cleats (230) are spaced a distance d1 apart from each other. The cleats each have a particular width w1, as measured from the first side edge at or near the belt to the second side edge at or near the belt; the width w1 may be similar to the width of the belt (220), and a particular thickness t1, (e.g., depth). The cleats each have a particular height h1. The belt (220) is oriented at an angle a1 with respect to horizontal. The particular angle may be determined based on the size of the caps, the weight of the caps, and the weight distribution of the caps.

As an example, the parameters of the belt feeding system (200) may be designed such that the caps (205) move up the belt feeder (210) with the closed face contacting the belt and the open end facing outwardly. In certain embodiments, the parameters are configured to cause caps that are not oriented properly to fall off back into the storage hopper (270), as shown in FIG. 2C. For example, as shown in FIG. 2C, the angle a1 of the belt feeder (210) may be such that caps only stay on the belt (220) if the opening of the cap faces outwardly because if the closed face of the cap faces outwardly, the weight of the cap causes it to fall off back into the storage hopper (270).

In certain embodiments, the distance d1 between the cleats may be configured to only allow caps to be loaded along the cleat (preventing shingling or stacking of the caps), as shown in FIG. 2D, since shingling or stacking of caps may prevent the caps from exiting the belt feeder properly. In certain embodiments, the distance d1 between the cleats may be configured to only allow 1-2 caps per cleat, however the present invention is not limited to said configuration.

In certain embodiments, angle a1 is 80 degrees. In certain embodiments, angle a1 is from 50 to 85 degrees. In certain embodiments, angle a1 is from 30 to 88 degrees. In certain embodiments, angle a1 is from 50 to 85 degrees. In certain embodiments, angle a1 is from 60 to 85 degrees. In certain embodiments, angle a1 is from 70 to 85 degrees. In certain embodiments, angle a1 is from 70 to 82 degrees. In certain embodiments, angle a1 is from 75 to 85 degrees. In certain embodiments, angle a1 is from 50 to 85 degrees. The present invention is not limited to the aforementioned angle values. The angle may vary depending on the cap size, weight, and weight distribution, among other things.

In certain embodiments, distance d1 is 50 mm. In certain embodiments, distance d1 is from 40 to 60 mm. In certain embodiments, distance d1 is from 45 to 65 mm. In certain embodiments, distance d1 is from 40 to 70 mm. In certain embodiments, distance d1 is from 30 to 80 mm. The present invention is not limited to the aforementioned distance values. The distance may vary depending on the cap size, weight, and weight distribution, among other things.

In certain embodiments, width w1 is 170 mm. In certain embodiments, width w1 is from 100 to 200 mm. In certain embodiments, thickness t1 is 6 mm. In certain embodiments, thickness t1 is from 1 to 20 mm. In certain embodiments, thickness t1 is from 5 to 10 mm. In certain embodiments, thickness t1 is from 5 to 15 mm. The present invention is not limited to the aforementioned width and thickness values, as they may vary depending on the cap size, weight, and weight distribution, among other things, e.g., reduction in noise (e.g., the cleat in the hopper), etc.

The guide plate (250) may be constructed as a surface having an angle a2. In some embodiments, angle a2 is 45 degrees. In some embodiments, angle a2 is from 40 to 50 degrees. In some embodiments, angle a2 is from 30 to 60 degrees. The guide plate (250) is not limited to the particular angle described herein. The guide plate (250) is not limited to the configuration shown and described herein.

The cleats (230) may be constructed from low resistance material to help allow the caps to slide on the cleat towards the delivery guide (280) when contacted by the guide plate (250). A clearance (228) may be present between the cleats (230) and the guide plate (250) such that the caps (but not the cleats (230)) contact the guide plate (250).

The cap storage hopper (270) provides an opening (276) through which the cleats (230) can pass. The opening (276) provides adequate clearance for the cleats (230). The opening (276) is generally smaller than the caps (205) to prevent the caps (205) from inadvertently passing through the opening (276) and/or jamming the system. The cap storage hopper (270) has a fill opening (274) providing access to the inner cavity of the storage hopper (270) to allow for refilling of the cap storage hopper (270). The fill opening (274) may be accessible via a refill door (112) disposed in the frame (110) of the system (100). In some embodiments, the cap storage hopper (270) comprises a sensor for detecting a level or amount of caps (205) therein, In certain embodiments, the sensor is operatively connected to an alert component that is activated (thereby providing a user a signal) when the sensor detects too few caps are in the storage hopper. Sensors configured to sense levels such as caps in a storage unit are well known to one of ordinary skill in the art and include but are not limited to depth sensors, e.g., ultrasonic depth sensors.

In some embodiments, the bottom of the cap storage hopper (270) is angled at an angle a3, which can help the caps (205) load onto the cleats (230) properly. As an example, in some embodiments angle a3 is 60 degrees. In some embodiments, angle a3 is from 40 to 70 degrees. In some embodiments, angle a3 is from 20 to 70 degrees. The present invention is not limited to the aforementioned values for angle a3. In some embodiments, the bottom of the cap storage hopper (270) is constructed from a smooth material, e.g., smooth sheet metal, to assist in the flow of the caps (205) and/or positioning of the caps (205) where the cleats (230) enter the cap storage hopper (270). In some embodiments, the cleat enters the hopper at a particular position such that it only lifts vertically once inside the cap storage hopper (270), so as to avoid pushing the caps (205) back and away which would prevent proper loading of the caps (205) onto the cleat.

In some embodiments, the system (200) comprises a reject return panel (290) for helping to direct caps back to the storage hopper (270) that were fed incorrectly and identified as being wrong by the sensor (286).

The delivery guide (280), or gravity guide, functions to accept caps from the belt (220) and deliver them to the cap pickup location where they will be obtained by the capping robot. The delivery guide (280) may be constructed to allow gravity to cause the cap to rotate once it exits the belt feeder (210). For example, a cap may exit the belt feeder and the cap rotates such that it becomes positioned with its open end facing downward. The rotation may be approximately 100 degrees if, for example, the cap may leave the belt feeder at an 80 degree angle with respect to horizontal with the open end facing upward (equivalent to the cap leaning back 10 degrees) and be rotated forwardly 100 degrees until the open end faces downward. The amount of rotation will depend on the angle the cap originates in and the final angle of the cap with respect to horizontal. In some embodiments, the delivery guide (280) features a chute through which the cap travels from the belt feeder to the cap pickup location (288). In certain embodiments, the chute (282) is adapted to house several caps (e.g., up to 10, up to 12, more than 12, etc.) that are in line to the cap pickup location (288). The chute (282) may be configured to allow gravity to cause the caps in the chute to automatically move from within the chute to the cap pickup location (288). In some embodiments, the system stops the belt feeder (210) if there are too many caps in the chute (282) waiting for the cap pickup location. There may be a sensor for detecting the number of caps in the chute (282) waiting for the cap pickup location, which may trigger the on/off function of the belt feeder (210).

In some embodiments, the delivery guide (280) comprises a first sensor (284) for detecting whether or not a cap is present at the cap pickup location (288). In some embodiments, the delivery guide (280) comprises a second sensor (286) for detecting whether or not the cap is oriented appropriately. The system (200) may comprise a mechanism for retrieving a cap if it is not oriented properly, and it may be returned to the hopper (270). In some embodiments, the capping robot is instructed to return the cap to the hopper (270) with the reject return panel (290).

The cap feeding system described herein was designed to fit in a compact space within the frame of the automated system. The cap feeding system allows the cap to exit the belt feeder at a height sufficient to allow the use of gravity to reorient the caps and deliver the caps to the cap pickup location (for pickup by the capping robot). The height the caps exit the belt feeder also allows the delivery guide to hold a certain number of caps (e.g., 10 caps or other appropriate number), acting as a buffer for the capping robot.

### Bottle Feeding System

The bottle feeding system is configured to store bottles said and deliver bottles to the location where the bottles are capped. The bottles are delivered in the appropriate orientation and at the appropriate frequency.

To aid in efficiency and ease of use, the bottle feeding system may be designed with the following considerations. The bottle feeding system may be capable of processing bottles when the automated system is running, and the bottle storage hopper may be refillable without interfering with dispensation or processing of order. The bottle feeding system may have a large capacity to help reduce the frequency it needs to be reloaded. For example, the bottle feeding system may be capable of storing up to 700, up to 800, up to 900, up to 1,000, or 1,000 or more bottles. In certain embodiments, the bottle feeding system features a sensor for detecting when the bottle storage hopper is low, and the sensor may trigger an alarm or other alert device to indicate to a user that the bottle storage hopper should be refilled.

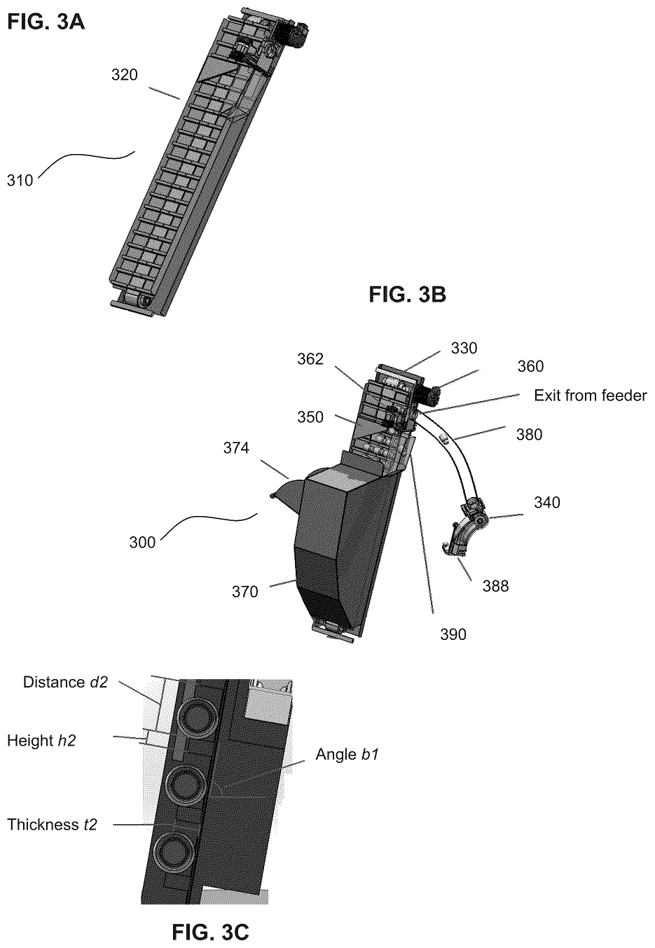

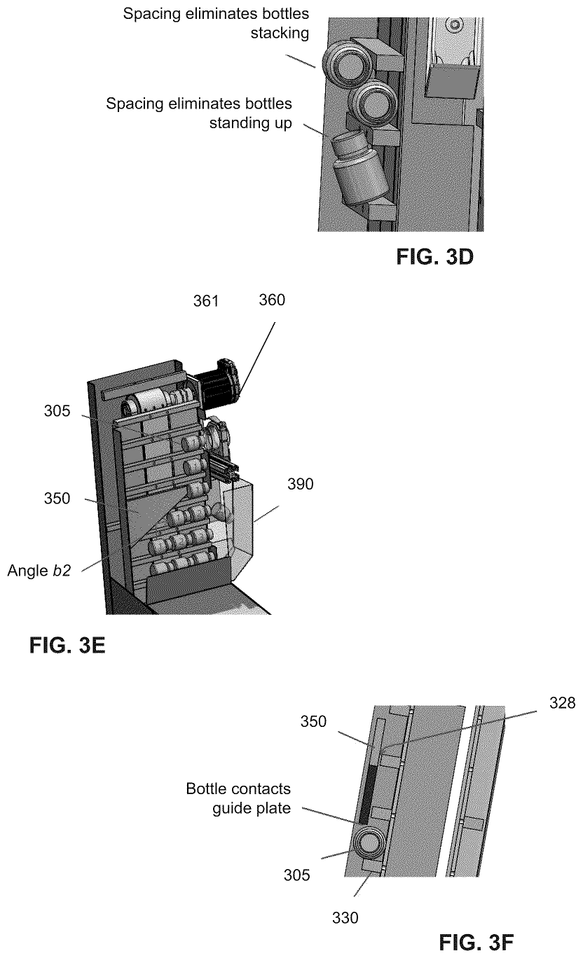

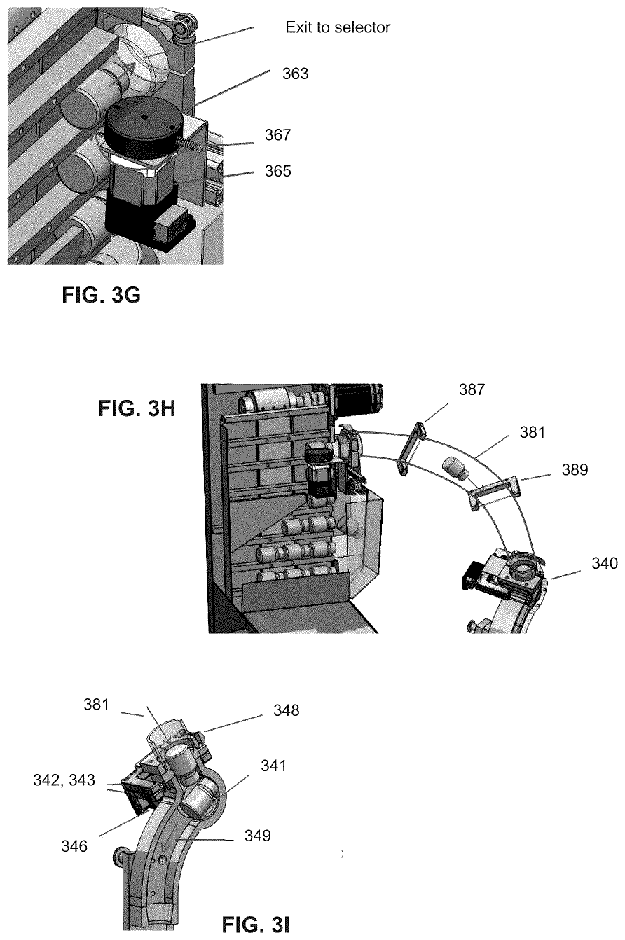

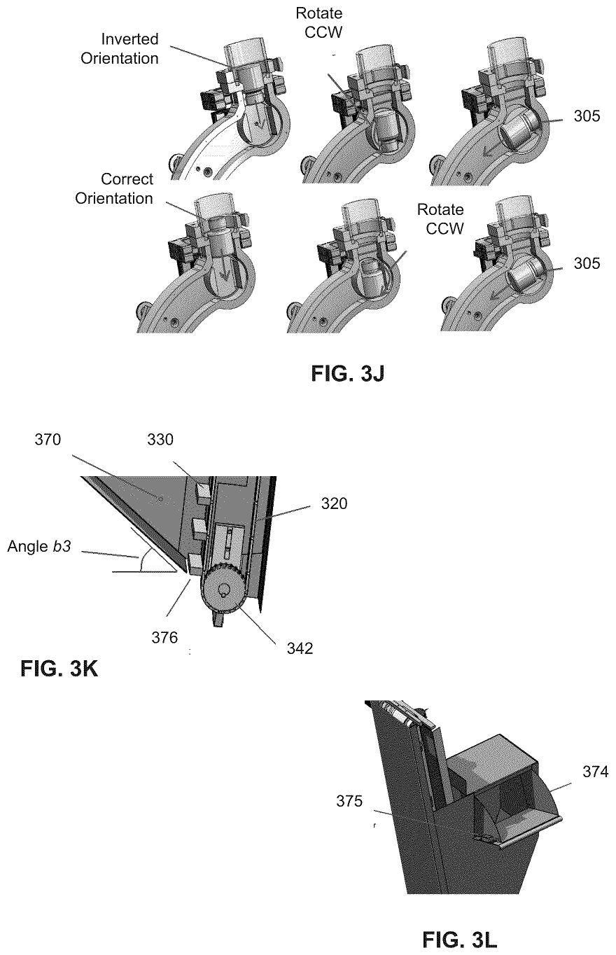

The present invention describes a particular bottle feeding system (300) shown in FIG. 3A, FIG. 3B, FIG. 3C, FIG. 3D, FIG. 3E, FIG. 3F, FIG. 3G, FIG. 3H, FIG. 3I, FIG. 3J, FIG. 3K, and FIG. 3L. The bottle feeding system described herein was designed to fit in a compact space within the frame of the automated system. It is understood that one of ordinary skill in the art may make modifications to the bottle feeding system depending on the bottle size, weight distribution, delivery frequency, angle of the belt feeder, etc., and said modifications are within the scope of the present invention.

The bottle feeding system (300) shown in FIG. 3A, FIG. 3B, FIG. 3C, FIG. 3D, FIG. 3E, FIG. 3F, FIG. 3G, FIG. 3H, FIG. 3I, FIG. 3J, FIG. 3K, and FIG. 3L comprises a bottle storage hopper (370) for housing and storing bottles, a belt feeder (310) for capturing bottles in the bottle storage hopper (370), and a bottle delivery guide (380) for delivering bottles from the belt feeder (310) to a bottle pickup location (388).

The belt feeder (310) comprises a belt (320) that rotates around an elongated plate or elongated axis formed by a drive gear (361), operatively connected to a drive motor (360), and a belt wheel (342). A plurality of cleats (330) is disposed on the belt (320) and spaced a distance apart. In some embodiments, the belt feeder (310) is designed to feed one bottle per cleat to the bottle pickup location (388). The present invention is not limited to this configuration.

For example, in some embodiments, a guide plate (350) is disposed at or near the top end of the belt feeder (310). The guide plate (350) is a mechanism for moving (or causing movement of) the bottles to the side of the belt feeder (310). In some embodiments, the guide plate (350) functions to allow only one bottle per cleat to reach the exit point of the belt feeder (110). For example, as bottles progress up the belt (320) on a cleat (330), bottles contacting the guide plate (350) will be slid toward the side of the belt and/or off of the belt into a return chute (390) that returns the bottles to the bottle storage hopper (370). The guide plate (350) may be positioned with respect to the belt such that one bottle can pass by the guide plate (350) without being pushed to the return chute (390). This process is shown in FIG. 3E.

Bottles that pass the guide plate (350) are pushed off of the belt (320) via a pusher wheel system (362) comprising a rotating pusher wheel (363) operatively connected to a pusher wheel motor (365), for example. Bottles pushed off are sent through the bottle delivery guide (380) to the bottle pickup location (388). In some embodiments, the pusher wheel comprises a flexible drive spring (367). In some embodiments, the rotating pusher wheel (363) has two springs on opposite sides. These springs may be fixed to the wheel and protrude to contact the bottle, regardless of the bottle orientation. The springs help progress the bottle into the delivery guide as the cleat progresses the bottle past the rotating pusher wheel (363). In some embodiments, the speed of the drive motor (360) and pusher wheel system (362) can be adjusted to accommodate a particular rate of bottle delivery.

The cleats (330) are spaced a distance d2 apart from each other. The cleats each have a particular width w2, as measured from the first side edge at or near the belt to the second side edge at or near the belt; the width w2 may be similar to the width of the belt (320), and a particular thickness t2, (e.g., depth). The cleats each have a particular height h2. The belt (320) is oriented at an angle b1 with respect to horizontal. The particular angle may be determined based on the size of the bottles, the weight of the bottles, and the weight distribution of the bottles.

As an example, the parameters of the bottle feeding system (300) may be designed such that the bottles (305) move up the belt feeder (310) with the open end facing to one side of the belt or the other side (e.g., see FIG. 3E). In certain embodiments, the parameters are configured to cause bottles that are not oriented properly to fall off back into the storage hopper (370), as shown in FIG. 3D. For example, as shown in FIG. 3D, the spacing of the cleats prevents bottles from resting on the cleats if they are standing up and prevents stacking of bottles.

In certain embodiments, angle b1 is 80 degrees. In certain embodiments, angle b1 is from 50 to 85 degrees. In certain embodiments, angle a1 is from 30 to 88 degrees. In certain embodiments, angle b1 is from 50 to 85 degrees. In certain embodiments, angle b1 is from 60 to 85 degrees. In certain embodiments, angle b1 is from 70 to 85 degrees. In certain embodiments, angle b1 is from 70 to 82 degrees. In certain embodiments, angle b1 is from 75 to 85 degrees. In certain embodiments, angle a1 is from 50 to 85 degrees. The present invention is not limited to the aforementioned angle values. The angle may vary depending on the bottle size, weight, and weight distribution, among other things.

In certain embodiments, distance d2 is 1-2 inches, e.g., 1.875 inches. In certain embodiments, distance d2 is from 1-3 inches. In certain embodiments, distance d2 is from 45 to 65 mm. In certain embodiments, distance d2 is from 1.5 to 3 inches. In certain embodiments, distance d2 is from 1 to 4 inches. The present invention is not limited to the aforementioned distance values. The distance may vary depending on the bottle size, weight, and weight distribution, among other things.

In certain embodiments, width w2 is 9.5 inches. In certain embodiments, width w2 is from 3 to 6 inches. In certain embodiments, width w2 is from 5 to 10 inches. In certain embodiments, width w2 is from 3 to 12 inches. In certain embodiments, thickness t2 is 0.875 inches. In certain embodiments, thickness t2 is from 0.1 to 1 inch. In certain embodiments, thickness t2 is from 0.5 to 2 inches. In certain embodiments, thickness t2 is from 0.5 to 3 inches. In some embodiments, the height h2 is 0.5 inches. In some embodiments, the height h2 is from 0.1 to 1 inch. In some embodiments, the height h2 is from 0.2 to 2 inches. The present invention is not limited to the aforementioned width, height, and thickness values, as they may vary depending on the bottle size, weight, and weight distribution, among other things, e.g., reduction in noise (e.g., the cleat in the hopper), etc.

The guide plate (350) may be constructed as a surface having an angle b2. In some embodiments, angle b2 is 30 degrees. In some embodiments, angle b2 is from 10 to 60 degrees. In some embodiments, angle b2 is from 20 to 70 degrees. The guide plate (350) is not limited to the particular angle described herein. The guide plate (250) is not limited to the configuration shown and described herein.

The cleats (330) may be constructed from low resistance material to help allow the bottles to slide on the cleat towards the bottle delivery guide (380) when contacted by the guide plate (350). A clearance (328) may be present between the cleats (330) and the guide plate (350) such that the bottles (but not the cleats (330)) contact the guide plate (250).

The bottle storage hopper (370) provides an opening (376) through which the cleats (330) can pass. The opening (376) provides adequate clearance for the cleats (330). The opening (376) is generally smaller than the bottles (305) to prevent the bottles (305) from inadvertently passing through the opening (376) and/or jamming the system. The bottle storage hopper (370) has a fill opening (374) (e.g., door) providing access to the inner cavity of the bottle storage hopper (370) to allow for refilling of the cap storage hopper (370). In some embodiments, the fill opening (374) comprises a sensor (375), e.g., for detecting if the fill opening is open thus necessitating shutting off of the belt feeder (310), as an example. In some embodiments, the hopper (370) can be refilled without the system having to stop. The fill opening (374) may be accessible via a refill door disposed in the frame (110) of the system (100). In some embodiments, the bottle storage hopper (370) comprises a sensor for detecting a level or amount of bottles (305) therein, In certain embodiments, the sensor is operatively connected to an alert component that is activated (thereby providing a user a signal) when the sensor detects too few bottles are in the storage hopper. Sensors configured to sense levels such as bottles in a storage unit are well known to one of ordinary skill in the art and include but are not limited to depth sensors, e.g., ultrasonic depth sensors.

In some embodiments, the bottom of the bottle storage hopper (370) is angled at an angle b3, which can help the bottles (305) load onto the cleats (330) properly. As an example, in some embodiments angle b3 is 60 degrees. In some embodiments, angle b3 is 65 degrees. In some embodiments, angle b3 is from 40 to 70 degrees. In some embodiments, angle b3 is from 20 to 70 degrees. The present invention is not limited to the aforementioned values for angle a3. In some embodiments, the bottom of the bottle storage hopper (370) is constructed from a smooth material, e.g., smooth sheet metal, to assist in the flow of the bottles (305) and/or positioning of the bottles (305) where the cleats (330) enter the bottle storage hopper (370).

The bottle delivery guide (380) functions to accept bottles from the belt (320) and deliver them to the bottle pickup location (388). As shown in FIG. 3H, FIG. 3I, and FIG. 3J, the delivery guide (380) comprises a gravity chute (381) connected to a selector (340). Gravity causes the bottles to travel through the chute (381) to the selector (340) at the end of the chute (381). In some embodiments, the chute (381) can hold a certain number of bottles (e.g., 5, 6, 7, 8-10, 6-12, etc.) as a buffer. In some embodiments, the delivery guide (380) comprises a first bottle sensor (387) in the chute (381) for detecting a backup in the number of bottles, causing the feeder system to turn off to eliminate overfilling of the chute. In some embodiments, the delivery guide (380) comprises a second bottle sensor (389) in the chute for detecting when there is adequate open space for bottles to be fed to the delivery guide (380), which would turn on the feeder system.

The bottle feeding system (300) may feature time delays. For example, sensors may be required to sense the presence or absence of a bottle for a certain amount of time before signaling to other parts of the system to help ensure an accurate read or detection event, e.g. avoid considering a bottle passing through the chute as detection of the presence or a bottle backup.

FIG. 3I and FIG. 3J show detailed views of the selector (340). Bottles at the end of the chute (381) enter the selector (340), e.g., a rotator wheel (341), which is operatively connected to a rotator wheel drive motor (346). The rotator wheel (341) functions to rotate bottles clockwise and counterclockwise depending on the orientation of the bottle. Once the bottle is in the correct orientation, e.g., with the opening facing upwardly, for example, the bottle is released from the rotator wheel (341) to the bottle pickup location (388) via a second chute (349), for example via gravity or another mechanism. The top panels of FIG. 3J show an inverted bottle entering the rotator wheel (341), and the bottom panels of FIG. 3J show an correctly oriented bottle entering the rotator wheel (341). The selector (340) further comprises a first selector sensor (342) for detecting if the bottle is in the correct position in the rotator wheel (341) and a second selector sensor (343) for detecting the orientation of the bottle (e.g., opening facing up or opening facing down). The sensors control the actions of the rotator wheel (341). In some embodiments, the selector (340) further comprises a quick release (348) to allow for accessing the chute (381) and/or selector (340) to help clear a jam, if necessary.

### Dial Table System

The dial table system is a multi-position system that provides the locations for allowing bottles to be filled and capped. For example, the dial table system accepts an empty bottle, positions the empty bottle under the medication dispenser, allows time for medication to be dispensed, positions the bottle to be capped, allows time for the bottle to be capped, and positions the bottle in an unloading position so it can be moved to the printing system. The system is designed to fill and cap bottles faster than by hand, e.g., at least 2 times as fast, at least 3 times as fast, at least 4 times as fast, etc. The dial table system is designed to be accessible within the frame (110) to allow for clearing jams, providing maintenance, for cleaning, or for disassembly.

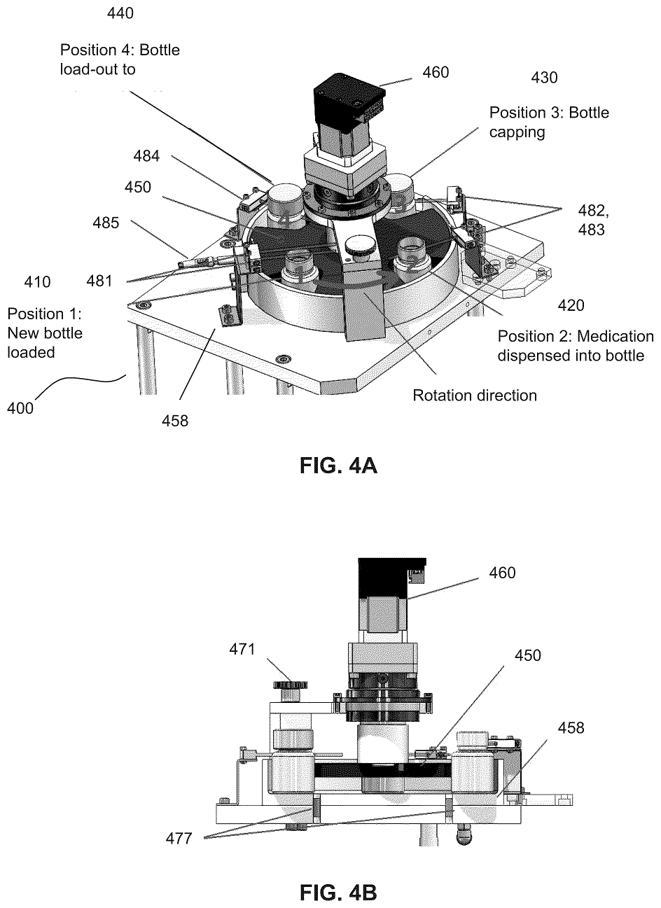

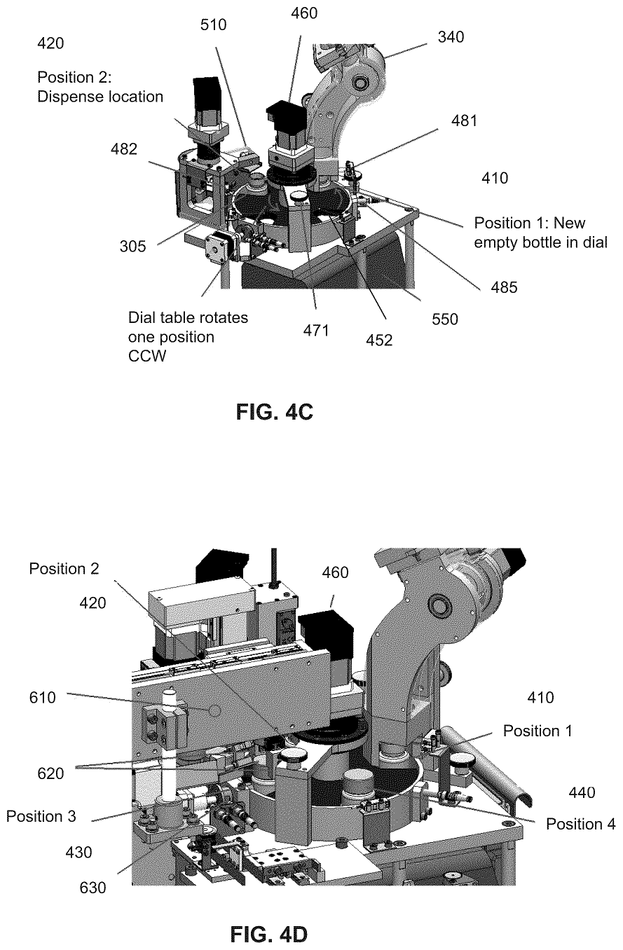

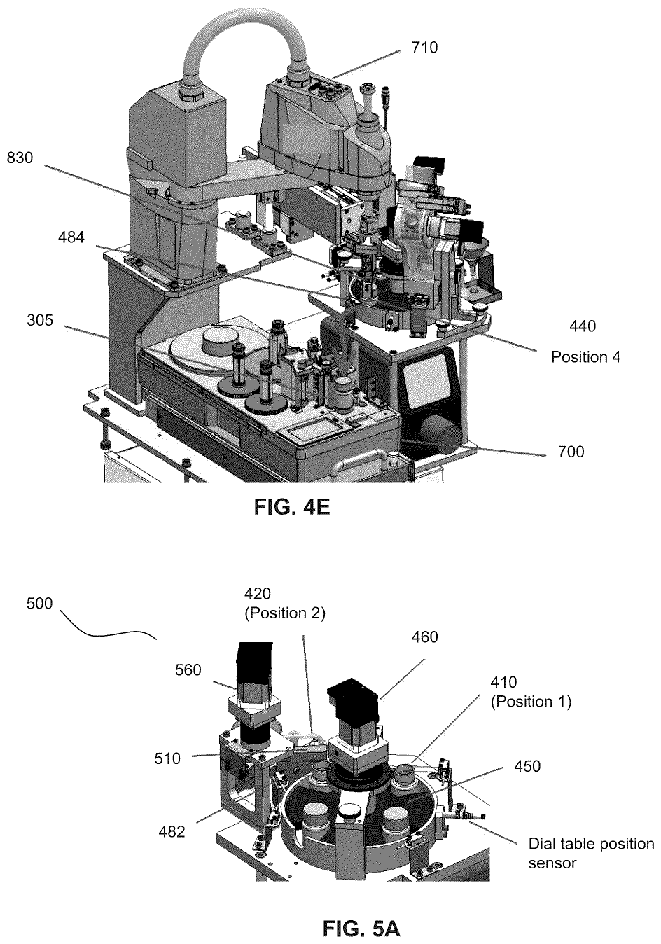

The present invention describes a dial table system (400) shown in FIG. 4A, FIG. 4B, FIG. 4C, FIG. 4D, and FIG. 4E. The dial table system (400) described herein was designed to fit in a compact space within the frame of the automated system (100). It is understood that one of ordinary skill in the art may make modifications to the dial table system (400) and said modifications are within the scope of the present invention.

The dial table system (400) shown in FIG. 4A, FIG. 4B, FIG. 4C, FIG. 4D, and FIG. 4E comprises a rotating dial (450), operatively connected to a dial motor (460). The rotating dial (450) comprises a plurality of slots therein adapted to accept a bottle, e.g., four slots (452). The rotating dial (450) may be rotatably connected to a platform (458), as shown, e.g. via the dial motor (460). The rotating dial (450) rotates, e.g., in a first direction, so as to occupy particular positions, e.g., to move bottles from one position to the next position. For example, the rotating dial (450) may rotate so a first slot occupies a first position (410), or loading position, wherein a bottle is loaded into the slot; a second slot occupies a second position (420), or dispensing position, wherein the bottle is positioned in relationship to the dispensing system such that medication is dispensed into the bottle; a third slot occupies a third position (430), or capping position, wherein the bottle is positioned in relationship to the capping system such that the bottle can be capped; and a fourth slot occupies a fourth position (440), or unloading position, wherein the bottle is positioned in relationship to the unloading robot such that the capped bottle can be unloaded from the slot, making the slot vacant. The present invention is not limited to the configuration described herein.

The rotating dial (450) is adapted to accept an empty bottle (with the open end facing upwardly) from the bottle feeding system (300), e.g., the bottle pickup location (388), wherein the empty bottles are inserted into the slot in the first position (410). In some embodiments, the system uses gravity to assist the bottle entering the slot. In some embodiments, the system comprises robotics for placing the bottle in the slot. Next, the rotating dial (450) rotates a first turn to move the bottle from the first position (410) to the second position (420) where it is filled with medication. Once the dispensing of the medication is complete, the rotating dial (450) rotates a second turn to move the bottle from the second position (420) to the third position (430) where it is capped. After capping, the rotating dial (450) rotates a third turn to move the bottle from the third position (430) to the fourth position (440) where it is unloaded from the dial table (450). After unloading, the rotating dial (450) rotates a fourth turn to move the bottle from the fourth position (440) to the first position (410) where it is loaded again with an empty bottle. In some embodiments, each turn is a 90 degree rotation of the dial table (450).

In some embodiments, the dial table system (400) further comprises a first sensor (481) for detecting if a bottle is present in the slot that is occupying the first position (410). In some embodiments, the dial table system (400) further comprises a second sensor (482) for detecting if a bottle is present in the slot that is occupying the second position (420). In some embodiments, the dial table system (400) further comprises a third sensor (483) for detecting if a bottle is present in the slot that is occupying the third position (430). In some embodiments, the dial table system (400) further comprises a fourth sensor (484) for detecting if a bottle is present in the slot that is occupying the fourth position (440). In some embodiments, the first sensor (481) or an additional sensor detects whether the bottle that is in the slot that is occupying the first position (410) is seated in the slot properly. In some embodiments, the system further comprises a dial table position sensor (485) for detecting the position of the rotating dial (450). If the sensors detect an error, the system may stop so that the problem can be addressed.

In some embodiments, the dial table system or components thereof are assembled with thumb screws (471) and/or locating pins (477) to allow for easy maintenance, cleaning, and assembly.

The dial table system (400) allows for the rapid filling of bottles. The rate of the rotation can depend on the actions occurring at particular positions of the dial table, e.g., how long the bottle takes to be filled with medication, how long the bottle takes to be capped, etc., since there is a dwell time for each process. As an example, the system (100) may function such that a dose can be dispensed in approximately 1 second. If the actions occurring at the other positions of the dial table take 1 second or less, then the dial table would rotate every 1 second. If the filling time is shorter than the time required for capping, then the rotation may be limited by the speed of capping. Note that the time required for dispensation may depend on the volume of medication being dispensed, e.g. not every bottle will require the same amount of filling time. In some embodiments, the dwell time at a position in the dial table is 1 second or less. In some embodiments, the dwell time at a position in the dial table is less than 1 second. In some embodiments, the dwell time at a position in the dial table is 1 millisecond. The present invention is not limited to the aforementioned dwell times. In some embodiments, the dwell time may be more than 1 second, e.g., 1.5 seconds, 2 seconds, 3 seconds, etc.

### Dispensing System

The dispensing system allows for medication to be dispensed into empty bottles positioned in the dial table system. As an example, in some embodiments, the dispensing system comprises a medication pump, tubing, a medication reservoir, and a dispenser. In some embodiments, the dispensing system comprises tubing, a medication reservoir, and a dispenser but not the medication pump, and the system is designed for easy integration of a medication pump.

To aid in efficiency and ease of use, the dispensing system may be designed with the following considerations. The dispensing system may be easily accessible in the frame of the automated system, e.g., the pump, the calibration flask, the medication reservoir, the maintenance, and cleaning fluids, etc., are easily accessible. The dispensing system allows for performing maintenance and calibration tasks. The dispensing system is positioned or is capable of positioning itself to avoid or reduce messes.

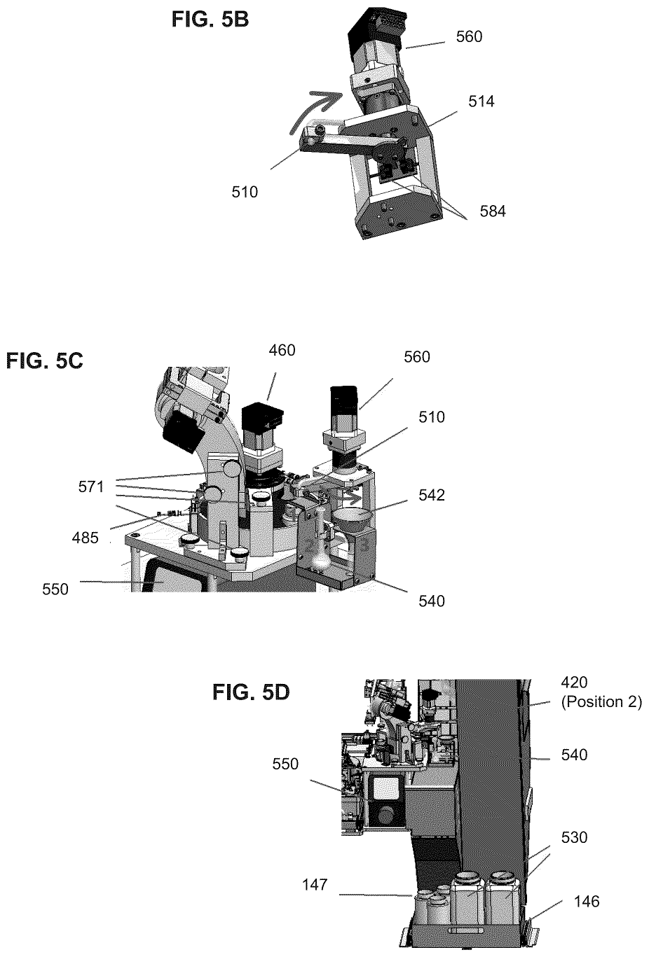

The present invention describes a dispensing system (500) shown in FIG. 5A, FIG. 5B, FIG. 5C, and FIG. 5D. The dispensing system (500) described herein was designed to fit in a compact space within the frame of the automated system (100). It is understood that one of ordinary skill in the art may make modifications to the dispensing system (500) and said modifications are within the scope of the present invention.

The dispensing system (500) shown in FIG. 5A, FIG. 5B, FIG. 5C, and FIG. 5D comprises a dispenser arm (510) through which medication is aliquoted into a medication bottle, e.g., via an exit port of the dispenser arm (510). The dispenser arm (510), e.g., exit port, is fluidly connected, e.g., via tubing, to a medication reservoir (530) which stores the medication. The medication may be pumped from the medication reservoir (530) through tubing via a medication pump (550). As previously discussed, in some embodiments, the dispensing system (500) includes a medication pump (e.g., standard of care medication pump). In some embodiments, the dispensing system (500) does not include the medication pump but is adapted for easy integration of a medication pump, e.g., a clinic's standard of care pump. Non-limiting examples of standard of care pumps include the Methaspense, Digispense, CSDS, and LabTec pumps.

Tubing may include but is not limited to a first tube having a first end fluidly connected to the medication reservoir (530) and a second end adapted to operatively connect to a medication pump (550) such that medication can be pumped from the medication reservoir (530). Tubing may also include a second tube having a first end fluidly connected to the dispenser arm (510) and a second end adapted to operatively connect to a medication pump (550) such that medication directed from the medication pump (550) can be dispensed via the dispenser arm (510).

The dispenser arm (510) can occupy one or a plurality of positions, and move between two or more positions. For example, in some embodiments, the dispenser arm (510) can occupy a first position (dispense position) wherein in the first position the dispenser arm (510) is positioned to dispense medication into a bottle held in the dial table system (400), e.g., in position 2 of the dial table system. The specific amount of medication that is dispensed into the bottle is determined by the clinic's electronic medical record. In some embodiments, the dispenser arm (510) can occupy a second position (calibration position) wherein in the first position the dispenser arm (510) is positioned to dispense a fluid into a calibration flask (540), which is for calibration processes e.g., the fluid in the calibration flask (540) may be measured for accuracy. In some embodiments, the dispenser arm (510) can occupy a third position (waste/maintenance position) wherein in the first position the dispenser arm (510) is positioned to dispense a fluid into a waste container or waste funnel (542), which is for accepting waste fluids. In some embodiments, the dispenser arm (510) can move from the first position to the second position. In some embodiments, the dispenser arm (510) can move from the first position to the third position. In some embodiments, the dispenser arm (510) can move from the second position to the third position. In some embodiments, the dispenser arm (510) can move from the second position to the first position. In some embodiments, the dispenser arm (510) can move from the third position to the second position. In some embodiments, the dispenser arm (510) can move from the third position to the second position. The present invention is not limited to three positions the dispenser arm (510) can occupy.

Movement of the dispenser arm (510) between two or more positions may be driven by the dispenser arm drive motor (560) operatively connected to the dispenser arm (510). The dispenser system (500) may further comprise one or more travel sensors (584) for determining whether or not the dispenser arm (510) is in a particular position, e.g., a first position, a second position, a third position, etc. In some embodiments, the sensor is operatively connected to an alert system designed to alert a user if the dispenser arm (510) is not aligned properly in position. In some embodiments, the dispenser arm (510) comprises travel tabs (514), which are extensions or protrusions that help the dispenser arm (510) occupy the desired position without moving out of place. In some embodiments, the sensors work in combination with the travel tabs (514) to ensure proper placement of the dispenser arm (510).

The dispenser arm (510) may be positioned next to or in proximity to the dial table system (400). In some embodiments, the dispenser arm (510) is operatively connected to the dial table system (400). As shown in FIG. 5C, the dispenser arm (510) may be constructed to be integrated with the dial table system (400) via connectors such as but not limited to thumb screws (571).

As will be discussed herein, the dispensing system (500) (e.g., the drive motor (560), the pump (550), etc.) receives instructions from the control center. Instructions received from the control center may include but are not limited to: (i) pump medication to a reservoir to empty the pump; (ii) pump medication to prime the tubing; (iii) pump cleaning solution to clean the tubing and pump; (iv) pump distilled water to flush the tubing and pump after the cleaning cycle or prior to storing the pump; (v) pump distilled water into the waste container; (vi) pump medication for calibration.

The fluids such as but not limited to fluids in the waste container or calibration fluids may be housed in reservoirs seated on a fluid drawer (146). The fluid drawer (146) may be easily accessible to personnel for refilling or emptying purposes, e.g., via a drawer.

### Capping System

The capping system is the system that obtains caps and caps the bottles in a secure manner. To aid in efficiency and ease of use, the capping system may be designed with the following considerations. The capping system may be easily accessible in the frame of the automated system. The capping system allows for fast, secure, and efficient capping of bottles, including the use of child-proof caps. The capping system may also allow for poorly capped bottles to be moved to a separate location (e.g., for review) and allow the automated system (100) to recover and produce a replacement dose for the order.

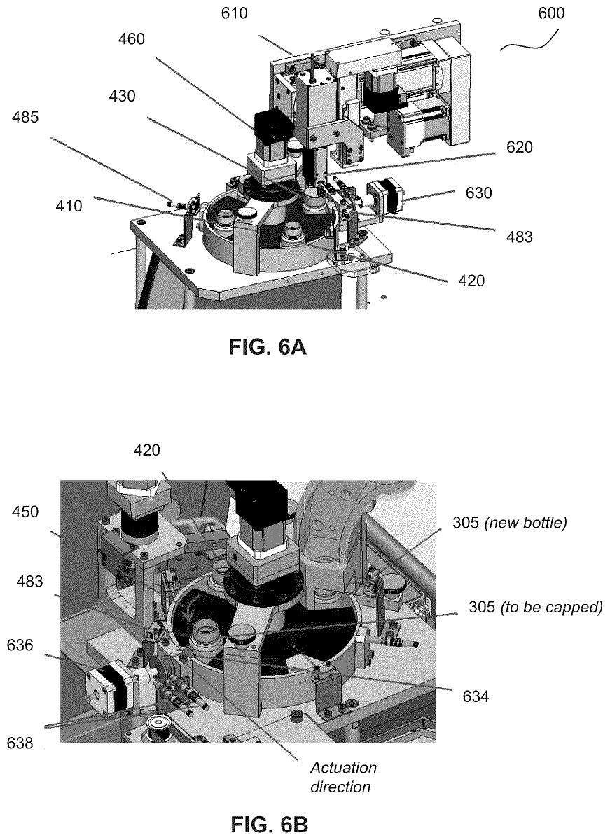

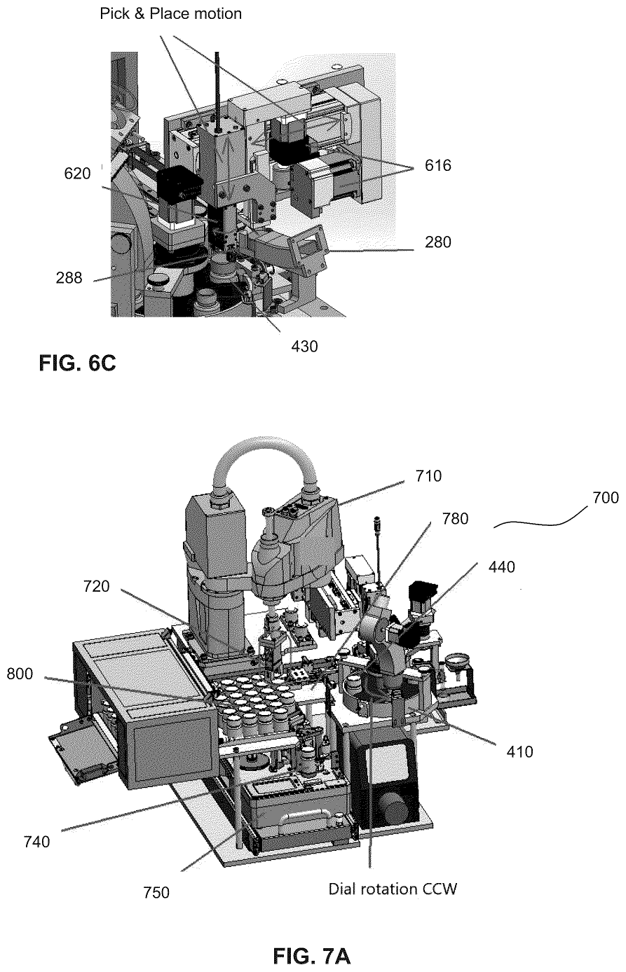

The present invention describes a capping system (600) shown in FIG. 6A, FIG. 6B, and FIG. 6C. The capping system (600) described herein was designed to fit in a compact space within the frame of the automated system (100). It is understood that one of ordinary skill in the art may make modifications to the capping system (600) and said modifications are within the scope of the present invention.

The capping system (600) shown in FIG. 6A, FIG. 6B, and FIG. 6C comprises a pick and place system (610) which functions to obtain caps and attach the caps to bottles when the bottles are in the appropriate position of the dial table system (400) (e.g., position 3 (430)); and a bottle clamp system (630) for holding the bottle in place and preventing movement or rotation of the bottle during the capping process. In some embodiments, a rotary gripper (620) performs the function of retrieving a cap from the cap pickup location (288) and threading the cap on the bottle. For example, the pick and place system (610) comprises motors (616) operatively connected to the rotary gripper (620) for moving the rotary gripper (620), e.g., between various positions such as from the cap pickup location (288) to the bottle to be capped, for rotating the rotary gripper (620) so as to cap a bottle, etc.

The bottle clamp system (630) comprises a bottle clamp cylinder (634) and a bottle clamp motor (636) operatively connected to the bottle clamp cylinder (634). The bottle clamp motor (636) drives motion of the bottle clamp cylinder (634), e.g., between an engaged position and a disengaged position. In the engaged position, the bottle clamp cylinder (634) clamps and immobilizes the bottle securely in position without damage (e.g., while the pick and place system (610) attaches a cap to the bottle). When the bottle clamp cylinder (643) is in the disengaged position, the bottle is no longer immobilized, and the dial table system (400) can rotate.

In some embodiments, the bottle clamp cylinder (634) comprises a gripping component (such as but not limited to rubber) to help provide a grip on the bottle for immobilization. In some embodiments, the bottle clamp system (630) comprises one or more bottle clamp position sensors (638) for detecting the position of the bottle clamp cylinder (634).

Once the bottle is immobilized by the bottle clamp cylinder (634), the pick and place system (610) is activated to cap the bottle. The rotary gripper (620) of the pick and place system (610) may already be waiting directly over the bottle with a cap. The rotary gripper (620) may comprise a torque feedback system to help aid controlled thread engagement between the cap and bottle. When activated, the pick and place system (610) lowers the cap via the rotary gripper (620) to a specific distance above the bottle, and the rotary gripper (620) begins rotating and lowering the cap at a specific rate so as to allow the cap's thread to engage the bottle properly. Once a certain torque value and distance are reached by the rotary gripper (620), e.g., when the cap is securely engaged on the bottle, the rotary gripper (620) stops rotating, disengages from the cap, and then retracts.

In some embodiments, if the expected values for the torque and distance aren't achieved (e.g., as detected by the torque feedback system of the rotary gripper (620)), the capping is not considered successful and the bottle will be placed in a different location.

In some embodiments, the pick and place system is configured to continuously retrieve a cap and return to the ready position over a bottle in preparation for capping. For example, in some embodiments, after capping a bottle, the pick and place system will immediately move to pick up another cap from the cap pickup location (288) and return to the ready position over the next bottle to save time.

The capping system (600) may further comprise a sensor for detecting whether or not the bottle is in the proper position in order to be capped. In some embodiments, the capping system (600) may further comprise a sensor for detecting whether or not the cap is attached properly. In some embodiments, this function is achieved via the rotary gripper (620), e.g., by determining whether or not the expected values for torque and distance are achieved. In some embodiments, the capping system (600) is configured to divert poorly capped bottles to a separate location, e.g., a quarantine location (780), or configured to alert other components of the system that the bottle needs to be diverted to the quarantine location (780).

### Labeling System

The labeling system is the system that obtains a bottle from the dial table system (400), e.g., position 4 (440), labels the bottles, and either places the bottle in the order buffer or a quarantine location (780) if the label is unreadable, the dose is incorrect or the capping is poor. To aid in efficiency and ease of use, the labeling system may be designed with the following considerations. The labeling system may be easily accessible in the frame of the automated system. The labeling system is configured to allow for rapid dose and order assembly. In some embodiments, the labeling system helps divert doses with problems and allow for the system (100) to remake the dose for the order.

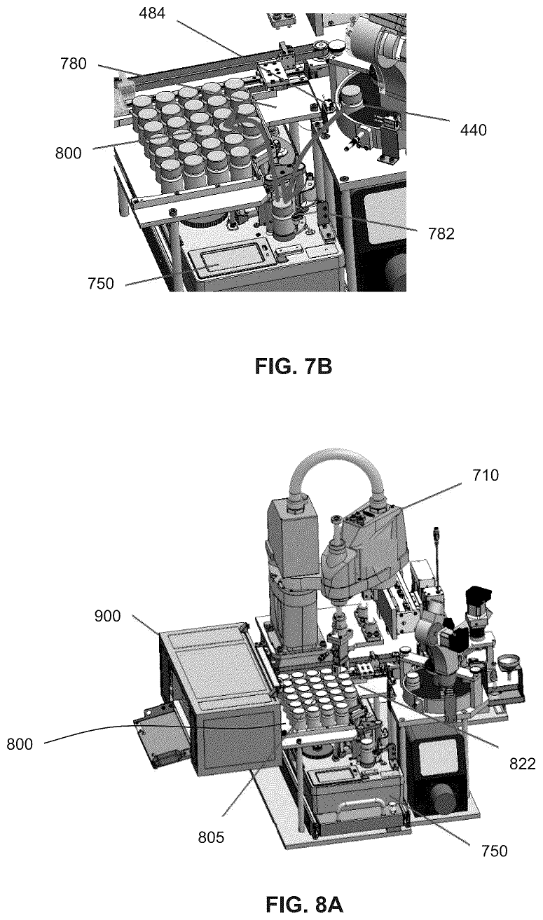

The present invention describes a labeling system (700) shown in FIG. 7A and FIG. 7B. The labeling system (700) described herein was designed to fit in a compact space within the frame of the automated system (100). It is understood that one of ordinary skill in the art may make modifications to the labeling system (700) and said modifications are within the scope of the present invention.

The labeling system (700) shown in FIG. 7A and FIG. 7B comprises a robotic arm system (710), e.g., SCARA robot, for obtaining and moving bottles between various positions, e.g., from the dial table (440) to a printing position (740) for labeling, from the printing position to the order buffer or quarantine location, etc.; and a printing system (750) for printing and applying labels on the bottles and monitoring the labels thereon.

The robotic arm system (710) comprises a gripper (720) (e.g., with jaws) that can move to the bottle pickup position in order to pick up the bottle from the dial table (440) out of position 4. The robotic arm system (710) can move the gripper (720) from the bottle pickup position to the printing position (740) of the printing system (750), whereupon the gripper (720) of the robotic arm system (710) releases the bottle to allow the bottle to be labeled by the printing system (750). In some embodiments, the robotic arm system (710), e.g., gripper (720), may hover in the printing position (740) while the bottle is labeled and reviewed by the printing system (750). When activated, the robotic arm system (710) may then move the gripper (720) to retrieve the bottle from the printing system (750), move the gripper (720) to the order buffer position where it releases the bottle in the order buffer system (if the label is correct), or move the gripper (720) to the quarantine position where it releases the bottle in the quarantine location (780) (if the label is incorrect or compromised). After delivery of the bottle, the gripper (720) is moved back to the bottle pickup position in order to pick up a bottle from position 4 of the dial table (440).

The printing system (750) comprises a printer and a label application machine. In some embodiments, the printing system (750) comprises a sensor (782) for detecting whether or not a bottle is in the printing position (740). When the bottle is properly placed, the label application machine of the printing system (750) applies the printed label associated with that particular bottle to the exterior of the bottle. In some embodiments, the label has a standard barcode printed thereon. In some embodiments, the printing system (750) comprises a scanner for scanning the barcodes on the labels to ensure the label is accurate before the bottle is delivered to the order buffer system (800). As previously discussed, if the label is compromised, e.g., if the barcode cannot be read or is incorrect, the bottle will be transferred to the quarantine location.

Referring to FIG. 7C, in some embodiments, the label (306) features a tab (307) extending from an edge of the label (306) but removable via perforations or other destructive mechanisms. The tab (307) is for attaching to the cap (205) while the label (306) is attached to the bottle (305). This provides a mechanism for detecting whether or not the bottle (305) has been opened after it is capped. For example, if a user twists off the cap (205), the tab (307) is permanently disconnected from the label (306), thus indicating the cap (205) has been removed from the bottle (305) at least once. There may be a barcode on the label that becomes unscannable once the bottle is opened. In some embodiments, the label overlaps the cap.

The system (100) such as but not limited to the labeling system (700), e.g, printing system (750), label application machine, etc. may be constructed or modified to accommodate the type and/or size of label being printed and applied to the bottle. For example, if the label is sufficiently larger and requires larger caps and/or bottles, other components of the system (100) such as the cap feeding system and/or bottle feeding system may be modified to accommodate the different caps and/or bottles. In some embodiments, the label application machine may comprise different sizes or numbers of rollers depending on the label size and configuration.

### Order Buffer System

The order buffer system is the system that assembles orders and stores completed orders if they cannot be retrieved immediately. To aid in efficiency and ease of use, the order buffer system may be designed with the following considerations. The order buffer system may be easily accessible in the frame of the automated system. The order buffer system is configured to support rapid and accurate dose assembly. For example, in order to provide rapid order processing, the order buffer system allows for continuation of order fulfillment even if a previous order has not been retrieved.

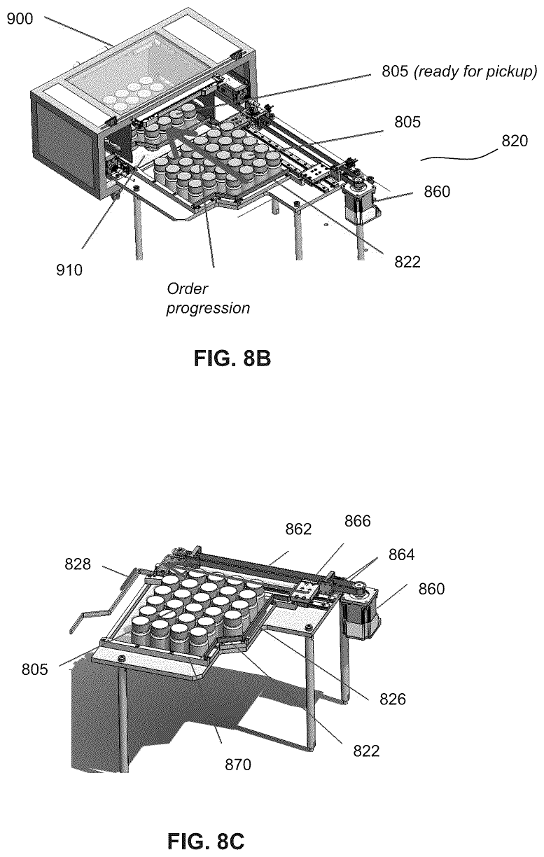

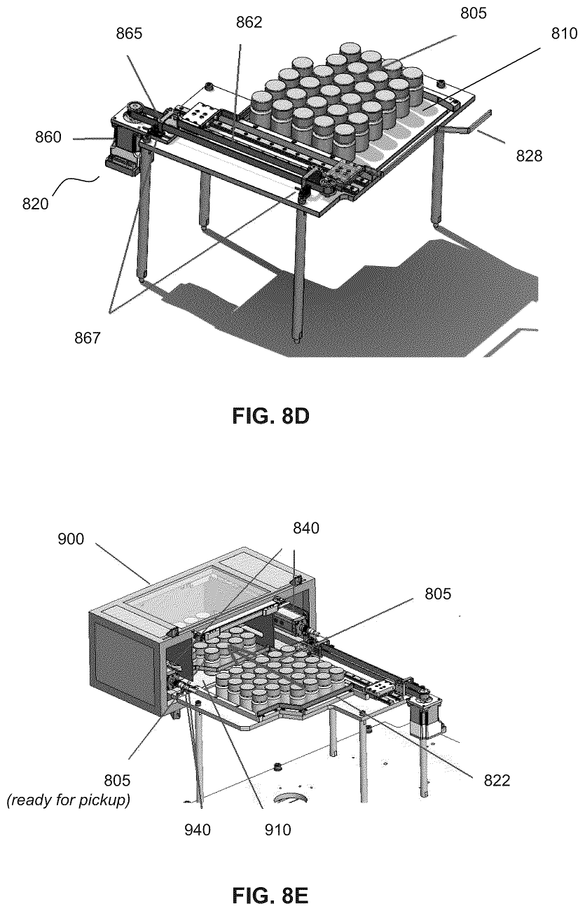

The present invention describes an order buffer system (800) shown in FIG. 8A, FIG. 8B, FIG. 8C, FIG. 8D, and FIG. 8E. The order buffer system (800) described herein was designed to fit in a compact space within the frame of the automated system (100). It is understood that one of ordinary skill in the art may make modifications to the order buffer system (800) and said modifications are within the scope of the present invention.

The order buffer system (800) is operatively linked with the labeling system (700) and the tray drawer system (900), which is the access point where healthcare professionals retrieve orders. For example, the order buffer system (800) may be positioned adjacent to the tray drawer system (900) such that filled orders in the order buffer system (800) easily travel to the tray drawer system (900). As doses are completed for a specific order, they are moved from the labeling system (700), e.g., the printing system (750), to the order buffer system (800) by the robotic arm system (710) as an organized array (805) of bottles. In some embodiments, the order buffer system (800) has the capacity to hold at least 28 bottles or doses, however the present invention is not limited to this configuration. In some embodiments, the order buffer system (800) has the capacity to hold or buffer more than one order.

The order buffer system (800) shown in FIG. 8A, FIG. 8B, FIG. 8C, FIG. 8D, and FIG. 8E comprises an array platform (810), which is the platform on which filled bottles are placed by the robotic arm system (710) of the labeling system (700). The array platform (810) is configured to accept a plurality of filled bottles, creating an array (805). The array platform (810) may feature one or more guide rails (870) for helping to prevent the bottles in the array (805) from falling off of the array platform (810). The order buffer system (800) further comprises a mechanism for moving the array (805) to the tray drawer system (900) (e.g., the tray (910) in the tray drawer (920) of the tray drawer system (900)) for retrieval. For example, in some embodiments, the order buffer system (800) comprises a pusher arm system (820) for pushing the array (805) from the array platform (810) to the tray (910) of the tray drawer system (900). The pusher arm system (820) may comprise an elongated arm (822) operatively connected to a motor (860), which drives movement of the elongated arm (822) between at least two positions, e.g., a first position (826) wherein the elongated arm (822) is retracted behind the array (805), and a second position (828) wherein the elongated arm (822) has moved toward the tray (910) of the tray drawer system (900) sufficiently to move the array (805) completely onto the tray (910). As an example, the pusher arm system (820) may further comprise a drive belt (862) operatively connected to the motor (860) and rotatable around an axis, wherein the motor (860) drives rotation of the drive belt (862) about the axis in a first direction and a second direction opposite the first. The pusher arm system (820) may further comprise a support carriage (866) operatively connected to the drive belt (862) and operatively connected to the elongated arm (822), such that rotation of the drive belt (862) via the motor (860) causes the support carriage (866) and elongated arm (822) to move accordingly, e.g., from the first position (826) to the second position (828) and vice versa. In some embodiments, the support carriage (866) rides on carriage guide rails (864) that stabilize the elongated arm (822) as it progresses between at least the first position (826) and the second position (828).

The tray drawer system (900) is configured to accommodate the pusher arm system (820) to allow movement of the array (805) into the tray drawer (920) and onto the tray (910). Once the array (805) has been pushed to the tray (910), the pusher arm system (820) retracts to be available for the next array (805), and the robotic arm system (710) of the labeling system (700) can begin again.

In some embodiments, the pusher arm system (820) further comprises an elongated arm position flag (865), which functions to trigger arm sensors (867) that can detect if the elongated arm (822) is out of position, e.g., has traveled too far, etc.

In some embodiments, the order buffer system (800) or tray drawer system (900) comprises one or more sensors for providing feedback about the status of the array (805). For example, the order buffer system (800) may comprise a sensor for determining the position of the elongated arm (822). In some embodiments, the order buffer system (800) comprises sensors for determining the array is ready to be moved by the pusher arm system (820). In some embodiments, the order buffer system (800) or tray drawer system (900) comprises a clearance sensor (840), e.g., a light curtain, e.g., for viewing the tray (910) for determining if the tray drawer is clear of bottles. If a bottle remains on the tray (910), the pusher arm system (820) will not be allowed to move the next array into the tray drawer (920).

The present invention is not limited to the configuration that stores 1 completed array in the order buffer system while another array of medication bottles is filled and assembled. For example, in some embodiments, the system comprises an array holding system, that is capable of storing, organizing, and/or retrieving a plurality of completed arrays that may be waiting to be picked up while other arrays are organized. The array holding system may be an internal or external feature of the automated system. As a non-limiting example, the array holding system may comprise a plurality of racks stacked in a particular manner that allows for the system to place and remove trays (with completed arrays) thereon.

### Tray Drawer System

The tray drawer system is the system that accepts completed orders in the form of an order array (805), as previously described, and provides healthcare professionals access to the order array. To aid in efficiency and ease of use, the tray drawer system may be designed with the following considerations. The tray drawer system may be easily accessible in the frame of the automated system. The tray drawer system is configured to allow easy and fast order pickup. The tray drawer system may notify the appropriate healthcare professional that his/her order is ready. The tray drawer system is also adapted to work with the order buffer system to allow for holding of an order if the order in the tray drawer system cannot be picked up right away.

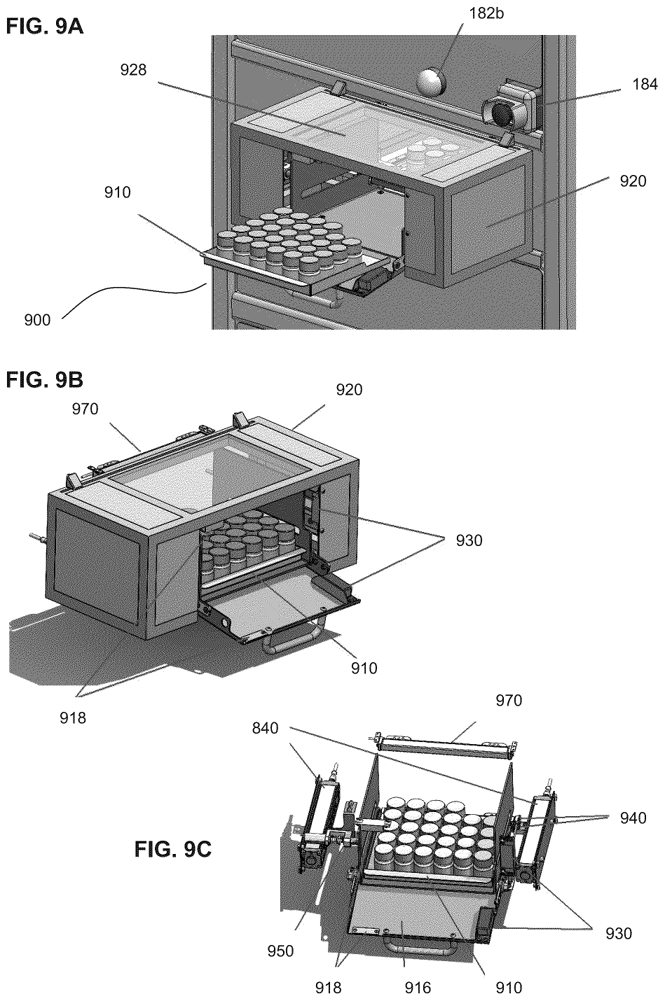

The present invention describes a tray drawer system (900) shown in FIG. 9A, FIG. 9B, and FIG. 9C. The tray drawer system (900) described herein was designed to fit in a compact space within the frame of the automated system (100). It is understood that one of ordinary skill in the art may make modifications to the tray drawer system (900) and said modifications are within the scope of the present invention.

The tray drawer system (900) shown in FIG. 9A, FIG. 9B, and FIG. 9C comprises a tray drawer (920) with an inner cavity adapted to accept an order array (805). In some embodiments, the tray drawer (920) extends from the frame (110) of the automated system (100), e.g., as shown in FIG. 9A.

A door (916) is disposed in the tray drawer (920) which can move between at least an open position, which provides access to the inner cavity of the tray drawer (920) (e.g., shown in FIG. 9A), and a closed position, which is when the door (916) prevents access to the inner cavity of the tray drawer (920). In some embodiments, the door (916) operates via a hinge, however the present invention is not limited to this configuration. In some embodiments, the tray drawer (920) comprises a transparent area (928), e.g., in the top of the tray drawer (920), for providing visual access to the tray drawer (920) so a healthcare professional can see if an order is in the tray drawer (920). Disposed in the tray drawer (920) is a removable tray (910), which is the surface on which the order arrays (805) are placed while awaiting pickup. The tray (910) may be obtained from the tray drawer (920) when the door (916) is in the open position.

In some embodiments, the door (916) can be temporarily secured in the closed position via a closure component (918) such as but not limited to a magnet or a pair of magnets. In some embodiments, the tray drawer system (900) comprises a tray door sensor (930) for determining whether or not the drawer (916) is in the open position or closed position. In some embodiments, the tray drawer system (900) further comprises a door lock (950) that can prevent the door from moving from the closed position to the open position. In some embodiments, the door lock (950) is operated by a solenoid. In some embodiments, the door must be in the closed position and locked in order for the next array to be moved to the tray drawer system (900).

In some embodiments, the tray drawer system (900) comprises a light curtain (841), e.g., for viewing the inner cavity of the tray drawer (920), e.g., the tray (910) or the space therein, for determining if the tray drawer (920) is clear of bottles. If a bottle remains on the tray (910), the clearance sensor (840) can send a signal to the other components of the system to prevent the pusher arm system (820) from moving the next array into the tray drawer (920).

In some embodiments, the tray drawer system (900) comprises tray sensors (940) in the tray drawer (920). These sensors (940) may function to determine if the tray (910) is in the correct position and/or if there is more than one tray (910) present in the tray drawer (920). In some embodiments, if the tray is not present or in the correct position, or if there is more than one tray, the next array (805) will not be moved to the tray drawer (920) by the pusher arm system (820).

In some embodiments, the tray drawer system (900) further comprises a safety sensor (970) at the back of the tray drawer (920) that functions to sense if someone reaches past the tray drawer into the order buffer area where the robotic arm system (710) places bottles. The safety sensor (970) can pause movement of the robotic arm system (710) to prevent injury.

The plurality of sensors help keep personnel safe while they collect a completed order, help determine if another array can be moved to the tray drawer via the pusher arm system (820), and to regulate filling of subsequent orders if/when an order is retrieved or remains, and/or an order is moved from the order buffer system (800) to the tray drawer (900), etc.

When an order is ready, healthcare professionals are notified. For example, the system (100) will send a message to the healthcare professional who ordered the order (e.g., via a local application running on their workstation) that the order is complete. In some embodiments, the order status light (182b) is activated when an order is ready to be picked up in the tray drawer (920).

When the healthcare professional is at the tray drawer (920), he/she can, for example: move the door (916) to the open position; grip the tray (910) and slide it out of the tray drawer (920); either empty the order from the tray (910) and place it back in the tray drawer (920) or place another empty tray (910) in the tray drawer (920); and move the door (916) to the closed position.

### Control Center and Software Systems

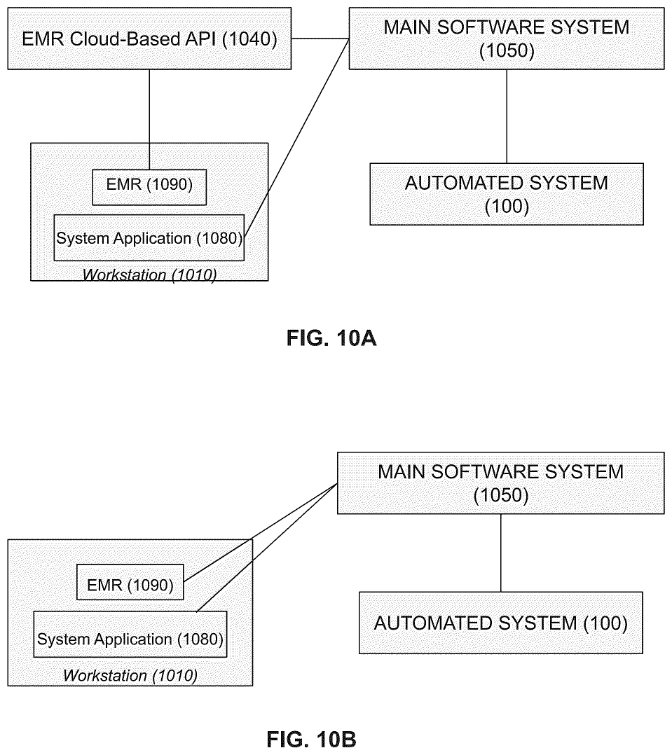

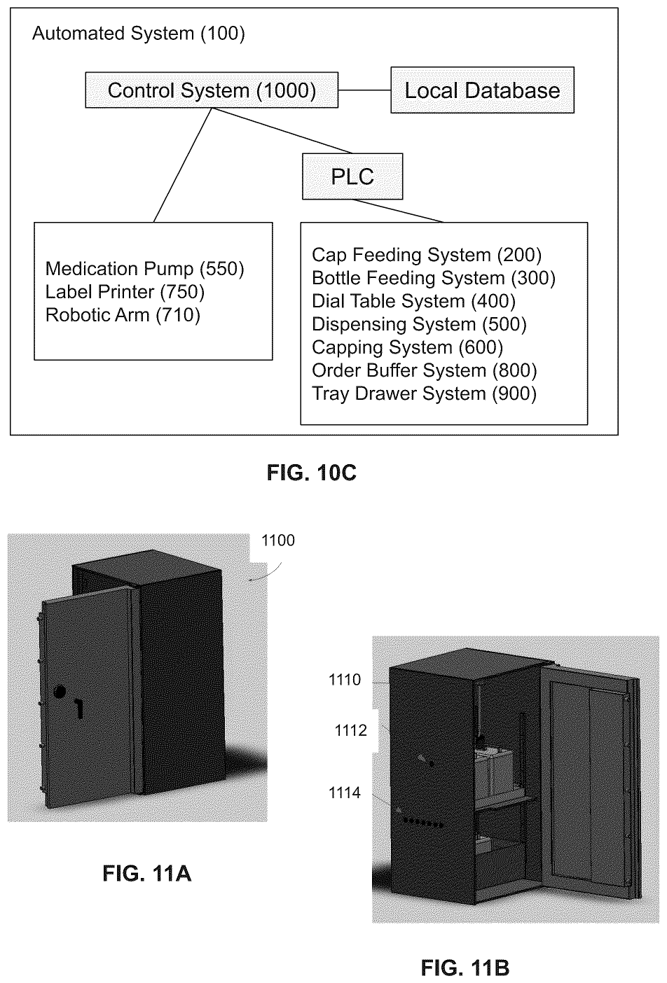

The control system is the system that instructs (and operatively interacts with) at least a portion of the components of the automated system (100) in order to prepare and assemble an array or collection of doses. FIG. 10A and FIG. 10B provide schematic views of communications between the control center or control system (1000) of the automated system (100), the main software system (1050), and the electronic medical record (EMR) system (1090). Also shown are the EMR (1090) and a system application (1080) on the healthcare professional's workstation (1010). In some embodiments, the main software system (1050) is a cloud-based system. In some embodiments, the main software system (1050) runs on a local server. In some embodiments, the EMR (1090) is operatively connected to the main software system (1050). In some embodiments, the EMR (1090) is operatively connected to the main software system via an EMR cloud-based software system (application programming interface (API) (1040). The main software system (1050) may also be referred to as the main application programming interface (API).

FIG. 10C provides a schematic view of the control system (1000) and its relationship to the many systems of the automated system (100) described herein.

As an example, the healthcare professional's workstation (1010) may feature an electronic medical record system (1090). For integration of the automated system (100) of the present invention, the workstation (1010) may also feature a system application (1080), which can provide information about orders submitted by the EMR (1090) at that workstation (1010). For example, the system application (1080) may provide information such as but not limited to a patient-associated ID number, an order number, dispensing status, and a dose count, for example.

The EMR (1090) may be operatively connected to the clinic's software system (1040), e.g., a clinic cloud, and said clinic's software system (1040) may be operatively connected to the main software system (1050), e.g., a cloud based or local server-based system. The main software system (1050) is operatively connected to the automated system (100), e.g., to the control center (1000) of the automated system (100). The main software system (1050) may also be operatively connected to the system application (1080) on the healthcare professional's workstation (1010). The main software system (1050) can buffer multiple orders in order to organize and maintain dose organization and order fulfillment.

As an example, a healthcare professional may order a dose through the EMR (1090), which communicates the order to the clinic software system (1040). Prior to the order being sent to the main software system (1050), the patient information is replaced with a unique patient ID for privacy protection. Thus, the order number is associated with a patient ID rather than a patient name or other data. In some embodiments, the main software system (1050) receives a patient ID in the form of a pdf from the EMR (1090) and/or clinic software system (1040), and the pdf becomes associated with the patient ID. Thus, the main software system (1050) does not handle patient data. The main software system (1050) receives the order and sends the order to the control system (1000) of the automated system (100) for fulfillment. The control system (1000) of the automated system (100) may send status information to the main software system (1050) where it is then transmitted to the system application (1080) on the workstation (1010). In some embodiments, e.g., when an order is complete, information from the control system (1000) is sent to the main software system (1050) whereupon it is then transferred to the clinic software system (1040) and further to the EMR (1090).

As shown in FIG. 10C, the control system (1000) is operatively connected (directly and/or indirectly) to the systems in the automated system (100) such as but not limited to one or a combination of components associated with: the cap feeding system (200), bottle feeding system (300), dial table system (400), dispensing system (500), capping system (600), labeling system (700), order buffer system (800), and the tray drawer system (900). The control system (1000) is operatively connected to the medication pump (550) and the label printer (750). The control system is operatively connected to the robotic arm system (710) of the labeling system (700). In some embodiments, the control system (1000) is operatively connected to a local database.

As previously discussed, the control system is operatively connected to the appropriate individual components of the system, e.g., dispensing system, labeling system, etc.

### Remote Functionalities

As previously discussed, the present invention also provides tamper evident labels for labeling the medication bottles in a way that would make tampering evident and detectable. In some embodiments, the labels are configured to make tampering evident and detectable via remote visualization, e.g., a video camera with remote feed capability, wherein a healthcare professional could observe the patient and medication bottles via the video feed and conduct a telemedicine appointment.

With the ability to detect tampering of a medication bottle remotely, patients would not need to appear in person for medication checks, relieving a burden on the patient. Furthermore, a fast and reliable way to detect medication tampering will help reduce the burden on the healthcare professionals who must check patients' medication, which is often done via time consuming methods such as a dip test or an odor test.

The configuration of the label or the feature of the label that allows for detection or visualization of tampering may include but is not limited to a barcode, a design, a physical feature such as a perforation, an indicator such as a color, etc.

The present invention also provides for remote appointments, which may include the induction time frame wherein a patient must attend a particular series of appointments. A healthcare professional can observe a patient take a dose or perform other actions during the remote appointment.

The present invention also features remote assessment of a patient's status, e.g., cognitive status, brain health, sobriety, etc. For example, the present invention includes methods of assessing a patient's status (e.g., cognitive status, sobriety, etc.) using a system that measures eye movements (e.g., via eye tracking) of a patient for a period of time. As a non-limiting example, systems such as virtual reality (VR) systems or platforms may be employed for measuring eye movements of a patient and processing the data to assess the patient's sobriety. Eye tracking is well known to one of ordinary skill in the art. As an example, eye tracking may be performed by an eye tracking system continuously measuring the distance between the pupil center and the reflection of the cornea of an infrared light emitted by the eye tracking system. The eye tracking system may utilize vision algorithms to determine parameters such as but not limited to where a person's gaze is directed, whether or not the patient is sober, etc.

### Power and Computer-Executable Operations

The system of the present invention features a power source or a plurality of power sources including but not limited to outlet power sources and/or batteries. The system of the present invention may feature a plug for connecting to an outlet power source.

Instructions that cause at least one processing circuit to perform one or more operations are “computer-executable.” Within the scope of the present invention, “computer-readable memory,” “memory component,” and the like comprises two distinctly different kinds of computer-readable media: physical storage media that stores computer-executable instructions and transmission media that carries computer-executable instructions. Physical storage media includes RAM and other volatile types of memory; ROM, EEPROM and other non-volatile types of memory; CD-ROM, CD-RW, DVD-ROM, DVD-RW, and other optical disk storage; magnetic disk storage or other magnetic storage devices; and any other tangible medium that can store computer-executable instructions that can be accessed and processed by at least one processing circuit. Transmission media can include signals carrying computer-executable instructions over a network to be received by a general-purpose or special-purpose computer. Thus, it is emphasized that (by disclosure or recitation of the exemplary term “non-transitory”) embodiments of the present invention expressly exclude signals carrying computer-executable instructions. However, it should be understood that once a signal carrying computer-executable instructions is received by a computer, the type of computer-readable storage media transforms automatically from transmission media to physical storage media. This transformation may even occur early on in intermediate memory such as (by way of example and not limitation) a buffer in the RAM of a network interface card, regardless of whether the buffer's content is later transferred to less volatile RAM in the computer.

### Safe

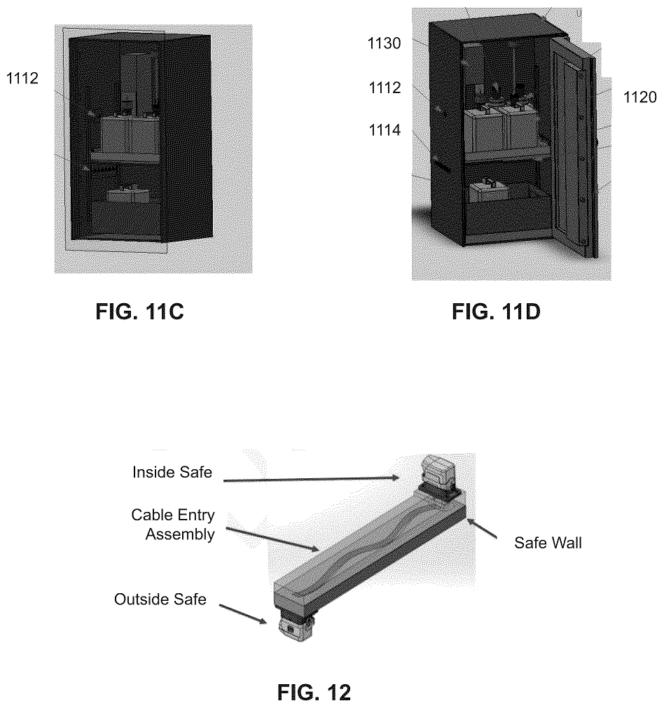

Referring now to FIG. 11A, FIG. 11B, FIG. 11C, and FIG. 11D, the present invention features a safe (1100) for remote automated dispensation of controlled substance dosages. In some embodiments, the safe (1100) may comprise a safe housing (1110). The safe housing (1110) may comprise a dispense port (1112) disposed through the safe housing (1110). The safe housing (1110) may further comprise a plurality of power and control lead ports (1114) disposed through the safe housing (1110).

The safe (1100) may further comprise a liquid dosage container or a number of containers (1120) containing a plurality of controlled substance dosages and one or more pumping and/or dispense mechanisms (1130) operatively coupled to a dosage container (1120) and the dispense port (1112), or the safe (1100) may be configured to integrate one or more pumping mechanisms. The pumping and/or dispense mechanisms (1130) may be powered and controlled by a plurality of wires directed through the plurality of power and control lead ports (1114).

The safe (1100) may further comprise an actuation component (1140) operatively coupled to the one or more dispense mechanisms and/or one or more pumping mechanisms (1130). Actuating the computer actuation component may cause the one or more dispense mechanisms and/or one or more pumping or dispense mechanisms (1130) to draw a controlled substance dose from the dosage container or containers (1120), and direct the controlled substance dose to the bottling and/or packaging mechanism before it is progressed to the dispense port (1112) to be retrieved by a user.

Referring now to FIG. 11D, in certain embodiments, an interior of the safe may comprise a suction connection operatively connecting the dosage container (1120) to the one or more pump mechanisms (1130). The suction connection may comprise a switch that is actuated by the computer actuation component (1140), and the switch may control the suction of the suction connection. The interior of the safe (1100) may further comprise a pump cleaning solution container operatively connected to the one or more pump mechanisms (1130). The one or more pump mechanisms (1130), after dispensing a controlled substance dosage, may pump cleaning solution from the pump cleaning solution container through the suction connection and the one or more pump mechanisms (1130) to clean out residue medication from dispensing the last dose. In some embodiments, the dosage container (1120) may be placed in a drip pan to catch any spills. The interior of the safe may further comprise a waste collection container for collecting the pumped cleaning solution and any residual medication.

The present invention is not limited to the aforementioned configurations of the safe.

In some embodiments, a controlled substance dosage comprises methadone, buprenorphine, buprenorphine/nalaxone, or a combination thereof in liquid, tablet, or sublingual strip form. The present invention is not limited to the aforementioned examples of medications or formulations.

The present invention also features a method for remote automated dispensation of controlled substance dosages featuring the use of a safe (1100).

### Treatment Booth System

The present invention also features remote treatment booths (booth systems) for providing a remote interface between healthcare professionals and patients, which may be used for securely delivering opioid treatment doses. The booth systems help allow opioid recovery patients to receive communication and treatment from a healthcare professional efficiently without needing to travel to the home OTP for every encounter. The booth system can receive orders locally (e.g., for in clinic) or remotely (e.g., when the booth system is remote). For example, the booth system can receive orders remotely from a healthcare provider/OTP (or other appropriate remote provider) when the booth system is remote.

The booth system may be split or separated into two (or at least two) portions. For example, the booth system may comprise a patient space (e.g., user interface chamber (1400)) and an automated system, e.g., automated system (110), automated system (1500) which features a safe etc. As previously discussed, the automated systems (110, 1500) may be used independently or separately from the booth system to just support dose assembly. For example, the automated systems may be used independently behind the counters in clinics, pharmacies, FQHCs, IHSs, etc., e.g., independently from the patient space (e.g., user interface chamber (1400)). In some embodiments, the automated systems (110, 1500) work in conjunction with the patient space (e.g., user interface chamber (1400)) that might be located in a patient accessible area like a pharmacy retail floor.

As a non-limiting example, a patient may have a visit in the patient space (user interface chamber (1400)), wherein the healthcare provider will send approval and the order to the automated system (110, 1500) behind the counter. The patient may then go to the counter to pick up his/her doses that were prepared by the automated system (110, 1500) behind the counter. This automated system (110, 1500) behind the counter can be configured to receive both local and remote orders when separated.

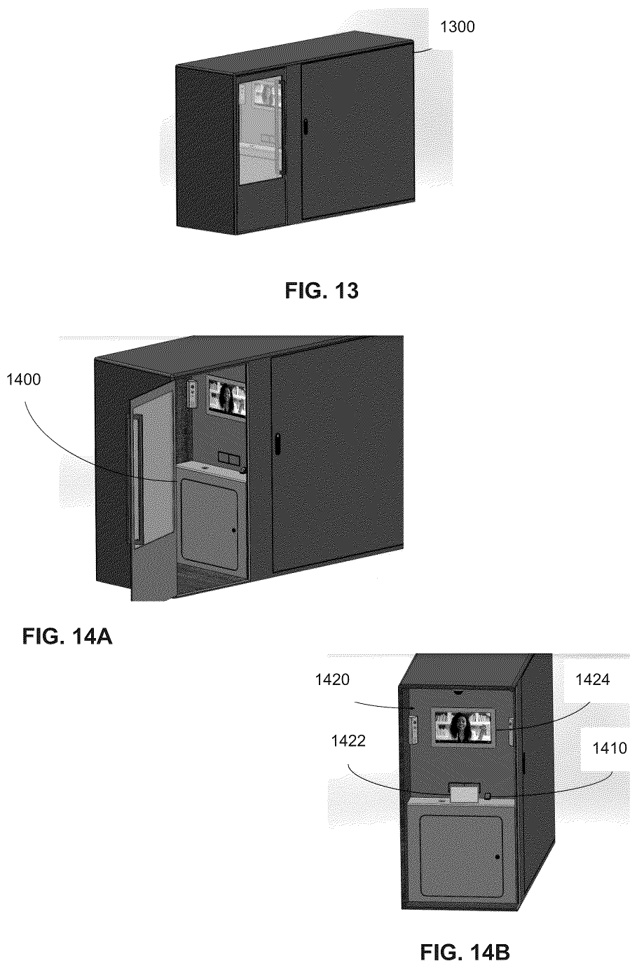

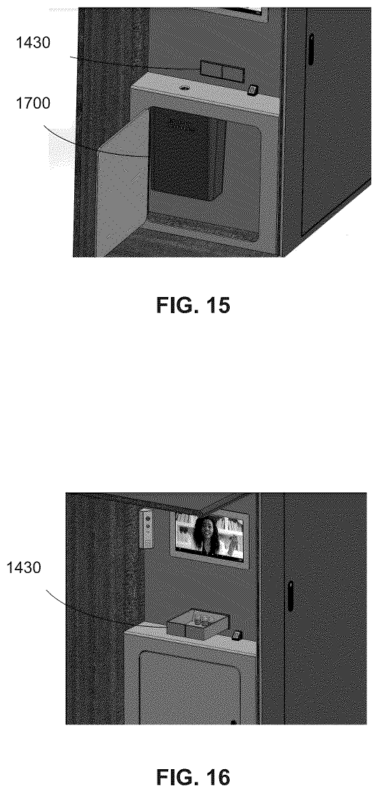

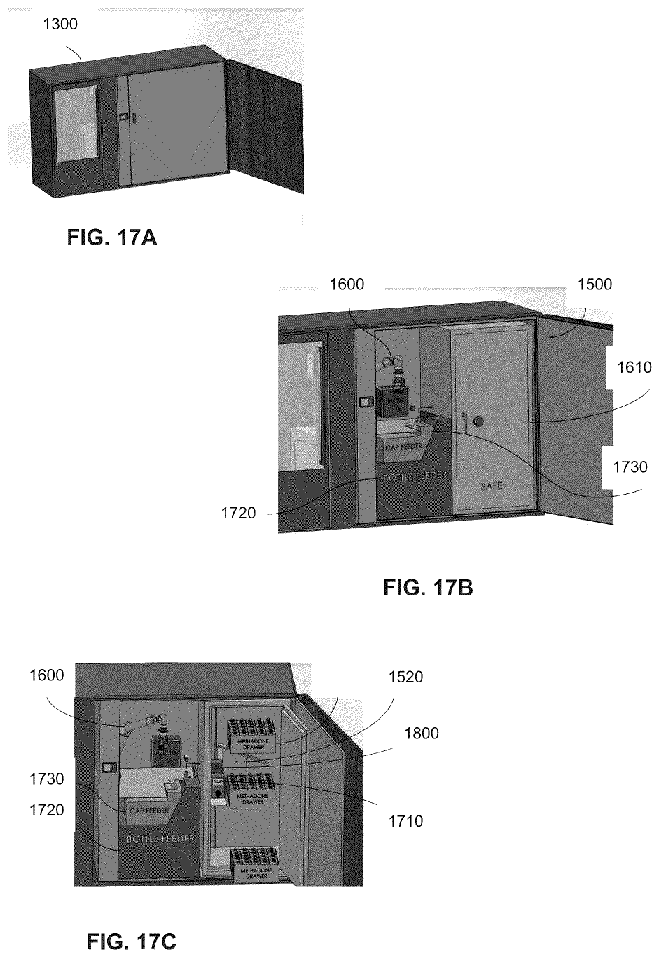

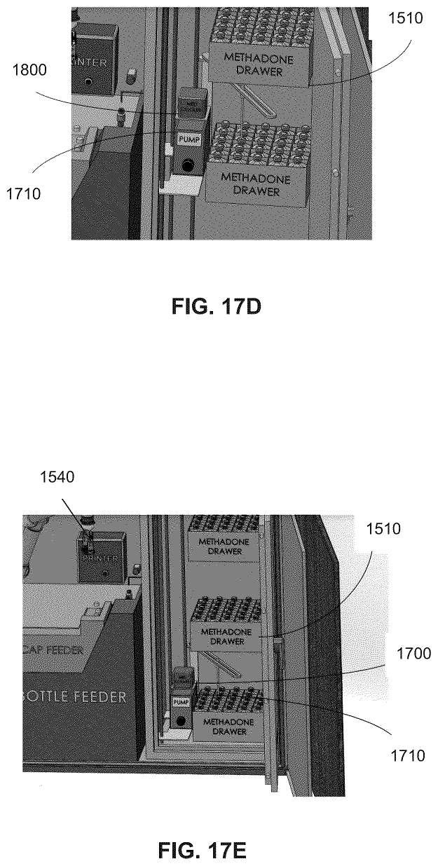

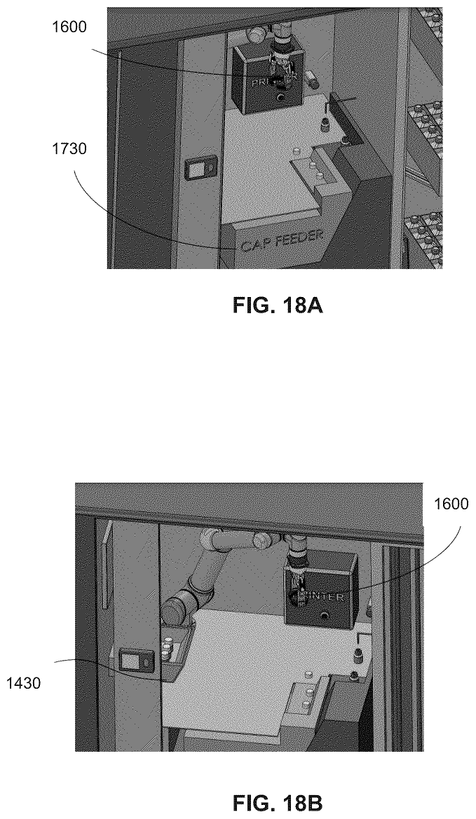

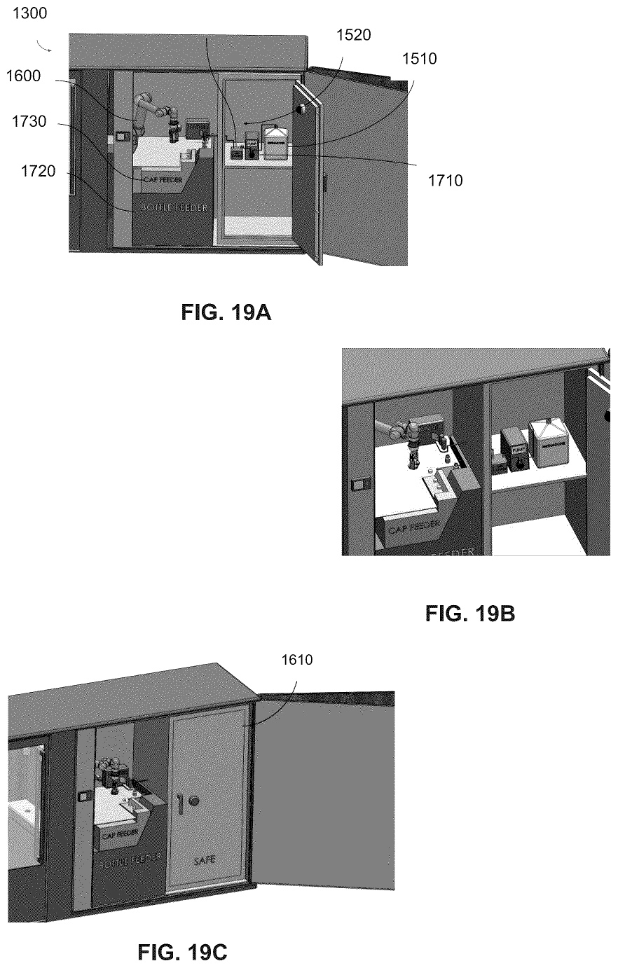

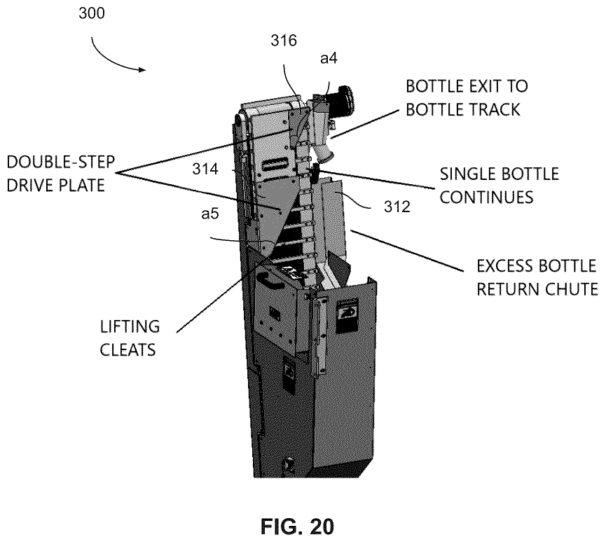

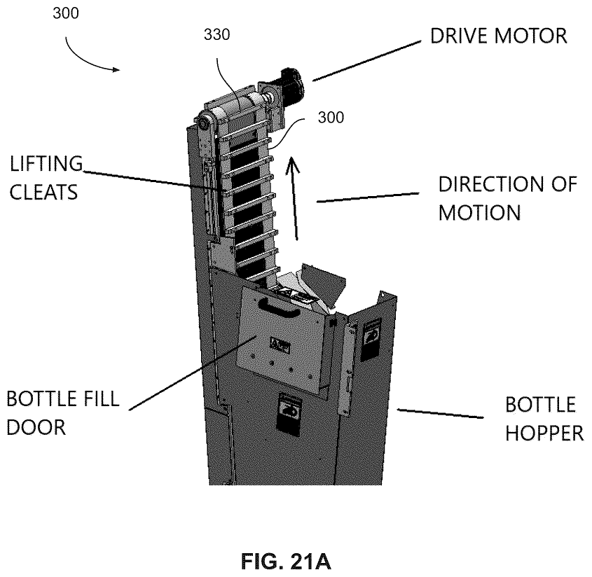

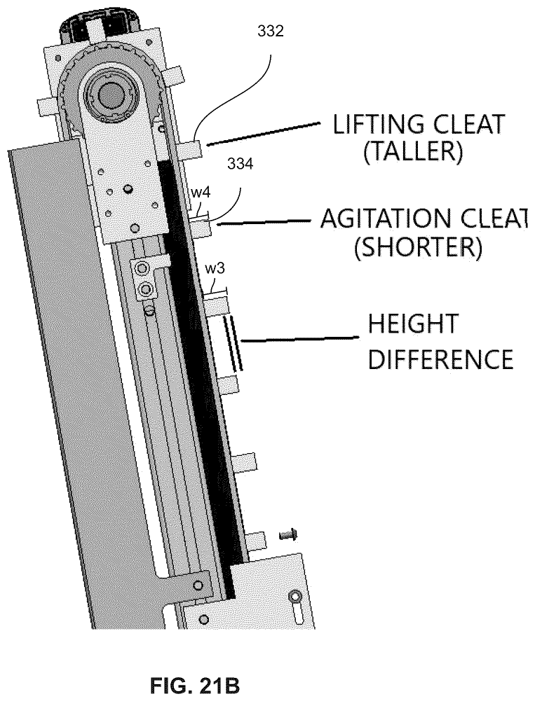

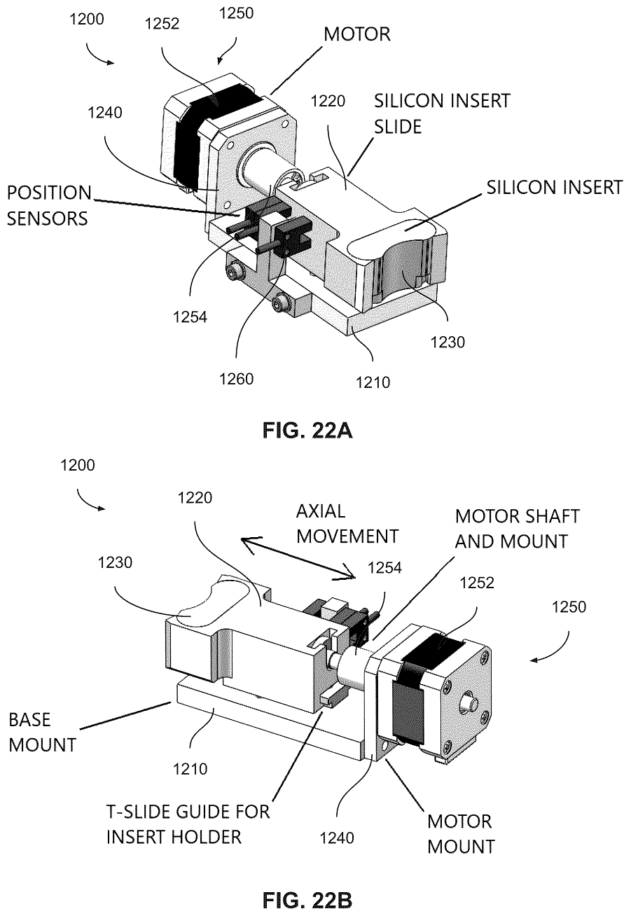

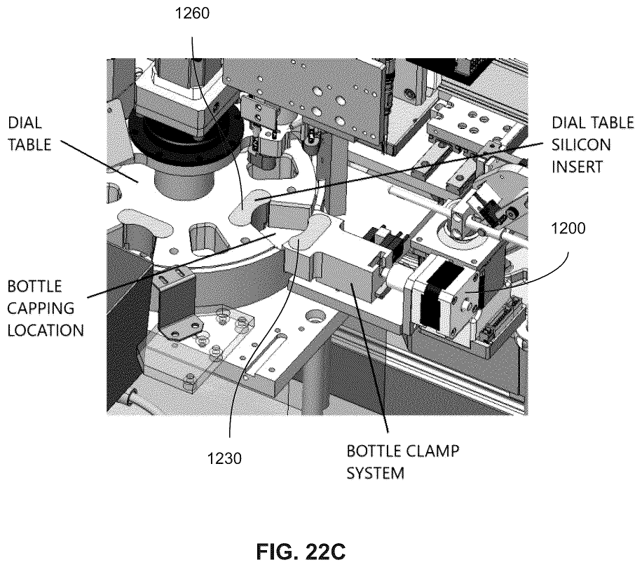

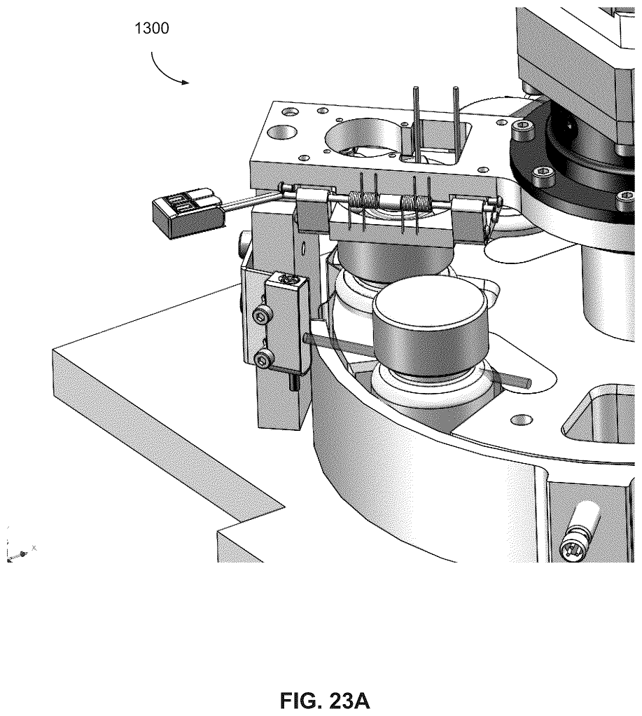

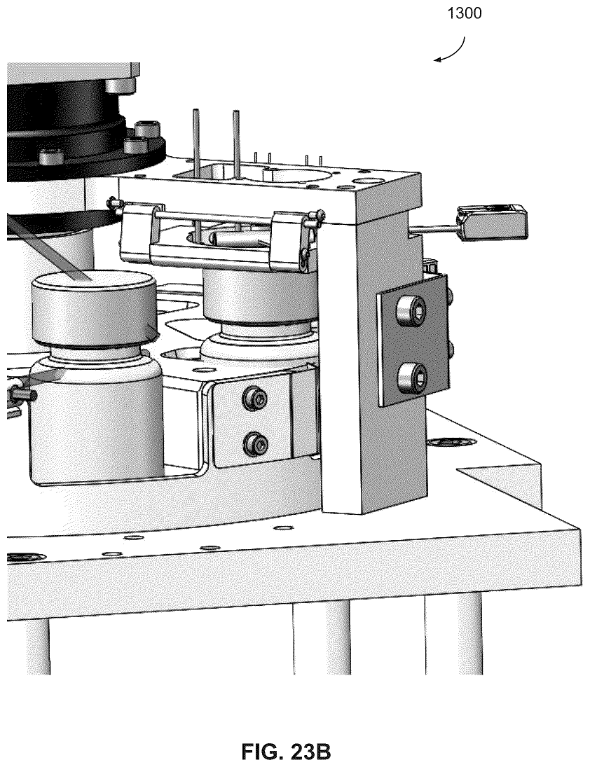

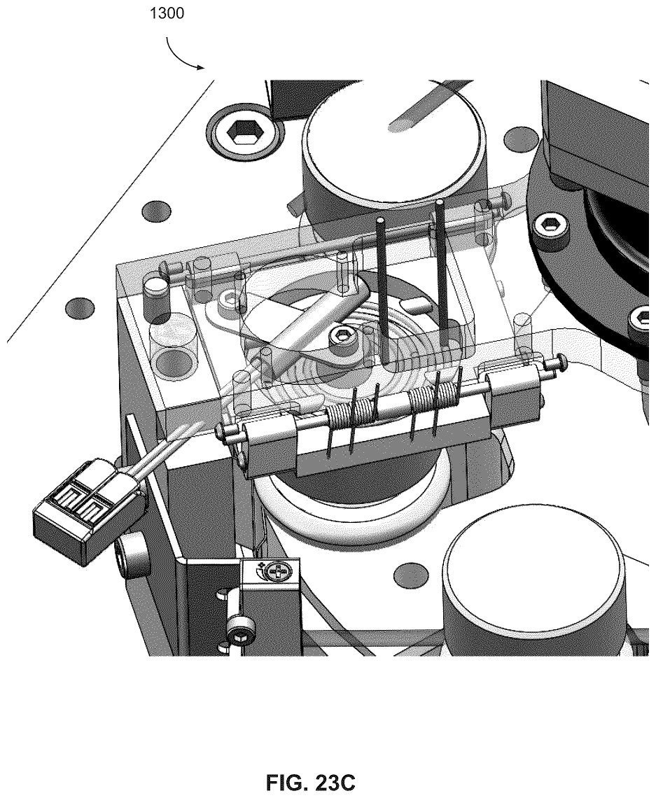

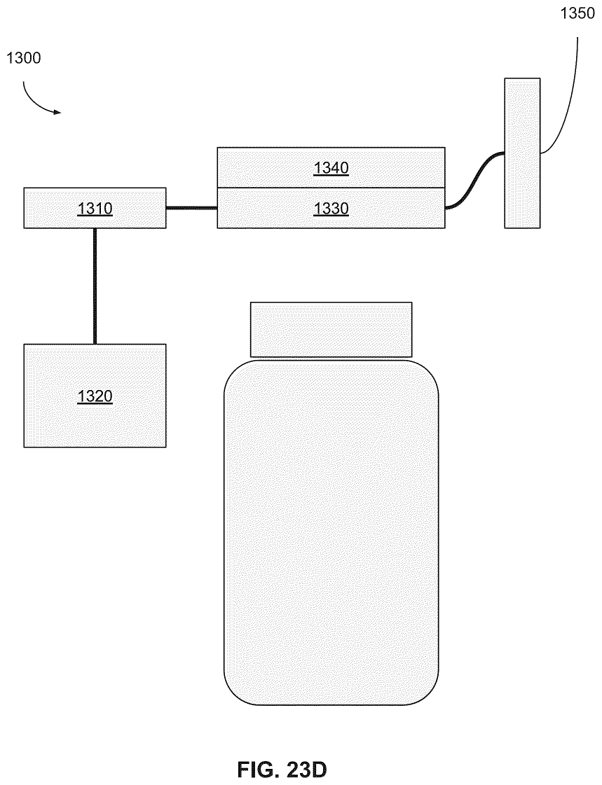

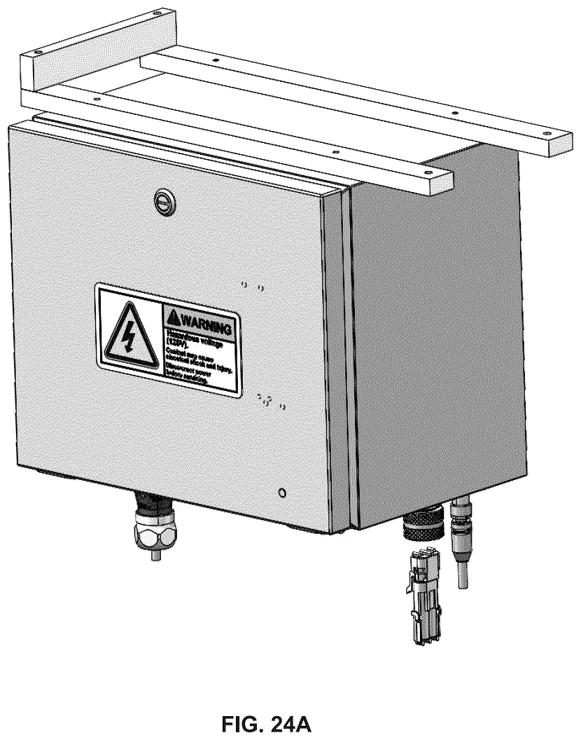

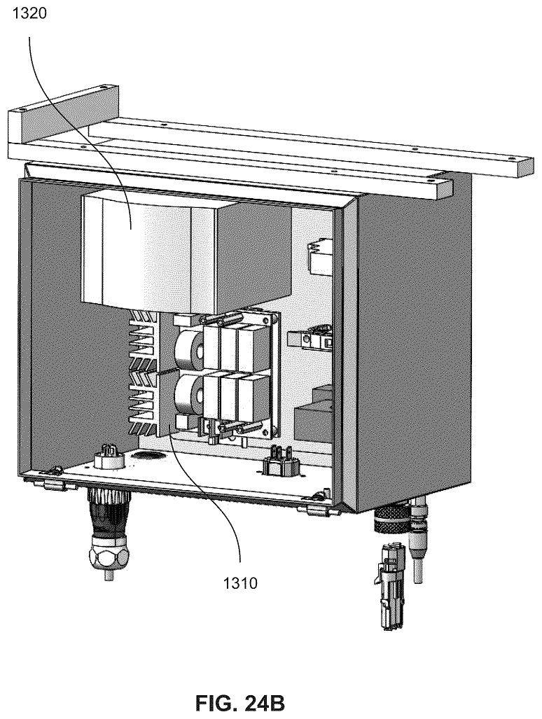

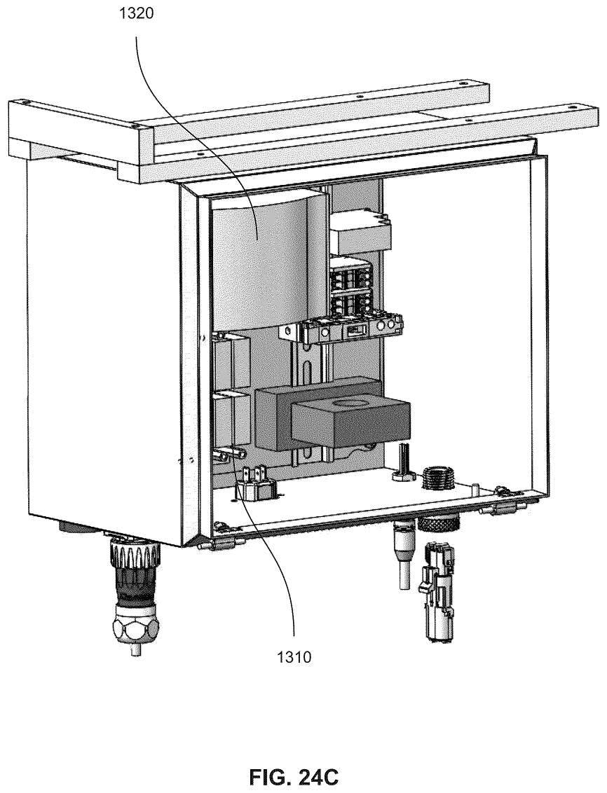

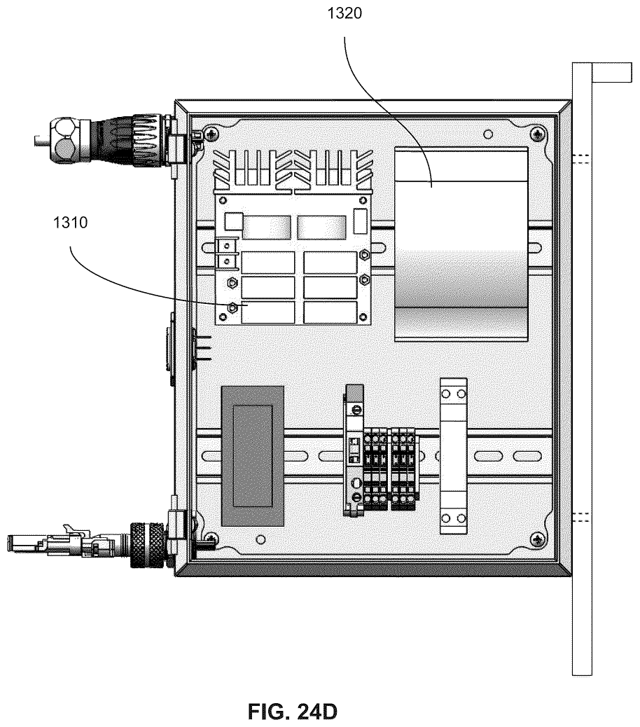

Referring now to FIG. 13, FIG. 14A, FIG. 14B, FIG. 15, FIG. 16, FIG. 17A, FIG. 17B, FIG. 17C, FIG. 17D, FIG. 17E, FIG. 18A, FIG. 18B, FIG. 19A, FIG. 19B, and FIG. 19C, the present invention features an opioid treatment booth system (1300). In some embodiments, the opioid treatment booth system (1300) may comprise a user interface chamber (1400) for housing a user and allowing the user to interface with a healthcare professional. The user interface chamber (1400) may comprise a biometric input component (1410) capable of accepting a biometric input from the user as a means of authorization. The biometric input of the user may be registered to the opioid treatment booth (1300), thus allowing the user to be assigned to a specific opioid treatment booth (1300) location or to utilize treatment booths within a network. In some embodiments, the healthcare professional may be capable of changing the opioid treatment booth location that the user is assigned to, thus changing the opioid treatment booth where their biometric input is accepted. Authorizing the biometric input component (1410) may be capable of actuating an interface system (1420) and a treatment delivery component (1430). In some embodiments, the biometric input component (1410) may further accept a user ID number in addition to the biometric input as the means of authorization. The biometric input may comprise a fingerprint, a facial scan, a retinal scan, a palm-vein scan, or a combination thereof. The biometric input component (1410) may be operatively coupled to a computing device comprising a processor capable of executing computer-readable instructions and a memory component comprising a plurality of computer-readable instructions for accepting input from the biometric input component (1410), checking if the input matches stored biometric data, and, if the input matches the stored biometric data, authorizing the interface system (1420) and the treatment delivery component (1430). The treatment delivery component (1430) may additionally require authorization from the healthcare professional. The healthcare professional may be a nurse, a doctor, a pharmacist, or any other licensed official authorized to monitor the user's treatment.

The user interface chamber (1400) may further comprise an input component (1422) operatively coupled to the biometric input component (1410) capable of allowing the user to communicate with the healthcare professional, and a display component (1424) capable of allowing the healthcare professional to communicate with the user. In some embodiments, the input component (1422) may comprise a touch screen, a keyboard, a microphone, a standard camera, a thermal image camera, or a combination thereof. The thermal image camera, in particular, may allow the healthcare professional to see the cooled medication if the user diverts the medication into their pocket or if they try to divert treatment in any way. The touch screen, in particular, may allow the user to answer questions regardless of language spoken or literacy. In some embodiments, the display component (1424) may comprise a screen, a speaker, or a combination thereof. The display component (1424) may allow the user to view a number of days in treatment. The interface system (1420) may require the user to answer questions displayed through the display component (1424) through the input component (1422). The input component (1422) records the user for future review or may allow the healthcare professional to snap images of the treatment dose.

The interface system (1420) may further comprise a communication component for allowing the opioid treatment booth (1300) to communicate with the healthcare professional in a remote location. The communication component may transmit and receive data over a wired or a wireless Internet connection. The interface system (1420) may further comprise a computing device operatively connected to the input component (1422) and the display component (1424). The computing device may comprise a processor capable of executing computer-readable instructions and a memory component comprising a plurality of computer-readable instructions for accepting authorization from the biometric input component (1410), transmitting, by the communication component, data from the input component (1422), and receiving, by the communication component, data from the remote location to be displayed on the display component (1424). The communication component may allow for secure and/or encrypted communication. The biometric authorization allows for the user to be identified securely without the need for the healthcare professional to check their ID.

The user interface chamber (1400) may further comprise the treatment exchange component (1430) capable of accepting a treatment dose from a treatment containment chamber (1500). The treatment exchange component (1430) may comprise a drawer. The treatment exchange component (1430) may be locked into a wall of the user interface chamber (1400) until authorization has been received from the biometric input component (1410). The treatment exchange component (1430) may additionally have a standard lock capable of being opened by a key owned by the healthcare professional.

The opioid treatment booth (1300) may further comprise the treatment containment chamber (1500). The treatment containment chamber (1500) may comprise a treatment storage container (1510) which may comprise a plurality of treatment doses in a liquid or solid dosage form. The plurality of treatment doses may comprise methadone, buprenorphine, buprenorphine/naloxone, or a combination thereof. The treatment storage container (1510) may be contained in a safe (1610). The treatment containment chamber (1500) may further comprise a treatment delivery component (1520) operatively coupled to the treatment exchange component (1430) capable of delivering one or more treatment doses to the treatment exchange component (1430). The one or more treatment doses may be labeled as either an observed dose to be taken by the user in the user interface chamber (1400), or a take-home dose that does not need to be observed as it is taken. The one or more liquid treatment doses may be extracted by a medication pump (1710) of the treatment delivery component (1520) into a container.

In some embodiments, the user interface chamber (1400) may further comprise a non-retrievable disposal component (1700) for the user to dispose of a portion of the treatment dose after taking it while in communication with the healthcare professional. The user may additionally dispose of a container that once contained the treatment dose in the disposal component (1700). The disposal component (1700) may comprise a trash can.

The healthcare professional, while connecting to the opioid treatment booth (1300), may be able to view the user's ID number, location, wait time, and notifications. Additionally, the healthcare professional may be able to register a flag to the user if the user attempts to divert treatment. This may be an adjustment of medicine amount or medicine used in the treatment dose. This may be possible through data received by the communication component of the computing device of the interface system (1420). The healthcare professional may be able to view a total number of treatment doses contained in the treatment storage container (1510).

In some embodiments, the opioid treatment further may comprise a robotic arm (1600) disposed in the treatment containment chamber (1500). The robotic arm (1600) may be connected to a computing component. The computing component may comprise a processor capable of executing computer-readable instructions and a memory component comprising computer-readable instructions for moving the treatment dose from the treatment delivery component (1520) to the treatment exchange component (1430), removing an unused treatment dose from the treatment delivery component (1520), placing the unused treatment dose into a quarantine zone in the treatment containment chamber (1500), and pouring the unused treatment dose into the disposal component (1700) and adjusting the total number of treatment doses contained in the treatment storage container (1510) accordingly. The healthcare professional may be capable of actuating the robotic arm (1600) by data received by the communication component.

In some embodiments, the treatment storage container (1510) may comprise a cooling component (1800) capable of refrigerating the treatment dose. This may only be actuated on treatment doses labeled as an observed dose. The treatment delivery component (1520) may deliver the treatment dose by dispensing, by a pump (1710), the treatment dose into a container to be transferred to the user through the treatment exchange component (1430). In some embodiments, the container may comprise a bottle, and the treatment delivery component (1520) may comprise a bottle feeder (1720) and a cap feeder (1730) to allow the treatment dose to be dispensed into the bottle and for the bottle to be sealed with a cap. The treatment delivery component (1520) may be controlled by a computing component comprising a processor capable of executing computer-readable instructions and a memory component comprising computer-readable instructions for accepting authorization from the biometric input component (1410), actuating the pump (1710) to dispense a treatment dose from the treatment storage container (1510), optionally cooling the treatment dose by the cooler (1800), positioning, by the bottle feeder (1720), a bottle to receive the treatment dose, and sealing, by the cap feeder (1730), the bottle with a cap. In some embodiments, the healthcare professional may be able to access the treatment containment chamber (1500) for refills. In some embodiments, the opioid treatment booth (1300) may further comprise a printer (1540), for labeling the bottles with patient specific information in the case of a take-home dose. In some embodiments, observed doses may not be labeled.

In some embodiments, the treatment storage container (1510) may comprise a plurality of individual treatment dose containers. Actuating the treatment delivery component (1520) may cause the pump (1710) to extract the treatment dose from an individual treatment dose container. Subsequent actuations of the treatment delivery component (1520) may cause the pump (1710) to extract from a different individual treatment dose container (e.g. adjacent to the previous container, on a different level from the previous container, etc.). In other embodiments, the treatment storage container (1510) may comprise a single vat containing the plurality of treatment doses. Actuation of the treatment delivery component (1520) may cause the pump (1710) to extract the treatment dose from the single vat.

The present invention features a medication pump accessory system for automated assembling of an array of doses of a medication. In some embodiments, the system may comprise a control center (190) configured to receive an order for an array of doses of the medication directly or indirectly from an electronic medical record (EMR) system. The system may further comprise a dial table system (400) comprising at least four slots therein, configured to temporarily hold a medication bottle. Each of the at least four slots may occupy either a loading position, a dispensing position, a capping position, or an unloading position, the dial table system (400) further configured to rotate to move slots from one position to another. The system may further comprise a dispensing system (500) configured to aliquote a unit of medication from a reservoir through a dispenser into the medication bottle in the slot occupying the dispensing position of the dial table system (400), the dispensing system (500) comprises a means of connecting a medication pump. The system may further comprise a capping system (600) configured to securely attach a cap to the medication bottle in the capping position of the dial table system (400).

In some embodiments, the system may further comprise a bottle feeding system (300) configured to obtain a medication bottle from a bottle storage hopper and directing the medication bottle to a slot occupying the loading position of the dial table system (400). In some embodiments, the bottle feeding system (300) may comprise a belt feeder (310) comprising a belt (320), configured to rotate around an elongated plate or elongated axis formed by a drive gear (362) operatively connected to a drive motor (360) and a belt wheel (3242). The bottle feeding system (300) may further comprise a plurality of cleats (330) disposed on the belt (320) spaced a distance d2 apart. A lower end of the belt feeder (310) may be configured to engage bottles housed in a bottle storage hopper such that as the belt (320) rotates the cleats (330) rise and capture one or more bottles thereon as the cleats (330) rise towards a top end of the belt feeder (310). The belt feeder (310) may be oriented at an angle a1 with respect to horizontal. The bottle feeding system (300) may further comprise a bottle delivery guide (380) configured to deliver bottles (305) from the belt feeder (310) to the slot occupying the loading position of the dial table system (400).

In some embodiments, the system may further comprise an excess bottle return chute (312) disposed adjacent to the belt feeder (310), configured to deliver bottles to the bottle storage hopper. The system may further comprise a first guide plate (314) disposed at or near a bottom end of the belt feeder (310), configured to cause movement of bottles in an improper configuration on the belt (320) to the excess bottle return chute (312). The system may further comprise a second guide plate (316) disposed at or near the top end of the belt feeder (310), configured to cause movement of the bottles (305) to the bottle delivery guide (380).

In some embodiments, the first guide plate (314) may comprise an angle a4 with respect to vertical. The second guide plate (316) may comprise an angle a3 with respect to vertical. In some embodiments, the angle a4 may determine a speed at which the bottles in the improper configuration are moved to the excess bottle return chute (312). In some embodiments, the angle a5 may determine a speed at which the bottles (305) are moved to the bottle delivery guide (380). In some embodiments, only one bottle per cleat can pass the second guide plate (316) and reach an exit point. In some embodiments, the first guide plate (314) and the second guide plate (316) may be integrated into a double-guide plate.

In some embodiments, this double-step drive plate design may allow for the cleats to lift multiple bottles per cleat, but the first (lower) drive plate pushes all but one bottle off. Any additional bottles are returned via the excess bottle return chute, leaving only one bottle to progress upwards toward the second (higher) drive plate. At this drive plate, the bottle is then pushed off the cleat where it simply free-falls into the bottle exit to the bottle track, which feeds down into the rest of the automation system. In some embodiments, the first drive plate, the second drive plate, or a combination thereof may be configured to push off the bottle in an improper configuration. In some embodiments, the improper configuration may comprise a vertical configuration, a backwards configuration (e.g. the cap-side facing towards the bottle delivery guide, etc.), multiple bottles on a cleat, or a combination thereof.

In some embodiments, a larger angle a4 and/or a5 may cause the bottles to be pushed off of the belt sooner. Inversely, a smaller angle a4 and/or a5 may cause the bottles to be pushed off of the belt later. In some embodiments, angle a4 may be 30 to 60 degrees. In some embodiments, angle a5 may be 30 to 60 degrees. In some embodiments, angle a4 may be different from angle a5. In other embodiments, angle a4 may be equal to angle a5. In some embodiments, angle a4, angle a5, or a combination thereof may be determined relative to a spacing between cleats of the plurality of cleats.

In some embodiments, only one bottle may be in range of the second drive plate at a time. In some embodiments, the first drive plate, the second drive plate, or a combination thereof may be fixed to the bottle feeding system. In some embodiments, the first drive plate, the second drive plate, or a combination thereof may be adjustable in position so as to change a speed at which the bottle is pushed from the belt. In some embodiments, moving the first drive plate closer to the excess bottle return chute may cause bottles to be pushed into the excess bottle return chute sooner. Inversely, moving the first drive plate further from the excess bottle return chute may cause the bottles to be pushed into the excess bottle return chute later. In some embodiments, moving the second drive plate closer to the bottle delivery guide may cause the bottles to be pushed into the bottle delivery guide sooner. Inversely, moving the second drive plate further from the bottle delivery guide may cause the bottles to be pushed into the bottle delivery guide later.

In some embodiments, the first drive plate, the second drive plate, or a combination thereof may be slidably coupled to the belt such that the first drive plate, the second drive plate, or the combination thereof can be easily slid to any desired distance relative to the to the excess bottle return chute and the bottle delivery guide. In some embodiments, the first drive plate, the second drive plate, or a combination thereof may be bolted in place. In some embodiments, the bottle feeding system may comprise a plurality of drive plate holes defining a plurality of configurations for the first drive plate, the second drive plate, or a combination thereof to be bolted into (e.g. closer to or further from the excess bottle return chute and/or the bottle delivery guide). In some embodiments, the plurality of drive plate holes may be threaded and configured to accept threaded screws. In some embodiments, the plurality of drive plate holes may be non-threaded and configured to accept non-threaded pegs.

In some embodiments, the first drive plate and the second drive plate may be separate components such that each drive plate can be adjusted individually. In some embodiments, the first drive plate and the second drive plate may be linked by a linking plate such that the first drive plate and the second drive plate are fixed in relative position to each other. In some embodiments, the first drive plate and the second drive plate may be integrated into a single plate component.

This cleat configuration allows for the correct timing to allow bottle singulation. Every other cleat is a shorter agitation cleat (non-suitable). It is shorter in height or distance from the yellow drive belts, this distance is such that a bottle will not physically be carried up on it while laying horizontally—it falls off because the bottle's center of gravity is out past the edge of the cleat. This cleat is simply used to agitate the bottles in the hopper. It is also used to book bottles from standing up on the lifting cleat (suitable). Every other cleat is a taller lifting cleat (suitable), which is a larger distance from the yellow drive belts. This cleat is the correct height such that a bottle will be carried up while laying horizontally. It stays on because the bottle's center of gravity is inside the width of the cleat.

In some embodiments, the plurality of cleats (330) may comprise a first set of cleats (332) and a second set of cleats (334). In some embodiments, each cleat of the first set of cleats (332) may comprise a width w3 extending from the belt (320). In some embodiments, the first set of cleats (332) may be suitable for supporting the bottles (205). In some embodiments, each cleat of the second set of cleats (334) may comprise a width w4 extending from the belt (320). In some embodiments, the second set of cleats (334) may be unsuitable for supporting the bottles (205). In some embodiments, the second set of cleats (334) may be alternately disposed between the first set of cleats (332).

In some embodiments, the width w3 may comprise a width of 15 to 20 mm. In some embodiments, the width w4 may comprise a width of 10 to 15 mm. In some embodiments, a spacing between each pair of cleats of the plurality of cleats may be 50 to 75 mm. In some embodiments, the width w3 may be determined based on a known size of the bottles such that a center of gravity of each bottle is supported by the first set of cleats. In some embodiments, the width w3 may be greater than or equal to a diameter of each bottle. In some embodiments, the width w4 may be determined based on a known size of the bottles such that a center of gravity of each bottle is not supported by the second set of cleats. In some embodiments, the width w4 may be less than a diameter of each bottle. In some embodiments, the second set of cleats may be configured to agitate the bottle in the bottle feed hopper,

In some embodiments, the system may further comprise a clamping system (1200) configured to hold the medication bottle in place in a slot of the dial table system (400). The clamping system (1200) may comprise a base mount (1210) having a front end and a back end. The clamping system (1200) may further comprise a clamping arm (1220) having a front end and a back end, slidably disposed on the base mount (1210). The clamping system (1200) may further comprise an insert (1230) disposed at the front end of the clamping arm (1220), the insert (1230) comprising a concave curved shape matching a curvature of the medication bottle. The clamping system (1200) may further comprise a motor mount (1240) operatively coupled to the back end of the base mount (1210). The clamping system (1200) may further comprise a clamping motor mechanism (1250) comprising a motor (1252) operatively coupled to the motor mount (1240), and a shaft (1254) operatively coupled to the motor (1252) and the back end of the clamping arm (1220). The clamping motor mechanism (1250) may be configured to extend the shaft (1254) such that the clamping arm (1220) applies pressure to the medication bottle, and retract the shaft (1254) such that the clamping arm (1220) does not contact the medication bottle.

The clamping system (1200) may further comprise one or more position sensors (1260) operatively coupled to the base mount (1210) of the clamping system (1200), configured to detect a distance between the insert (1230) and the medication bottle, and stop the motor (1252) when the distance between the insert (1230) and the medication bottle reaches a threshold. In some embodiments, each slot of the at least four slots of the dial table system (400) may comprise a dial table insert (1270) disposed in the slot, the dial table insert (1270) having a concave curved shape matching a curvature of the medication bottle.

This clamping system allows for a more dispersed pressure and surface friction on the bottle without having to apply as much pressure. Prior clamp designs would dimple the bottle slightly, which was found to occasionally cause a labeling issue due to loss of concentricity. The clamping system of the present invention uses a silicon molded insert that interfaces and matches the contour of the bottle. The inner part of the dial table (the four place wheel that rotates) also uses this same silicon molded insert to provide substantial surface contact to eliminate bottle rotation while the cap is screwed on to 1.5-2 n of force.

In some embodiments, the insert may comprise a silicon material. In some embodiments, the insert may comprise any material having about 70 durometer. In some embodiments, the insert may comprise a material configured to grip the bottle. In some embodiments, the dial table insert may comprise a silicon material. In some embodiments, the dial table insert may comprise any material having about 70 durometer. In some embodiments, the dial table insert may comprise a material configured to grip the bottle. In some embodiments, the position sensors may prevent the clamping arm from moving further than 1 mm after initially contacting the bottle. In some embodiments, when the insert and the dial table insert are contacting the bottle, the insert and the dial table insert may encompass around 50% of the circumference of the bottle. In some embodiments, the clamping system may be coupled to the dial table system such that the clamping arm is configured to hold the bottle in place in the loading position. In some embodiments, the clamping system may be coupled to the dial table system such that the clamping arm is configured to hold the bottle in place in the dispensing position. In some embodiments, the clamping system may be coupled to the dial table system such that the clamping arm is configured to hold the bottle in place in the capping position. In some embodiments, the clamping system may be coupled to the dial table system such that the clamping arm is configured to hold the bottle in place in the unloading position.

In some embodiments, the cap may comprise a foil layer coupled to an interior of the cap by a meltable seal. In some embodiments, the system may further comprise a heat sealing system (1300) operatively coupled to the unloading position of the dial table system (400), configured to melt the meltable seal of the cap such that the foil layer is applied to the medication bottle. In some embodiments, the heat sealing system (1300) may comprise a frequency generator (1310), configured to generate a frequency. The heat sealing system (1300) may further comprise a power supply (1320) operatively coupled to the frequency generator (1310), configured to supply power to the frequency generator (1310). The heat sealing system (1300) may further comprise a coil (1330) operatively coupled to the frequency generator (1310), configured to accept the frequency and generate a heat field such that the meltable seal melts such that the foil layer is applied to the medication bottle.

In some embodiments, the heat sealing system (1300) may further comprise a cooling device (1340) operatively coupled to the coil (1330), configured to cool the coil (1330) after the heat field is generated. The heat sealing system (1300) may further comprise a thermal fuse (1350) operatively coupled to the coil, configured to detect a temperature of the coil (1330), and shut down the power supply (1320) if the temperature of the coil (1330) exceeds a temperature threshold. In some embodiments, the capping system (600) may be further configured to overtorque the cap onto the medication bottle.

The heat sealing system allows for the option to use foil caps which are heat sealed to the top of the bottle, just like a typical liquid type of medication. The caps have a foil liner that is induction heated which melts and seals to the top of the bottle after the child-proof cap is screwed on. The cap loosens some after heat sealing because there is a layer in the foil liner that melts and reduces in thickness. This requires the caps to be “over-torqued” to account for this loosening and yet stay within the child-proof nature or challenge of opening. The foil seal is a much more secure seal. Clinics are required to perform “call-backs” on patients to confirm they are not “diverting” medication. This is much easier to do with the alternative cap liners which are a foam “pressure seal”. In this liner, a seal is made with a pressure sensitive/activated adhesive. This adhesive can be lifted-up and reapplied and is very subjective for “call-backs” in comparison to the heat-sealed foil liners. Thus, heat-sealed foil liners may be implemented in the bottles of the present invention.

In some embodiments, the heat sealing system may be positioned relative to the dial table system such that the bottle is directly in the center of the coil when the bottle is in the unloading position. In some embodiments, the coil may be configured to heat to about 150 degrees Fahrenheit. In some embodiments, the coil may be configured to apply the heat field for about half a second.

In some embodiments, the thermal fuse may be configured to stop the power supply when a temperature of 170 to 180 degrees is detected. In some embodiments, the thermal fuse may be configured to open the circuit of the heat sealing system such that the coil stops producing the heat field. In some embodiments, the thermal fuse may be configured to account for errors in the cooling device (e.g. the cooling device stops functioning), the coil (e.g. the coil does not stop producing the heat field), or a combination thereof. In some embodiments, the rest of the system is configured to continue functioning even if the thermal fuse is activated. In some embodiments, when the thermal fuse is activated, an alert may be sent to the user to prompt acknowledgement of the error in the heat sealing system.

In some embodiments, the power supply, the frequency generator, the thermal fuse, or a combination thereof may be disposed in an enclosure separate from the coil and the cooling system. In some embodiments, the heat sealing system may further comprise a current transducer operatively coupled to the frequency generator and the coil, configured to detect a current being delivered to coil from the frequency generator and confirm that the coil drew the correct amount of current. In some embodiments, if the current transducer detects that the coil did not draw enough power or drew too much power, the power supply may be shut down and the user may be notified of an error in the function of the coil.

In some embodiments, the system may further comprise a tray drawer system for accepting an array of bottles. In some embodiments, the tray drawer system may comprise a plurality of layers, each layer configured to hold a plurality of bottles. In some embodiments, the tray drawer system may further comprise a moving mechanism, configured to slide a layer to an outlet once filled by one or more bottles, and slide a subsequent layer into the original position of the layer such that the subsequent layer can accept bottles. In some embodiments, the tray drawer system may further comprise one or more sensors operatively coupled to the moving mechanism, configured to determine whether or not the tray is filled. In some embodiments, if the one or more sensors determine that the tray is filled, the moving mechanism may move the tray to the outlet. In some embodiments, the tray drawer system may allow for multiple orders to be filled at a time autonomously by the system of the present invention.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from a 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R.F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C#, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft® .NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

### Embodiments

The following embodiments are intended to be illustrative only and not to be limiting in any way.
Embodiment 1: An automated system for assembling an array of doses of a medication, the array of doses comprising one or a plurality of bottles each filled with a predetermined dose of the medication, said system comprising: a control center for receiving an order for an array of doses of the medication from an electronic medical record (EMR) system; a preparation system for filling the one or the plurality of bottles with the predetermined amount of medication and capping and labeling said one or plurality of bottles; and an assembly system for collating the one or the plurality of bottles with the predetermined amount of medication into the array.
Embodiment 2: The system of embodiment 1 further comprising a main software system operatively connected to the control center, the main software system is configured to receive the order for the array of doses of the medication from the EMR system and transmit a signal to the control center to provide instructions for preparing and assembling the array of doses of the medication.
Embodiment 3: The system of embodiment 2, wherein the main software system is configured to queue a plurality of orders for arrays of doses of medication and organize instructions to the control center for preparation and assembly of said plurality of order of arrays of doses of medication.
Embodiment 4: The system of embodiment 2 or 3, wherein the main software system removes patient personal information from the order sent by the EMR system and assigns a unique ID to each order prior to transmitting the signal to the control center of the automated system.
Embodiment 5: The system of any of embodiments 1-4, wherein the preparation system and assembly system are operatively connected to the control center so as to receive instructions therefrom.
Embodiment 6: The system of any of embodiments 1-5, wherein the preparation system comprises a dispensing system configured to dispense a predetermined dose of medication into bottles, a capping system configured to apply caps to bottles, and a labeling system configured to apply a label to each bottle.
Embodiment 7: The system of embodiment 6, wherein the dispensing system, capping system, and labeling system are operatively connected to the control center so as to receive instructions therefrom.
Embodiment 8: The system of any of embodiments 1-7, wherein the control center is operatively connected to the EMR system via a main software system configured to accept an order from the EMR system and provide instructions to the control center of the automated system to assemble the order.
Embodiment 9: The system of any of embodiments 1-8, wherein the medication is a liquid medication.
Embodiment 10: The system of any of embodiments 1-9, wherein the system is configured to allow a medication pump to be integrated.
Embodiment 11: An automated system for assembling an array of doses of a medication, the array of doses comprising one or a plurality of bottles each filled with a predetermined dose of the medication, said system comprising: a means for receiving an order for an array of doses of the medication; a means of filling one or a plurality of bottles with the predetermined amount of medication; and a means for collating the one or the plurality of bottles with the predetermined amount of medication into an array.
Embodiment 12: The system of embodiment 11, wherein the means for receiving an order for an array of doses of the medication, the means of filling one or a plurality of bottles with the predetermined amount of medication, and the means for collating the one or the plurality of bottles with the predetermined amount of medication into an array are housed in a frame enclosure.
Embodiment 13: The system of any of embodiments 11-12, wherein the system is configured to allow a medication pump to be integrated.
Embodiment 14: An automated system for assembling a collection of doses of a medication, said system comprising a control center for receiving an order for an array of doses of the medication from an electronic medical record (EMR) system; a dispensing system configured to dispense a predetermined dose of medication into bottles; a capping system configured to apply caps to bottles; a labeling system configured to apply a label to each bottle; and an order buffer system for collating the one or the plurality of bottles with the predetermined amount of medication into the array.
Embodiment 15: The system of embodiment 14, wherein the dispensing system, capping system, labeling system, and order buffer system are operatively connected to the control center so as to receive instructions therefrom to assemble the collection of doses of the medication.
Embodiment 16: The system of any of embodiments 14-15, wherein the control center is operatively connected to the EMR system via a main software system configured to accept an order from the EMR system and provide instructions to the control center of the automated system to assemble the order.
Embodiment 17: The system of any of embodiments 14-16, wherein the system is adapted to integrate a medication pump.
Embodiment 18: The system of any of embodiments 14-17, wherein the system comprises a dial table system comprising at least four slots therein for temporarily holding a medication bottle, wherein each of the at least four slots occupies either a loading position, a dispensing position, a capping position, or an unloading position, the dial table system rotates to move slots from one position to another.
Embodiment 19: The system of any of embodiments 14-18, wherein the labeling system is configured to obtain a bottle in the unloading position of the dial table.
Embodiment 20: The system of any of embodiments 14-19, wherein the capping system is configured to obtain a cap from a cap pickup location and securely attach the cap to a medication bottle in the capping position of the dial table system.
Embodiment 21: The system of any of embodiments 14-20, wherein the dispensing system is configured to aliquot a unit of medication from a reservoir through a dispenser into a medication bottle in the slot occupying the dispensing position of the dial table system.
Embodiment 22: The system of any of embodiments 14-21, wherein the system comprises a tray drawer system for accepting an array.
Embodiment 23: The system of embodiment 22, wherein the tray drawer provides a user access to the order array.
Embodiment 24: The system of any of embodiments 14-23, wherein the system comprises a bottle feeding system for obtaining a medication bottle from a bottle storage hopper.
Embodiment 25: The system of any of embodiments 14-24, wherein the system comprises a bottle feeding system for obtaining a medication bottle from a bottle storage hopper and directing the medication bottle to a slot occupying the loading position of the dial table system.
Embodiment 26: The system of any of embodiments 14-25, wherein the system comprises a cap feeding system for obtaining a cap from a cap storage hopper.
Embodiment 27: The system of any of embodiments 14-26, wherein the system comprises a cap feeding system for obtaining a cap from a cap storage hopper and directing the cap to a cap pickup location.
Embodiment 28: An automated system for assembling a collection of doses of a medication, said system comprising a control center for receiving an order for an array of doses of the medication from an electronic medical record (EMR) system; a dispensing system configured to dispense a predetermined dose of medication into bottles; a capping system configured to apply caps to bottles; a labeling system configured to apply a label to each bottle; and an order buffer system for collating the one or the plurality of bottles with the predetermined amount of medication into the array; and further comprising one or a combination of: a dial table system comprising at least four slots therein for temporarily holding a medication bottle, wherein each of the at least four slots occupies either a loading position, a dispensing position, a capping position, or an unloading position, the dial table system rotates to move slots from one position to another; a bottle feeding system for obtaining a medication bottle from a bottle storage hopper and directing the medication bottle to the slot occupying the loading position of the dial table system; a dispensing system for aliquoting a unit of medication from a reservoir through a dispenser into a medication bottle in the slot occupying the dispensing position of the dial table system, the dispensing system comprises a means of connecting a medication pump; a cap feeding system for obtaining a cap from a cap storage hopper and directing the cap to a cap pickup location; a capping system for obtaining a cap from the cap pickup location of the cap feeding system and securely attaching the cap to a medication bottle in the capping position of the dial table system; a labeling system for obtaining a bottle from the unloading position of the dial table system and labeling said bottle; an order buffer system for accepting bottles from the labeling system and organizing one or a plurality of bottles as an order array; or a tray drawer system for accepting an order array from the order buffer system and providing a user access to the order array.
Embodiment 29: The automated system of embodiment 28, wherein the automated system is operatively connected to a main software system, which is operatively connected to an electronic medical record (EMR) system.
Embodiment 30: The automated system of embodiment 29, wherein the main software system is a cloud based software system.
Embodiment 31: The automated system of embodiment 29 or 30, wherein the main software system is operatively connected to the EMR system via a clinic cloud software system.
Embodiment 32: The automated system of any of embodiments 29-31, wherein the main software system is configured to receive a medication order from the EMR system and provide instructions to the automated system for filling the medication order.
Embodiment 33: The system of any of embodiments 28-32, wherein the EMR system is accessible via a user interface on a healthcare professional's workstation.
Embodiment 34: The system of embodiment 33, wherein the user interface is operatively connected to a clinic's cloud based software.
Embodiment 35: The system of any of embodiments 29-34, wherein the main software system is operatively connected to an application for a healthcare professional's workstation.
Embodiment 36: A medication pump accessory system for automated assembling of an array of doses of a medication, said accessory system comprising: a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; and a capping system for securely attaching a cap to a medication bottle in the capping position of the dial table system.
Embodiment 37: The system of embodiment 36, wherein the control center is operatively connected to the EMR system via a main software system configured to accept an order from the EMR system and provide instructions to the control center of the automated system to assemble the order.
Embodiment 38: A medication pump accessory system for automated assembling of a medication order, said accessory system comprising: a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; a capping system for securely attaching the cap to a medication bottle in the capping position of the dial table system; and a labeling system for obtaining a bottle from the unloading position of the dial table system and labeling said bottle.
Embodiment 39: The system of embodiment 38, wherein the system is housed within a frame enclosure.
Embodiment 40: The system of any of embodiments 38-39, wherein the control center is operatively connected to the EMR system via a main software system configured to accept an order from the EMR system and provide instructions to the control center of the automated system to assemble the order.
Embodiment 41: A medication pump accessory system for automated assembling of a medication order, said accessory system comprising, in a frame enclosure: a control center for receiving an order for an array of doses of the medication directly or indirectly from an electronic medical record (EMR) system; a dial table system comprising at least four slots therein for temporarily holding a medication bottle, wherein each of the at least four slots occupies either a loading position, a dispensing position, a capping position, or an unloading position, the dial table system rotates to move slots from one position to another; a dispensing system for aliquoting a unit of medication from a reservoir through a dispenser into a medication bottle in the slot occupying the dispensing position of the dial table system, the dispensing system comprises a means of connecting a medication pump; a capping system for obtaining a cap from the cap pickup location of the cap feeding system and securely attaching the cap to a medication bottle in the capping position of the dial table system; a labeling system for obtaining a bottle from the unloading position of the dial table system and labeling said bottle; an order buffer system for accepting bottles from the labeling system and organizing one or a plurality of bottles as an order array; and a tray drawer system for accepting an order array from the order buffer system and providing a user access to the order array.
Embodiment 42: The system of embodiment 41, wherein the system is housed within a frame enclosure.
Embodiment 43: The system of any of embodiments 41-42, wherein the control center is operatively connected to the EMR system via a main software system configured to accept an order from the EMR system and provide instructions to the control center of the automated system to assemble the order.
Embodiment 44: A method of assembling an array of doses of a medication, said method comprising: providing an order for an array of doses of a medication from an electronic medical record (EMR) system to a main software system operatively connected to a control center of an automated system for assembling arrays of doses of medication, said automated system comprising: a control center for receiving an order for an array of doses of the medication from the electronic medical record (EMR) system via the main software system; a preparation system for filling the one or the plurality of bottles with the predetermined amount of medication and capping and labeling said one or plurality of bottles; and an assembly system for collating the one or the plurality of bottles with the predetermined amount of medication into the array; wherein providing the order from the EMR system triggers the main software system to send instructions to the control center of the automated system that allow for the automated system to prepare and assemble the array of doses of the medication.
Embodiment 45: The method of embodiment 44, wherein the main software system is a cloud based software system.
Embodiment 46: The method of embodiment 44, wherein the main software system is run on a local server.
Embodiment 47: The method of any of embodiments 44-46, wherein the main software system is operatively connected to the EMR system via a clinic cloud software system.
Embodiment 48: The method of any of embodiments 44-47, wherein the EMR system is accessible via a user interface on a healthcare professional's workstation.
Embodiment 49: The method of embodiment 48, wherein the user interface is operatively connected to a clinic's cloud based software.
Embodiment 50: The method of any of embodiments 44-49, wherein the main software system is operatively connected to an application on a healthcare professional's workstation.
Embodiment 51: A cap feeding system, said system comprising: a belt feeder comprising a vertically angled motor-driven belt with cleats spaced a distance apart, the cleats are adapted to support at least one cap; wherein a lower end of the belt feeder engages caps in a cap storage hopper such that as the belt rotates the cleats rise through caps in contact in the storage hopper to allow one or more caps to rest on the cleats as they rise, and caps move toward a top end of the belt feeder.
Embodiment 52: The system of embodiment 51, wherein the system further comprises the cap storage hopper.
Embodiment 53: The system of embodiment 51 or 52 further comprising a delivery guide for directing caps at the top end of the belt feeder to a cap pickup location.
Embodiment 54: A cap feeding system (200), said system comprising: a belt feeder (210) comprising a belt (220) that rotates around an elongated plate or elongated axis formed by a drive gear (262) operatively connected to a drive motor (260) and a belt wheel (242); a plurality of cleats (230) disposed on the belt (220) spaced a distance d1 apart; wherein a lower end of the belt feeder (210) is configured to engage caps housed in a cap storage hopper such that as the belt (220) rotates the cleats (230) rise and capture one or more caps thereon as the cleats (230) rise towards a top end of the belt feeder (210), wherein the belt feeder (210) is oriented at an angle a1 with respect to horizontal; and a delivery guide (280) that delivers caps (205) from at or near the top end of the belt feeder (210) to a cap pickup location (288).
Embodiment 55: The system of embodiment 54 further comprising a guide plate (250) disposed at or near the top end of the belt feeder (210), the guide plate (250) moves or causes movement of the caps off of the belt feeder (210).
Embodiment 56: The system of any of embodiments 54-55, wherein angle a1 is such that only caps in a correct orientation rise from the lower end of the belt feeder to the top end of the belt feeder and caps in an incorrect orientation fall off of the belt as the cleats rise.
Embodiment 57: The system of any of embodiments 54-56, wherein angle a1 is from 50 to 85 degrees.
Embodiment 58: The system of any of embodiments 54-57, wherein distance d1 allows caps to be loaded only along the cleats (230).
Embodiment 59: The system of any of embodiments 54-58, wherein distance d1 is from 40 to 60 mm.
Embodiment 60: The system of any of embodiments 54-59, wherein the guide plate (250) comprises a surface having an angle a2.
Embodiment 61: The system of embodiment 60, wherein angle a2 is from 40 to 50 degrees.
Embodiment 62: The system of any of embodiments 54-61, wherein the delivery guide (280) comprises a chute (282) through which caps travel from the belt feeder (210) to the cap pickup location (288), the chute (282) is configured to allow gravity to cause the cap to rotate upon exiting the belt feeder (210) and become oriented with its open end facing downwardly.
Embodiment 63: The system of any of embodiments 54-62 further comprising one or more sensors for detecting one or a combination of events: presence or absence of a cap at the cap pickup location; proper or improper orientation of a cap in the delivery guide or at the cap pickup location; a backup of caps in the delivery guide.
Embodiment 64: The system of embodiment 63 further comprising one or more sensors for detecting if the number of caps in the cap storage hopper is below a predetermined threshold.
Embodiment 65: The system of any of embodiments 54-64 further comprising a cap reject return component for capturing caps that fall off of the belt feeder to allow said caps to return to the cap storage hopper.
Embodiment 66: A bottle feeding system (300), said system comprising: a belt feeder comprising a vertically angled motor-driven belt with cleats spaced a distance apart, the cleats are adapted to support at least one bottle; wherein a lower end of the belt feeder engages bottles in a bottle storage hopper such that as the belt rotates the cleats rise through bottles in contact in the bottle storage hopper to allow one or more bottles to rest on the cleats as they rise, and bottles move toward a top end of the belt feeder.
Embodiment 67: A bottle feeding system (300), said system comprising: a belt feeder (310) comprising a belt (320) that rotates around an elongated plate or elongated axis formed by a drive gear (362) operatively connected to a drive motor (360) and a belt wheel (3242); a plurality of cleats (330) disposed on the belt (320) spaced a distance d2 apart; wherein a lower end of the belt feeder (310) is configured to engage bottles housed in a bottle storage hopper such that as the belt (320) rotates the cleats (330) rise and capture one or more bottles thereon as the cleats (330) rise towards a top end of the belt feeder (310), wherein the belt feeder (310) is oriented at an angle a1 with respect to horizontal; and a bottle delivery guide (380) that delivers bottles (305) from the belt feeder (310) to a bottle pickup location (388).
Embodiment 68: The system of embodiment 67, wherein the system is configured to deliver one bottle per cleat to the bottle pickup location (388).
Embodiment 69: The system of embodiment 67 or 68 further comprising a guide plate (350) disposed at or near the top end of the belt feeder (310), the guide plate (350) moves or causes movement of the bottles to a side edge of the belt feeder (310).
Embodiment 70: The system of any of embodiments 67-69, wherein the system is configured such that bottles falling off the belt feeder (310) return to a bottle storage hopper via a return chute (390).
Embodiment 71: The system of any of embodiments 67-70, wherein one bottle per cleat can pass the guide plate (350) and reach an exit point.
Embodiment 72: The system of any of embodiments 67-71, wherein the system further comprises a bottle delivery guide (380) through which bottles leaving the belt feeder (310) travel to a bottle pickup location (388).
Embodiment 73: The system of any of embodiments 67-72 further comprising a pusher wheel system (362) for pushing bottles off of the belt feeder (310) at an exit point, the bottles travel through a bottle delivery guide (380) to a bottle pickup location (388).
Embodiment 74: The system of embodiment 73, wherein the pusher wheel system (362) comprises a rotating pusher wheel (363) operatively connected to a pusher wheel motor (365).
Embodiment 75: The system of embodiment 73 or 74, wherein the pusher wheel system (362) comprises a flexible drive spring (367) which helps move the bottle into the delivery guide as the bottle passes the rotating pusher wheel (363).
Embodiment 76: The system of any of embodiments 73-75 wherein the speed of the drive motor (360) and pusher wheel system (362) can be adjusted to accommodate a particular rate of bottle delivery.
Embodiment 77: The system of any of embodiments 67-76, wherein the belt (320) is oriented at an angle b1 with respect to horizontal.
Embodiment 78: The system of embodiment 77, wherein angle b1 is from 50 to 85 degrees.
Embodiment 79: The system of any of embodiments 67-78, wherein distance d2 is from 1 to 3 inches.
Embodiment 80: The system of any of embodiments 67-79, wherein the guide plate (350) comprises a surface having an angle b2.
Embodiment 81: The system of embodiment 80, wherein angle a2 is from 10 to 60 degrees.
Embodiment 82: The system of any of embodiments 67-81, wherein the bottle delivery guide (380) comprises a gravity chute (381) and a selector (340) disposed at an end of the gravity chute (381), wherein gravity causes the bottles to travel through the gravity chute (381) to the selector (340).
Embodiment 83: The system of embodiment 82, wherein the delivery guide (380) comprises a first bottle sensor (387) in the chute (381) for detecting a backup of bottles.
Embodiment 84: The system of any of embodiments 82-83, wherein the selector (340) comprises a rotator wheel (341) operatively connected to a rotator wheel drive motor (346), the rotator wheel (241) rotates bottles clockwise or counterclockwise to orient a bottle in a correct orientation and subsequently delivers bottles through a second chute (349).
Embodiment 85: The system of any of embodiments 82-84 wherein the selector (340) further comprises a first selector sensor (342) for detecting if the bottle is in the correct position in the rotator wheel (341) and a second selector sensor (343) for detecting orientation of the bottle.
Embodiment 86: The system of any of embodiments 67-85 further comprising one or more sensors for detecting one or a combination of events: presence or absence of a bottle at the bottle pickup location; proper or improper orientation of a bottle in the delivery guide or at the bottle pickup location; a backup of bottles in the delivery guide.
Embodiment 87: The system of any of embodiments 67-86 further comprising one or more sensors for detecting if the number of bottles in the bottle storage hopper is below a predetermined threshold.
Embodiment 88: An automated system for assembling an array of doses of a medication, said system comprising: a dial table system comprising a rotating dial having a plurality of slots therein for accepting bottles, the plurality of slots comprises a loading position, a dispensing position, a capping position, and an unloading position, the dial table is operatively connected to a motor and is rotatable in at least a first direction around an axis to allow movement of bottes from one position to a next position.
Embodiment 89: The system of embodiment 88, wherein the rotating dial (450) comprises a first slot, a second slot, a third slot, and a fourth slot.
Embodiment 90: The system of embodiment 89, wherein the slots can occupy one of: a first position for allowing a bottle to be loaded; a second position for allowing medication to be dispensed into the bottle; a third position for allowing the bottle to be capped; and a fourth position for allowing the bottle to be unloaded.
Embodiment 91: The system of embodiment 89 or 90, wherein the rotating dial (450) can rotate in at least a first direction such that (i) a the first slot occupies the first position, the second slot occupies the second position, the third slot occupies the third position, and the fourth slot occupies the fourth position; or (ii) a the first slot occupies the second position, the second slot occupies the third position, the third slot occupies the fourth position, and the fourth slot occupies the first position; or (iii) a the first slot occupies the third position, the second slot occupies the fourth position, the third slot occupies the first position, and the fourth slot occupies the second position; or (iv) a the first slot occupies the fourth position, the second slot occupies the first position, the third slot occupies the second position, and the fourth slot occupies the third position.
Embodiment 92: The system of any of embodiments 88-91, wherein the dial table is rotatably attached to a support platform.
Embodiment 93: The system of any of embodiments 88-92, wherein the dial table is configured to allow a bottle to be inserted into the first position with its opening facing up.
Embodiment 94: The system of any of embodiments 88-93, wherein the dial table is configured to allow medication to be dispensed from a dispenser into a bottle in the second position.
Embodiment 95: The system of any of embodiments 88-94, wherein the dial table is configured to allow a bottle in the third position to be capped.
Embodiment 96: The system of any of embodiments 88-95, wherein the dial table is configured to allow a bottle in the fourth position to be retrieved.
Embodiment 97: The system of any of embodiments 88-96 further comprising one or more sensors for detecting one or a combination of events: presence or absence of a bottle in a slot in the first position; proper or improper positioning of a bottle in a slot in the first position; presence or absence of a bottle in a slot in the second position; presence or absence of a bottle in a slot in the third position; presence or absence of a bottle in a slot in the fourth position.
Embodiment 98: A dispensing system (500) for dispensing a liquid medication into a medication bottle, said system comprising: a reservoir (530) for holding liquid medication; a dispenser arm (510) with an exit port through which medication is aliquoted, the dispenser arm (510) is fluidly connected to the reservoir (530); a first tube having a first end fluidly connected to the reservoir (530) and a second end adapted to operatively connect to a medication pump such that medication can be pumped from the reservoir (530); and a second tube having a first end fluidly connected to the dispenser arm (510) and a second end adapted to operatively connect to a medication pump such that medication can be dispensed via the dispenser arm (510).
Embodiment 99: The system of embodiment 98 further comprising the medication pump integrated therein.
Embodiment 100: The system of any of embodiments 98-99, wherein the system is configured to dispense medication into bottles disposed in a dial table system (400).
Embodiment 101: The system of any of embodiments 98-100, wherein the dispenser arm (510) is operatively connected to a motor (560) and can occupy and move between two or more positions.
Embodiment 102: The system of embodiment 101, wherein the dispenser arm (510) can occupy a first position wherein the dispenser arm (510) is positioned to dispense medication into a bottle held in a dial table system (400); and a second position wherein the dispenser arm (510) is positioned to dispense a fluid into a calibration flask (540).
Embodiment 103: The system of embodiment 102, wherein the dispenser arm (510) can occupy a third position wherein dispenser arm (510) is positioned to dispense a fluid into a waste container or waste funnel (542).
Embodiment 104: The system of any of embodiments 98-103, wherein the system is operatively connected to a control center.
Embodiment 105: The system of embodiment 104, wherein the system comprises a mechanism for operatively connecting a medication pump to the control center, the control center is configured to send instructions to the medication pump for pumping an appropriate volume of medication from the reservoir (530) to and through the dispenser arm (510).
Embodiment 106: The system of any of embodiments 98-105 further comprising one or more sensors for detecting proper or improper position of the dispenser arm (510).
Embodiment 107: An automated system for assembling an array of doses of a medication, said system comprising a capping system for receiving a cap, positioning said cap so as to allow attachment to a bottle, and attaching said cap to a bottle.
Embodiment 108: The system of embodiment 107, wherein the capping system obtains the cap from a cap feeding system.
Embodiment 109: The system of embodiment 107 or 108, wherein the bottle is immobilized in a dial table when the capping system attaches the cap to the bottle.
Embodiment 110: The system of any of embodiments 107-109, wherein the capping system (600) comprises a pick and place system (610) for obtaining a cap and positioning the cap above the bottle and attaching the cap to the bottle; and a bottle clamp system (630) for immobilizing the bottle while the cap is being attached to the bottle by the pick and place system (610).
Embodiment 111: The system of embodiment 110, wherein the bottle clamp system (630) comprises a bottle clamp cylinder (634) and a bottle clamp motor (363) operatively connected to the bottle clamp cylinder (634), the bottle clamp motor (363) drives motion of the bottle clamp cylinder (634) between an engaged position and a disengaged position, wherein in the engaged position, the bottle clamp cylinder (634) clamps and immobilizes the bottle, and when the bottle clamp cylinder (643) is in the disengaged position the bottle is not immobilized.
Embodiment 112: The system of embodiment 111, wherein the bottle clamp cylinder (634) comprises a gripping component for providing a grip on the bottle for immobilization.
Embodiment 113: The system of any of embodiments 110-112, wherein the pick and place system (610) comprises a rotary gripper (620) for retrieving a cap from a cap pickup location (288) and threading the cap on the bottle, the rotary gripper (620) is operatively connected to at least one motor (616).
Embodiment 114: The system of embodiment 113, wherein the rotary gripper (620) rotary gripper (620) comprises a torque feedback system for controlled threaded engagement between the cap and bottle, wherein the rotary gripper (620) is configured such that when activated the rotary gripper (620) rotates and and lowers the cap at a predetermined rate to allow threads of the cap to engage threads of the bottle until the rotary gripper (620) achieves a threshold torque value, a threshold distance, or both a threshold torque value and a threshold distance.
Embodiment 115: The system of embodiment 114, wherein the rotary gripper (620) is configured to disengage from the cap when the rotary gripper (620) achieves the threshold torque value, the threshold distance, or both the threshold torque value and the threshold distance.
Embodiment 116: The capping system of any of embodiments 107-115 further comprising one or more sensors for detecting one or a combination of events: proper or improper positioning of the bottle; proper or improper cap attachment; proper or improper positioning of the clamp cylinder.
Embodiment 117: The capping system of any of embodiments 107-116, wherein the system is configured to divert poorly capped bottles to a quarantine location.
Embodiment 118: An automated system for assembling an array of doses of a medication, said system comprising a labeling system (700) for labeling filled medication bottles.
Embodiment 119: The system of embodiment 118, wherein the labeling system (700) comprises a robotic arm system (710) for obtaining a bottle in a bottle pickup position and moving the bottle between one or more positions; and a printing system (750) for printing and applying a label on the bottle and monitoring the label thereon.
Embodiment 120: The system of embodiment 119, wherein the robotic arm system (710) is configured to obtain a bottle from the bottle pickup position in a dial table system (400) and move the bottle to a printing position (740) for labeling.
Embodiment 121: The system of embodiment 119 or 120, wherein the robotic arm system (710) comprises a gripper (720) that can move between at least an engaged configuration and a disengaged configuration, wherein in the engaged configuration the gripper (720) grips a bottle and in the disengaged configuration the gripper (720) releases the bottle.
Embodiment 122: The system of embodiment 121, wherein the robotic arm system (710) is configured to move the gripper (720) between at least the bottle pickup position and the printing position (740).
Embodiment 123: The system of embodiment 122, wherein the robotic arm system (710) is configured to move the gripper (720) to the bottle pickup position and the engaged configuration to grip a bottle, then move the gripper (720) to the printing position (740) and the disengaged configuration to release the bottle.
Embodiment 124: The system of embodiment 123, wherein the robotic arm system (710) is configured to maintain the gripper (720) at the printing position (740) and move the gripper (720) to the engaged configuration to grip the bottle, then move the gripper (720) to an order buffer position and the disengaged configuration to release the bottle.
Embodiment 125: The system of embodiment 123, wherein the robotic arm system (710) is configured to maintain the gripper (720) at the printing position (740) and move the gripper (720) to the engaged configuration to grip the bottle, then move the gripper (720) to a quarantine position and the disengaged configuration to release the bottle.
Embodiment 126: The system of embodiment 125, wherein the robotic arm system (710) is configured to move the gripper (720) from the order buffer position to the bottle pickup position.
Embodiment 127: The system of embodiment 126, wherein the robotic arm system (710) is configured to move the gripper (720) from the quarantine position to the bottle pickup position.
Embodiment 128: The system of any of embodiments 118-127, wherein the printing system (750) comprises a printer and a label application machine, the printer prints a label and the label application attaches the label to a medication bottle.
Embodiment 129: The system of embodiment 128, wherein the printing system (750) comprises a sensor (782) for detecting presence or absence of a bottle in the printing position (740).
Embodiment 130: The system of embodiment 128 or 129, wherein the label comprises a barcode.
Embodiment 131: The system of any of embodiments 128-130 wherein the printing system (750) comprises a scanner for scanning barcodes on labels.
Embodiment 132: The system of any of embodiments 128-131, wherein the label (306) features a tab (307) extending from an edge of the label (306) but removable via perforations or other destructive mechanisms.
Embodiment 133: An automated system for assembling an array of doses of a medication, said system comprising: an order buffer system (800) for assembling a medication order from one or a plurality of filled medication bottles.
Embodiment 134: The system of embodiment 133, wherein the order buffer system (800) is configured to store a medication order until it is retrieved from the system.
Embodiment 135: The system of embodiment 133 or 134, wherein the order buffer system (800) is operatively connected to a labeling system (700) which labels medication bottles and a tray drawer system (900) which provides access to a medication order.
Embodiment 136: The system of any of embodiments 133-135, wherein a medication order comprises an array (805) of medication bottles.
Embodiment 137: The system of any of embodiments 133-136, wherein the order buffer system (800) comprises an array platform (810) for supporting one or a plurality of filled medication bottles placed thereon.
Embodiment 138: The system of embodiment 137, wherein one or more guide rails (870) surround the array platform (810) to prevent bottles from falling off of the array platform (810).
Embodiment 139: The system of any of embodiments 137-138 further comprising a mechanism for moving the array (805) to a tray (910) of a tray drawer system (900).
Embodiment 140: The system of embodiment 139, wherein the mechanism for moving the array (805) comprises a pusher arm system (920), comprising an elongated arm (822) operatively connected to a motor (860) which drives movement of the elongated arm (822) between at least a first position (826) wherein the elongated arm (822) is retracted behind the array (805) and a second position (828) wherein the elongated arm (822) has moved toward the tray (910) of the tray drawer system (900) sufficiently to move the array (805) completely onto the tray (910).
Embodiment 141: The system of embodiment 140, wherein the pusher arm system (820) further comprises a drive belt (862) operatively connected to the motor (860) and rotatable around an axis, wherein the motor (860) drives rotation of the drive belt (862) about the axis in a first direction and a second direction opposite the first direction.
Embodiment 142: The system of embodiment 141, wherein the pusher arm system (820) further comprises a support carriage (866) operatively connected to the drive belt (862) and operatively connected to the elongated arm (822) such that rotation of the drive belt (862) via the motor (860) causes the support carriage (866) and elongated arm (822) to move from the first position (826) to the second position (828) and vice versa.
Embodiment 143: The system of embodiment 142, wherein the support carriage (866) rides on carriage guide rails (864) that stabilize the elongated arm (822) as it progresses between at least the first position (826) and the second position (828).
Embodiment 144: The system of any of embodiments 133-143 further comprising one or more sensors for detecting proper or improper positioning of the elongated arm (822).
Embodiment 145: The system of any of embodiments 134-144, wherein the order buffer system (800) comprises one or more sensors for providing status of the array (805).
Embodiment 146: The system of embodiment 145, wherein the order buffer system (800) comprises one or more sensors for detecting a position of the elongated arm (822).
Embodiment 147: The system of any of embodiments 133-146, wherein the order buffer system (800) has the capacity to hold at least 28 medication bottles.
Embodiment 148: An automated system for assembling an array of doses of a medication, said system comprising, within a frame enclosure: a tray drawer system (900) for accepting a completed medication order in the form of an array (805) of medication bottles.
Embodiment 149: The system of embodiment 148, wherein the tray drawer system (900) is operatively connected to an order buffer system (800).
Embodiment 150: The system of embodiment 148 or 149, wherein the tray drawer system (900) comprises a tray drawer (920) with an inner cavity adapted to accept an order array (805); a removable tray (910) for supporting an array (805); and a door (916) disposed in the tray drawer (920) that can move between at least an open position which provides access to the inner cavity of the tray drawer (920) and a closed position which prevents access to the inner cavity of the tray drawer (920).
Embodiment 151: The system of embodiment 150, wherein the tray drawer (920) comprises a transparent area (928) for providing visual access to the tray drawer (920).
Embodiment 152: The system of embodiment 150 or 151, wherein the tray drawer (920) extends from the frame enclosure (110).
Embodiment 153: The system of any of embodiments 150-152, wherein the door (916) can be temporarily secured in the closed position via a closure component (918).
Embodiment 154: The system of any of embodiments 148-153, wherein the tray drawer system (900) comprises a tray door sensor (930) for determining whether or not the drawer (916) is in the open position or closed position.
Embodiment 155: The system of any of embodiments 148-154, wherein the tray drawer system (900) further comprises a door lock (950) for temporarily preventing the door from moving from the closed position to the open position.
Embodiment 156: The system of any of embodiments 148-155, wherein the tray drawer system (900) comprises a clearance sensor (840) which scans the inner cavity of the tray drawer (920) to determine if the tray drawer (920) is clear of bottles.
Embodiment 157: The system of any of embodiments 148-156, wherein the tray drawer system (900) comprises tray sensors (940) in the tray drawer (920) for determining if the tray (910) is in a proper position or determining if more than one tray is present in the tray drawer (920).
Embodiment 158: An automated system for assembling doses of a medication, said system comprising: a control system (1000) operatively connected to at least a dispensing system for dispensing a volume or dose of medication into a bottle and operatively connected to a main software system (1050), the main software system (1050) is configured to send instructions to the control system (1000) for activating the dispensing system to fill a medication order.
Embodiment 159: The system of embodiment 158, wherein the main software system (1050) is operatively connected to an electronic medical record (EMR) (1090).
Embodiment 160: The system of embodiment 158 or 159, wherein the main software system (1050) is directly operatively connected to an electronic medical record (EMR) (1090).
Embodiment 161: The system of embodiment 158 or 159, wherein the main software system (1050) is operatively connected to the EMR via a EMR cloud-based software system (1040).
Embodiment 162: The system of any of embodiments 158-161, wherein the EMR (1090) is operable via a healthcare professional's workstation (1010).
Embodiment 163: The system of any of embodiments 158-162, wherein the main software system (1050) is operatively connected to a system application (1080) operable via a healthcare professional's workstation (1010).
Embodiment 164: The system of embodiment 163, wherein the system application (1080) is configured to provide one or a combination of: a patient-associated ID number, an order number, dispensing status, and a dose count.
Embodiment 165: The system of any of embodiments 158-164, wherein an order is created with the EMR (1090), the order is communicated through the EMR cloud-based software system (1040) and further to the main software system (1050).
Embodiment 166: The system of any of embodiments 158-165, wherein the control system (1000) is operatively connected to one or a combination of a cap feeding system (200), a bottle feeding system (300), a dial table system (400), a dispensing system (500), a capping system (600), a labeling system (700), an order buffer system (800), and a tray drawer system (900).
Embodiment 167: The system of any of embodiments 158-166, wherein the control system (1000) is operatively connected to a medication pump (550).
Embodiment 168: The system of any of embodiments 158-167, wherein the control system (1000) is operatively connected to a label printer (750).
Embodiment 169: The system of any of embodiments 158-168, wherein the control system (1000) is operatively connected to a labeling system (700).
Embodiment 170: The system of any of embodiments 158-169, wherein the control system (1000) is operatively connected to a local database.
Embodiment 171: The system of any of embodiments 158-170, wherein the EMR cloud-based software system (1040) is configured to receive an order from the EMR (1090) and replace patient information with a unique patient ID.
Embodiment 172: The system of any of embodiments 158-171, wherein the EMR is configured to replace patient information with a unique patient ID prior to sending the order to the main software system (1050).
Embodiment 173: The system of embodiment 172, wherein the main software system (1050) is configured to receive an order associated with a unique patient ID.
Embodiment 174: The system of embodiment 173, wherein the main software system (1050) processes the order and sends instructions to the control system (1000) to fill the order.
Embodiment 175: The system of embodiment 174, wherein the control system (1000) sends status information to the main software system (1050), whereupon the status information is transmitted to the system application (1080) on the workstation (1010).
Embodiment 176: The system of any of embodiments 158-175, wherein the control system (1000) is configured to send completed status information to the main software system (1050) upon order completion.
Embodiment 177: The system of embodiment 176, wherein the main software system (1050) is configured to send completed status information to the clinic software system (1040), the clinic software system (1040) is configured to send completed status information to the EMR (1090).
Embodiment 178: A safe system (1100) for remote automated dispensation of doses of a medication, the safe system (1100) comprising: a safe housing (1110); a dispense port (1112) disposed in the safe housing (1100); one or more dosage containers (1120) containing a medication; and one or more dispense mechanisms operatively coupled to the one or more dosage containers (1120) and the dispense port (1112).
Embodiment 179: The safe system (1100) of embodiment 178, wherein the one or more dispense mechanisms are operatively connected to the one or more dosage containers (1120) via a pump.
Embodiment 180: The safe system (1100) of embodiment 178, wherein the one or more dispense mechanisms are configured to integrate a pump for operative connection to the one or more dosage containers.
Embodiment 181: The safe system (1100) of any of embodiments 178-180 further comprising one or more pump mechanisms (1130) operatively coupled to the dosage container or containers (1120) and the dispense port (1112).
Embodiment 182: The safe system (1100) of any of embodiments 178-181, wherein the pump mechanism (1130) is operatively connected to the dosage container (1120) by a suction connection.
Embodiment 183: The safe system (1100) of embodiment 182, wherein the suction connection comprises a switch to control suction, wherein the switch is actuated by the computer actuation component (1140).
Embodiment 184: The safe system (1100) of any of embodiments 178-183 further comprising a computer actuation component (1140) operatively coupled to the one or more pump mechanisms (1130), wherein actuating the computer actuation component causes the one or more pump mechanisms (1130) to draw a controlled substance dose from the dosage container or containers (1120), and direct the controlled substance dose to the packaging mechanism, then to the dispense port (1112).
Embodiment 185: The safe system (1100) of embodiment 184 further comprising a computer actuation component (1140) operatively coupled to the one or more dispense mechanisms (1130), wherein actuating the computer actuation component causes the one or more dispense mechanisms (1130) to collect a controlled substance dose from the dosage container or containers (1120), and direct the controlled substance dose to the packaging mechanism, then to the dispense port (1112).
Embodiment 186: A method for automated dispensation of doses of a medication, the method comprising: providing instructions to a safe system (1100) comprising according to any of embodiments 178-185, wherein the safe system (1100) is configured to prepare one or more doses of the medication.
Embodiment 187: The method of embodiment 186, wherein the medication is a controlled substance.
Embodiment 188: The method of embodiment 187, wherein the controlled substance comprises methadone, buprenorphine, extended-release naltrexone, or a combination thereof in liquid, tablet, or sublingual form.
Embodiment 189: An opioid treatment booth (1300) for providing an interface between a user and a healthcare professional, said booth (1300) comprising: a user interface chamber (1400) with an interior space configured for a user's presence therein; an interface system (1420); a treatment delivery component (1520) configured to obtain and deliver one or more treatment doses from a storage container (1510); and a biometric input processing component (1410) capable of accepting a biometric input from the user as a means of authorization, the biometric input processing component (1410) is operatively connected to the interface system (1420), wherein when the biometric input processing component (1410) authorizes the biometric input of the user, a signal is sent to activate the treatment delivery component (1520) to deliver the one or more treatment doses.
Embodiment 190: The opioid treatment booth (1300) of embodiment 189 further comprising an input component (1422) capable of allowing the user to communicate with the healthcare professional and a display component (1424) capable of allowing the healthcare professional to communicate with the user.
Embodiment 191: The opioid treatment booth (1300) of embodiment 189 or 190 further comprising a treatment exchange component (1430).
Embodiment 192: The opioid treatment booth (1300) of embodiment 191, wherein the treatment exchange component (1430) is capable of accepting a treatment dose from a treatment containment chamber (1500).
Embodiment 193: The opioid treatment booth (1300) of any of embodiments 189-192, wherein the biometric input processing component (1410) further accepts a user ID number in addition to the biometric input as the means of authorization.
Embodiment 194: The opioid treatment booth (1300) of any of embodiments 189-193, wherein the biometric input is a fingerprint, a facial scan, a retinal scan, a palm-vein scan, or a combination thereof.
Embodiment 195: The opioid treatment booth (1300) of any of embodiments 189-194, wherein the input component (1422) comprises a touch screen, a keyboard, a microphone, a standard camera, a thermal image, or a combination thereof.
Embodiment 196: The opioid treatment booth (1300) of any of embodiments 189-195, wherein the display component (1424) comprises a screen, a speaker, or a combination thereof.
Embodiment 197: The opioid treatment booth (1300) of any of embodiments 189-196, wherein the healthcare professional, while connecting to the opioid treatment booth (1300), is able to view the user's ID number, location, wait time, and notifications.
Embodiment 198: The opioid treatment booth (1300) of any of embodiments 189-197, wherein the treatment delivery component (1520) delivers the treatment dose by dispensing, coupled to a pump (1710), the treatment dose into a container in a fluid path to be transferred to the user through the treatment exchange component (1430).
Embodiment 199: The opioid treatment booth (1300) of any of embodiments 189-198, wherein the healthcare professional is able to view a total number of treatment doses contained in the treatment storage container (1510).
Embodiment 200: The opioid treatment booth (1300) of any of embodiments 189-199 further comprising a robotic arm (1600) disposed in the treatment containment chamber (1500) connected to a computing component comprising a processor capable of executing computer-readable instructions and a memory component comprising computer readable instructions for: moving the treatment dose from the treatment delivery component (1520) to the treatment exchange component (1430); removing an unused treatment dose from the treatment delivery component (1520); placing the unused treatment dose into a safe (1610) or quarantine zone in the treatment containment chamber (1500); and pouring the unused treatment dose into the disposal component (1700) and adjusting the total number of treatment doses contained in the treatment storage container (1510) accordingly.
Embodiment 201: The opioid treatment booth (1300) of any of embodiments 189-200, wherein the healthcare professional is able to access the treatment containment chamber (1500) for restocking medication.
Embodiment 202: The opioid treatment booth (1300) of any of embodiments 189-201, wherein the treatment storage container (1510) comprises a plurality of individual treatment dose containers, wherein actuating the treatment delivery component (1520) causes the pump (1710) to extract the treatment dose from an individual treatment dose container, wherein subsequent actuations of the treatment delivery component (1520) cause the pump (1710) to extract from a different individual treatment dose container.
Embodiment 203: The opioid treatment booth (1300) of any of embodiments 189-202, wherein the treatment storage container (1510) comprises a single vat containing the plurality of treatment doses, wherein actuation of the treatment delivery component (1520) causes the pump (1710) to extract the treatment dose from the single vat.

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A medication pump accessory system for automated assembling of an array of doses of a medication, said accessory system comprising:
a. a control center (190) configured to receive an order for an array of doses of the medication directly or indirectly from an electronic medical record (EMR) system;
b. a dial table system (400) comprising at least four slots therein, configured to temporarily hold a medication bottle, wherein each of the at least four slots occupies either a loading position, a dispensing position, a capping position, or an unloading position, the dial table system (400) further configured to rotate to move slots from one position to another;
c. a dispensing system (500) configured to aliquote a unit of medication from a reservoir through a dispenser into the medication bottle in the slot occupying the dispensing position of the dial table system (400), the dispensing system (500) comprises a means of connecting a medication pump; and
d. a capping system (600) configured to securely attach a cap to the medication bottle in the capping position of the dial table system (400).

2. The system of claim 1 further comprising a bottle feeding system (300) configured to obtain a medication bottle from a bottle storage hopper and directing the medication bottle to a slot occupying the loading position of the dial table system (400), the bottle feeding system (300) comprising:
a. a belt feeder (310) comprising a belt (320), configured to rotate around an elongated plate or elongated axis formed by a drive gear (362) operatively connected to a drive motor (360) and a belt wheel (3242);
b. a plurality of cleats (330) disposed on the belt (320) spaced a distance d2 apart;
wherein a lower end of the belt feeder (310) is configured to engage bottles housed in a bottle storage hopper such that as the belt (320) rotates the cleats (330) rise and capture one or more bottles thereon as the cleats (330) rise towards a top end of the belt feeder (310), wherein the belt feeder (310) is oriented at an angle a1 with respect to horizontal; and
c. a bottle delivery guide (380) configured to deliver bottles (305) from the belt feeder (310) to the slot occupying the loading position of the dial table system (400).

3. The system of claim 2 further comprising:
a. an excess bottle return chute (312) disposed adjacent to the belt feeder (310), configured to deliver bottles to the bottle storage hopper;
b. a first guide plate (314) disposed at or near a bottom end of the belt feeder (310), configured to cause movement of bottles in an improper configuration on the belt (320) to the excess bottle return chute (312); and
c. a second guide plate (316) disposed at or near a top end of the belt feeder (310), configured to cause movement of the bottles (305) to the bottle delivery guide (380).

4. The system of claim 3, wherein the first guide plate (314) comprises an angle a4 with respect to vertical, wherein the second guide plate (316) comprises an angle a5 with respect to vertical;
wherein the angle a4 determines a speed at which the bottles in the improper configuration are moved to the excess bottle return chute (312);
wherein the angle a5 determines a speed at which the bottles (305) are moved to the bottle delivery guide (380).

5. The system of claim 3, wherein one bottle per cleat can pass the second guide plate (316) and reach an exit point.

6. The system of claim 3, wherein the first guide plate (314) and the second guide plate (316) are integrated into a double-guide plate.

7. The system of claim 2, wherein the plurality of cleats (330) comprise a first set of cleats (332) and a second set of cleats (334);
wherein each cleat of the first set of cleats (332) comprises a width w3 extending from the belt (320), wherein the first set of cleats (332) are suitable for supporting the bottles (205);
wherein each cleat of the second set of cleats (334) comprises a width w4 extending from the belt (320), wherein the second set of cleats (334) are unsuitable for supporting the bottles (305);
wherein the second set of cleats (334) are alternately disposed between the first set of cleats (332).

8. The system of claim 1 further comprising a clamping system (1200) configured to hold the medication bottle in place in a slot of the dial table system (400), the clamping system (1200) comprising:
a. a base mount (1210) having a front end and a back end;
b. a clamping arm (1220) having a front end and a back end, slidably disposed on the base mount (1210);
c. an insert (1230) disposed at the front end of the clamping arm (1220), the insert (1230) comprising a concave curved shape matching a curvature of the medication bottle;
d. a motor mount (1240) operatively coupled to the back end of the base mount (1210); and
e. a clamping motor mechanism (1250) comprising a motor (1252) operatively coupled to the motor mount (1240), and a shaft (1254) operatively coupled to the motor (1252) and the back end of the clamping arm (1220), wherein the clamping motor mechanism (1250) is configured to extend the shaft (1254) such that the clamping arm (1220) applies pressure to the medication bottle, and retract the shaft (1254) such that the clamping arm (1220) does not contact the medication bottle.

9. The system of claim 8 further comprising one or more position sensors (1260) operatively coupled to the base mount (1210) of the clamping system (1200), configured to detect a distance between the insert (1230) and the medication bottle, and stop the motor (1252) when the distance between the insert (1230) and the medication bottle reaches a threshold.

10. The system of claim 8, wherein each slot of the at least four slots of the dial table system (400) comprises a dial table insert (1270) disposed in the slot, the dial table insert (1270) having a concave curved shape matching a curvature of the medication bottle.

11. The system of claim 1, wherein the cap comprises a foil layer coupled to an interior of the cap by a meltable seal, wherein the system further comprises a heat sealing system (1300) operatively coupled to the unloading position of the dial table system (400), configured to melt the meltable seal of the cap such that the foil layer is applied to the medication bottle, the heat sealing system (1300) comprising:
a. a frequency generator (1310), configured to generate a frequency;
b. a power supply (1320) operatively coupled to the frequency generator (1310), configured to supply power to the frequency generator (1310); and
c. a coil (1330) operatively coupled to the frequency generator (1310), configured to accept the frequency and generate a heat field such that the meltable seal melts such that the foil layer is applied to the medication bottle.

12. The system of claim 11 further comprising a cooling device (1340) operatively coupled to the coil (1330), configured to cool the coil (1330) after the heat field is generated.

13. The system of claim 11 further comprising a thermal fuse (1350) operatively coupled to the coil, configured to detect a temperature of the coil (1330), and shut down the power supply (1320) if the temperature of the coil (1330) exceeds a temperature threshold.

14. The system of claim 11, wherein the capping system (600) is further configured to overtorque the cap onto the medication bottle.

15. A medication pump accessory system for automated assembling of a medication order, said accessory system comprising:
a. a control center (190) for receiving an order for an array of doses of the medication directly or indirectly from an electronic medical record (EMR) system;
b. a dial table system (400) comprising at least four slots therein for temporarily holding a medication bottle, wherein each of the at least four slots occupies either a loading position, a dispensing position, a capping position, or an unloading position, the dial table system (400) rotates to move slots from one position to another;
c. a dispensing system (500) for aliquoting a unit of medication from a reservoir through a dispenser into a medication bottle in the slot occupying the dispensing position of the dial table system (400), the dispensing system (500) comprises a means of connecting a medication pump;
d. a capping system (600) for securely attaching a cap to a medication bottle in the capping position of the dial table system (400);
e. a labeling system for obtaining a bottle from the unloading position of the dial table system (400) and labeling said bottle;
f. a bottle feeding system (300) configured to obtain a medication bottle from a bottle storage hopper and directing the medication bottle to a slot occupying the loading position of the dial table system (400);
g. a clamping system (1200) configured to hold the medication bottle in place in a slot of the dial table system (400);
wherein the cap comprises a foil layer coupled to an interior of the cap by a meltable seal; and
h. a heat sealing system (1300) operatively coupled to the unloading position of the dial table system (400), configured to melt the meltable seal of the cap such that the foil layer is applied to the medication bottle.

16. The system of claim 15, wherein the bottle feeding system (300) comprises:
a. a belt feeder (310) comprising a belt (320), configured to rotate around an elongated plate or elongated axis formed by a drive gear (362) operatively connected to a drive motor (360) and a belt wheel (3242);
b. a plurality of cleats (330) disposed on the belt (320) spaced a distance d2 apart;
wherein a lower end of the belt feeder (310) is configured to engage bottles housed in a bottle storage hopper such that as the belt (320) rotates the cleats (330) rise and capture one or more bottles thereon as the cleats (330) rise towards a top end of the belt feeder (310), wherein the belt feeder (310) is oriented at an angle a1 with respect to horizontal;
c. a bottle delivery guide (380) configured to deliver bottles (305) from the belt feeder (310) to the slot occupying the loading position of the dial table system (400);
d. an excess bottle return chute (312) disposed adjacent to the belt feeder (310), configured to deliver bottles to the bottle storage hopper;
e. a first guide plate (314) disposed at or near a bottom end of the belt feeder (310), configured to cause movement of bottles in an improper configuration on the belt (320) to the excess bottle return chute (312); and
f. a second guide plate (316) disposed at or near a top end of the belt feeder (310), configured to cause movement of the bottles (305) to the bottle delivery guide (380).

17. The system of claim 16, wherein the plurality of cleats (330) comprise a first set of cleats (332) and a second set of cleats (334);
wherein each cleat of the first set of cleats (332) comprises a width w3 extending from the belt (320), wherein the first set of cleats (332) are suitable for supporting the bottles (205);
wherein each cleat of the second set of cleats (334) comprises a width w4 extending from the belt (320), wherein the second set of cleats (334) are unsuitable for supporting the bottles (205);
wherein the second set of cleats (334) are alternately disposed between the first set of cleats (332).

18. The system of claim 15, wherein the clamping system (1200) comprises:
a. a base mount (1210) having a front end and a back end;
b. a clamping arm (1220) having a front end and a back end, slidably disposed on the base mount (1210);
c. an insert (1230) disposed at the front end of the clamping arm (1220), the insert (1230) comprising a concave curved shape matching a curvature of the medication bottle;
d. a motor mount (1240) operatively coupled to the back end of the base mount (1210); and
e. a clamping motor mechanism (1250) comprising a motor (1252) operatively coupled to the motor mount (1240), and a shaft (1254) operatively coupled to the motor (1252) and the back end of the clamping arm (1220), wherein the clamping motor mechanism (1250) is configured to extend the shaft (1254) such that the clamping arm (1220) applies pressure to the medication bottle, and retract the shaft (1254) such that the clamping arm (1220) does not contact the medication bottle.

19. The system of claim 15, wherein the heat sealing system (1300) comprises:
a. a frequency generator (1310), configured to generate a frequency;
b. a power supply (1320) operatively coupled to the frequency generator (1310), configured to supply power to the frequency generator (1310); and
c. a coil (1330) operatively coupled to the frequency generator (1310), configured to accept the frequency and generate a heat field such that the meltable seal melts such that the foil layer is applied to the medication bottle.

20. A medication pump accessory system for automated assembling of a medication order, said accessory system comprising:
a. a control center (190) for receiving an order for an array of doses of the medication directly or indirectly from an electronic medical record (EMR) system;
b. a dial table system (400) comprising at least four slots therein for temporarily holding a medication bottle, wherein each of the at least four slots occupies either a loading position, a dispensing position, a capping position, or an unloading position, the dial table system (400) rotates to move slots from one position to another;
c. a dispensing system (500) for aliquoting a unit of medication from a reservoir through a dispenser into a medication bottle in the slot occupying the dispensing position of the dial table system (400), the dispensing system (500) comprises a means of connecting a medication pump;
d. a capping system (600) for securely attaching a cap to a medication bottle in the capping position of the dial table system (400);
e. a labeling system for obtaining a bottle from the unloading position of the dial table system (400) and labeling said bottle;
f. a bottle feeding system (300) configured to obtain a medication bottle from a bottle storage hopper and directing the medication bottle to a slot occupying the loading position of the dial table system (400), the bottle feeding system (300) comprising:
i. a belt feeder (310) comprising a belt (320), configured to rotate around an elongated plate or elongated axis formed by a drive gear (362) operatively connected to a drive motor (360) and a belt wheel (3242);
ii. a plurality of cleats (330) disposed on the belt (320) spaced a distance d2 apart;
wherein a lower end of the belt feeder (310) is configured to engage bottles housed in a bottle storage hopper such that as the belt (320) rotates the cleats (330) rise and capture one or more bottles thereon as the cleats (330) rise towards a top end of the belt feeder (310), wherein the belt feeder (310) is oriented at an angle a1 with respect to horizontal;
iii. a bottle delivery guide (380) configured to deliver bottles (305) from the belt feeder (310) to the slot occupying the loading position of the dial table system (400);
iv. an excess bottle return chute (312) disposed adjacent to the belt feeder (310), configured to deliver bottles to the bottle storage hopper;
v. a first guide plate (314) disposed at or near a bottom end of the belt feeder (310), configured to cause movement of bottles in an improper configuration on the belt (320) to the excess bottle return chute (312); and
vi. a second guide plate (316) disposed at or near the top end of the belt feeder (310), configured to cause movement of the bottles (305) to the bottle delivery guide (380);
wherein the plurality of cleats (330) comprise a first set of cleats (332) and a second set of cleats (334);
wherein each cleat of the first set of cleats (332) comprises a width w3 extending from the belt (320), wherein the first set of cleats (332) are suitable for supporting the bottles (205);
wherein each cleat of the second set of cleats (334) comprises a width w4 extending from the belt (320), wherein the second set of cleats (334) are unsuitable for supporting the bottles (205);
wherein the second set of cleats (334) are alternately disposed between the first set of cleats (332);
g. a clamping system (1200) configured to hold the medication bottle in place in a slot of the dial table system (400), the clamping system (1200) comprising:
i. a base mount (1210) having a front end and a back end;
ii. a clamping arm (1220) having a front end and a back end, slidably disposed on the base mount (1210);
iii. an insert (1230) disposed at the front end of the clamping arm (1220), the insert (1230) comprising a concave curved shape matching a curvature of the medication bottle;
iv. a motor mount (1240) operatively coupled to the back end of the base mount (1210); and
v. a clamping motor mechanism (1250) comprising a motor (1252) operatively coupled to the motor mount (1240), and a shaft (1254) operatively coupled to the motor (1252) and the back end of the clamping arm (1220), wherein the clamping motor mechanism (1250) is configured to extend the shaft (1254) such that the clamping arm (1220) applies pressure to the medication bottle, and retract the shaft (1254) such that the clamping arm (1220) does not contact the medication bottle;
wherein the cap comprises a foil layer coupled to an interior of the cap by a meltable seal; and
h. a heat sealing system (1300) operatively coupled to the unloading position of the dial table system (400), configured to melt the meltable seal of the cap such that the foil layer is applied to the medication bottle, the heat sealing system (1300) comprising:
i. a frequency generator (1310), configured to generate a frequency;
ii. a power supply (1320) operatively coupled to the frequency generator (1310), configured to supply power to the frequency generator (1310); and
iii. a coil (1330) operatively coupled to the frequency generator (1310), configured to accept the frequency and generate a heat field such that the meltable seal melts such that the foil layer is applied to the medication bottle.
